SEQUENCE LISTING (21 sequences)

SEQ ID NO: 1 - protein, 514 aa, Artificial Sequence
  Description of Artificial Sequence Synthetic polypeptide
  amino acid sequence
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLQGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINGSLAFIRKSDEKLHNVEDKIEEILSKIYHIENEIARIKKLIGEA

SEQ ID NO: 2 - protein, 491 aa, Artificial Sequence
  Description of Artificial Sequence Synthetic polypeptide
  amino acid sequence
SSQNITEEFYQSTCSAVSKGYLSALRTGWYTSVITIELSNIKENKCNGTDAKVKLIKQELDKYKSAVTELQLLMQSTPATNNKFLGFLQGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINGSLAFIRKSDEKLHNVEDKIEEILSKIYHIENEIARIKKLIGEA

SEQ ID NO: 3 - protein, 544 aa, Artificial Sequence
  Description of Artificial Sequence Synthetic polypeptide
  amino acid sequence
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 4 - protein, 514 aa, Artificial Sequence
  Description of Artificial Sequence Synthetic polypeptide
  amino acid sequence
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLQGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINGTLAFIRKSDEKLHNVEDKIEEILSKIYHIENEIARIKKLIGEA

SEQ ID NO: 5 - protein, 514 aa, Artificial Sequence
  Description of Artificial Sequence Synthetic polypeptide
  amino acid sequence
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEKLHNVEDKIEEILSKIYHIENEIARIKKLIGEA

SEQ ID NO: 6 - protein, 28 aa, Artificial Sequence
  Description of Artificial Sequence Synthetic peptide
  amino acid sequence
EDKIEEILSKIYHIENEIARIKKLIGEA

SEQ ID NO: 7 - protein, 408 aa, Artificial Sequence
  Description of Artificial Sequence Synthetic polypeptide
  amino acid sequence
FLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 8 - protein, 84 aa, Artificial Sequence
  Description of Artificial Sequence Synthetic polypeptide
  amino acid sequence
QNITEEFYQSTCSAVSKGYLSALRTGWYTSVITIELSNIKENKCNGTDAKVKLIKQELDKYKNAVTELQLLMQSTPATNNRARR

SEQ ID NO: 9 - protein, 11 aa, Artificial Sequence
  Description of Artificial Sequence Synthetic peptide
  amino acid sequence
RPKPQQFFGLM

SEQ ID NO: 10 - protein, 364 aa, Haemophilus influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 11 - protein, 348 aa, Haemophilus influenzae
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 12 - protein, 10 aa, Haemophilus influenzae
SSHSSNMANT

SEQ ID NO: 13 - protein, 160 aa, Haemophilus influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 14 - protein, 141 aa, Haemophilus influenzae
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 15 - protein, 149 aa, Haemophilus influenzae
MKLTTQQTLKKGFTLIELMIVIAIIAILATIAIPSYQNYTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 16 - protein, 110 aa, Haemophilus influenzae
TKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 17 - protein, 275 aa, Haemophilus influenzae
MKYLLPTAAAGLLLLAAQPAMAIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 18 - protein, 253 aa, Haemophilus influenzae
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 19 - protein, 630 aa, Moraxella catarrhalis
MKTMKLLPLKIAVTSAMIIGLGAASTANAQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 20 - protein, 536 aa, Moraxella catarrhalis
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAH

SEQ ID NO: 21 - protein, 960 aa, Chimp adenovirus
MATPSMMPQWSYMHISGQDASEYLSPGLVQFARATESYFSLSNKFRNPTVAPTHDVTTDRSQRLTLRFIPVDREDTAYSYKARFTLAVGDNRVLDMASTYFDIRGVLDRGPTFKPYSGTAYNSLAPKGAPNSCEWEQEETQAVEEAAEEEEEDADGQAEEEQAATKKTHVYAQAPLSGEKISKDGLQIGTDATATEQKPIYADPTFQPEPQIGESQWNEADATVAGGRVLKKSTPMKPCYGSYARPTNANGGQGVLTANAQGQLESQVEMQFFSTSENARNEANNIQPKLVLYSEDVHMETPDTHLSYKPAKSDDNSKIMLGQQSMPNRPNYIGFRDNFIGLMYYNSTGNMGVLAGQASQLNAVVDLQDRNTELSYQLLLDSMGDRTRYFSMWNQAVDSYDPDVRIIENHGTEDELPNYCFPLGGIGVTDTYQAVKTNNGNNGGQVTWTKDETFADRNEIGVGNNFAMEINLSANLWRNFLYSNVALYLPDKLKYNPSNVDISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFESICLYATFFPMAHNTASTLEAMLRNDTNDQSFNDYLSAANMLYPIPANATNVPISIPSRNWAAFRGWAFTRLKTKETPSLGSGFDPYYTYSGSIPYLDGTFYLNHTFKKVSVTFDSSVSWPGNDRLLTPNEFEIKRSVDGEGYNVAQCNMTKDWFLVQMLANYNIGYQGFYIPESYKDRMYSFFRNFQPMSRQVVDQTKYKDYQEVGIIHQHNNSGFVGYLAPTMREGQAYPANFPYPLIGKTAVDSITQKKFLCDRTLWRIPFSSNFMSMGALSDLGQNLLYANSAHALDMTFEVDPMDEPTLLYVLFEVFDVVRVHQPHRGVIETVYLRTPFSAGNATT

(19) United States
(12) Patent Application Publication
(10) Pub. No.: US 2021/0283238 A1
(43) Pub. Date: Sep. 16, 2021

(54) NOVEL PROCESSES AND VACCINES

(71) Applicant: GLAXOSMITHKLINE BIOLOGICALS SA, Rixensart (BE)

(72) Inventors: Vincent Edwin Paul LEVET, Rixensart (BE); Frédéric Stéphane MATHOT, Rixensart (BE); Bram VUYLSTEKE, Rixensart (BE)

(73) Assignee: GLAXOSMITHKLINE BIOLOGICALS SA, Rixensart (BE)

(21) Appl. No.: 17/265,872

(22) PCT Filed: Aug. 5, 2019
(86) PCT No.: PCT/EP2019/070981

§ 371 Date: Feb. 4, 2021

(30) Foreign Application Priority Data
Aug. 7, 2018 (EP) 18187622.8

(51) Int. Cl. A61K 39/12; C12N 7/00

(52) U.S. Cl. CPC A61K 39/12; C12N 2710/10043; C12N 2760/00034; C12N 7/00

(57) ABSTRACT

A method of manufacturing a biological medicament comprising at least one biological molecule or vector is provided. One or more steps of the method are performed in an aseptic enclosure which has been surfaced sterilized using hydrogen peroxide, the steps including: formulating the biological molecule or vector with one or more excipients including an antioxidant, to produce a biological medicament comprising an antioxidant; filling containers with the biological medicament; and sealing or partially sealing the containers. Methods may be used to manufacture biological medicaments, immunogenic compositions and vaccines comprising antioxidants.

### SEQUENCE LISTING

The instant application contains an electronically submitted Sequence Listing in ASCII text file format (Name: VB66599_US_SL.txt; Size: 62,803 bytes; and Date of Creation: 20 May 2021) which is hereby incorporated by reference in its entirety.

### TECHNICAL FIELD

The present invention relates to methods for manufacturing a biological medicament comprising the addition of an antioxidant to prevent or reduce oxidation and to biological medicaments containing antioxidants and to related aspects. More particularly the invention relates to methods for manufacturing a biological medicament during which hydrogen peroxide is used in surface sterilisation of manufacturing equipment.

### BACKGROUND OF THE INVENTION

Consistency and shelf life of biological medicaments can be affected by oxidation during the manufacturing process, or during long term storage, or from process steps such as freezing, drying and freeze drying, or from a combination of these things. Oxidation can result from exposure to air or light or chemicals such as hydrogen peroxide. This applies in particular to polypeptides for example vaccine antigens, but also potentially can apply to any biological molecule that may be susceptible to oxidation and furthermore to vectors such as recombinant virus vectors.

Most highly reactive oxidants, including radicals, can react with biological materials such as proteins, DNA, RNA, lipids and carbohydrates. Not all oxidation is completely random, generally the less reactive the oxidant, the more selective is the oxidation site. For example, the fact that H₂O₂ is not very reactive compared to e.g. free radicals, means that it is more selective in its oxidation targets. Proteins and peptides may be a target for oxidants in biological systems. They can be targeted for oxidation both at the protein backbone, which can result in fragmentation of the back bone, and on the amino acid side chains. Oxidation of the side chains can lead to conformational changes and dimerization or aggregation. Oxidation can thus result in protein damage and can have serious consequences for the structure and function of the proteins. The side chains of cysteine, methionine, tryptophan, histidine and tyrosine are major targets for oxidation, in that order (Ji et al 2009, see later). The ease of oxidation of sulphur centres makes cysteine and methionine residues preferred sites for oxidation within proteins.

Vaporous Hydrogen Peroxide (VHP) technology has been used for over a decade to sterilize pharmaceutical processing equipment and clean rooms. VHP is a strong oxidizing agent that is effective against many microorganisms including bacterial spores and shows significant reduction of the bacterial burden (expressed by a minimum 6-log reduction in Geobacillus stearothermophilus).

Manufacture of vaccines and other biological containing drug products, particularly biological drug products intended for injection, is carried out under aseptic conditions. In particular the final steps such as formulation, filling and freeze drying can involve the transit of containers such as vessels containing excipients and/or vials filled with vaccine formulation or other drug product, through aseptic enclosures known as isolators which separate equipment from the external environment while certain operations are performed. To prevent any undesired contamination, isolator interior surfaces are regularly sterilized by using VHP technology. Following the sterilization step, VHP is then eliminated from the isolator by applying one or more aeration cycles. During an aeration cycle clean air displaces the air in the enclosure and optionally carries it through a catalytic converter where it is converted into water and oxygen. The clean air continues to be renewed until the residual VHP concentration reaches acceptable levels.

Oxidation of methionine is one of the major degradation pathways in many protein pharmaceuticals and thus it has been extensively studied. Peroxides such as hydrogen peroxide have been widely used for studying the kinetics and mechanisms of methionine oxidation in proteins.

Yin et al 2004, Pharmaceutical Research Vol 21, No. 12, 2377-2383 describes the use of hydrogen peroxide to look at non-site-specific oxidation of therapeutic proteins granulocyte colony-stimulating factor (G-CSF) and a human parathyroid hormone (hPTH) fragment and the effects of various antioxidants.

Ji et al 2009, J Pharmaceutical Sciences, Vol 98, No 12, 4485-4500 describes screening of stabilisers to prevent oxidation, using parathyroid hormone PTH as a model protein and hydrogen peroxide as the oxidant.

Lam et al 1997, J Pharmaceutical Sciences, Vol 86, No 11, 1250-1255 describes the use of antioxidants to prevent temperature induced methionine oxidation of recombinant humanised monoclonal antibody HER2.

Cheng et al 2016, J Pharmaceutical Sciences, Vol 105, 1837-1842 looks at the impact of hydrogen peroxide, which could be present from a number of sources including VHP, on oxidation and aggregation of proteins during lyophilisation using a model protein.

Li et al 2003 US 2003/0104996 describes formulations containing erythropoietin stabilised in the absence of albumin and with antioxidants such as methionine as a stabiliser.

Osterberg et al 1999 U.S. Pat. No. 5,962,650 describes formulations of Factor VIII with an amino acid such as methionine.

Hubbard et al 2018, J Pharmaceutical Science and Technology, doi:10.5731/pdajpst.2017.008326 “Vapor Phase Hydrogen Peroxide Sanitization of an Isolator for Aseptic Filling of Monoclonal Antibody Drug Product—Hydrogen Peroxide Uptake and Impact on Protein Quality”, looks at the impact of residual VHP on quality of a monoclonal antibody drug product and provides recommendations on the process parameters that may be controlled to reduce the risk of hydrogen peroxide uptake by the drug product.

Hambly & Gross 2009, Analytical Chemistry, 81, 7235-7242, describes oxidation of the protein apomyoglobin in the solid state after freeze drying when H₂O₂ is present.

Luo & Anderson 2006 and 2008, Pharm Research 23, 2239-2253 and J Pharm Sciences 97, 3907-3925 investigated cysteine oxidation in a freeze dried product (polyvinylpyrrolidine) and observed molecular motion and oxidation.

### SUMMARY OF THE INVENTION

We have discovered that biological medicaments, in particular certain immunogenic compositions and vaccines, can suffer from oxidation which could in turn affect consistency and/or efficacy and/or shelf life. Oxidation from exposure to air or to reagents or conditions used in manufacture, for example hydrogen peroxide used to sterilise equipment, may be responsible. A lyophilisation process used to freeze dry many vaccines or other biological medicaments, may also be responsible or may exacerbate the problem, for example through cryocentration of components of the medicament.

Furthermore, it has been found that hydrogen peroxide used in the sterilization of isolator units in vaccine production could have an impact on the vaccine product. Despite extensive purging of isolators with clean air after hydrogen peroxide sterilization, trace amounts of hydrogen peroxide remain and can be found in vials transiting the isolators and can also be absorbed into the immunogenic composition or vaccine product. This residual hydrogen peroxide can potentially cause oxidation of the components of biological medicaments that it comes into contact with.

Accordingly, there is provided a method of manufacturing a biological medicament comprising at least one biological molecule or vector, which method comprises the following steps of which one or more are performed in an aseptic enclosure which has been surface sterilized using hydrogen peroxide:
(a) formulating the biological molecule or vector with one or more excipients including an antioxidant, to produce a biological medicament comprising an antioxidant;
(b) filling containers with the biological medicament; and
(c) sealing or partially sealing the containers.

Also provided are biological medicaments produced by the methods of manufacture described herein.

Also provided is an immunogenic composition or vaccine comprising at least one antigen or a vector encoding at least one antigen, formulated with one or more excipients including methionine.

Further provided is an immunogenic composition or vaccine comprising at least one antigen or a vector encoding at least one antigen, formulated with one or more excipients including an antioxidant, wherein the immunogenic composition is freeze dried.

### BRIEF DESCRIPTION OF THE DRAWINGS

FIG. 1A and FIG. 1B: RP-HPLC Chromatograms for RSV PreF under different storage conditions and with and without antioxidants. FIG. 1A was obtained for a 0 μM spike, storage at 4° C. and at 14 days at 37 degrees C. (14D37° C., this convention is used throughout), showing that these storage conditions do not cause profile modification in samples not exposed to hydrogen peroxide. FIG. 1B was obtained for a 0 μM spike, 13.4 μM spike, 26.8 μM spike, 83.8 μM spike, 167.6 μM spike and 1676 μM spike, FC lyo after storage at 7D4° C. showing profile modification, dependent on the spiked concentration of hydrogen peroxide. The vertical order (top to bottom) at the y-axis is: 1676; 167.6; 83.8; 26.8; 13.4; and 0.

FIG. 2: Evolution of H₂O₂ concentration in liquid and lyophilised RSV PreF formulations post-spiking in the presence and absence of different antioxidants. In each series, the bars represent (left to right) spiked; 4 hours post spiking; lyo (corrected to take into account a 1.25× dilution factor after rehydration of lyophilised cake) 4° C.

FIG. 3: Model protein (Substance P) oxidation ratio after spiking with H₂O_{2.}, in each series, the bars represent (left to right) 0; 27; and 168 μM spike.

FIG. 4: Oxidation ratio of RSV PreF after spiking with H₂O, in each series, the bars represent (left to right) 0 and 27 μM spike.

FIG. 5: RP-HPLC chromatogram showing effect of N-Acetyl Cysteine on RSV PreF spiked with H₂O₂, the oxidized impurities are most prominent in the “No oxidant” (grey line).

FIG. 6: RP-HPLC chromatogram showing effect of Glutathione on RSV PreF spiked with H₂O₂, “No oxidant” (grey line).

FIG. 7: RP-HPLC chromatogram showing effect of L-Cysteine on RSV PreF spiked with H₂O₂, “No oxidant” (grey line).

FIG. 8: RP-HPLC chromatogram showing effect of Ascorbic Acid on RSV PreF spiked with H₂O₂, “No oxidant” (grey line).

FIG. 9A and FIG. 9B: RP-HPLC chromatogram showing effect of L-Methionine on RSV PreF spiked with H₂O₂, “No oxidant” (grey line).

FIG. 10: Analysis of purity of RSV PreF as the ratio of the main peak integration area to the area of all peaks in the chromatograms is given in previous figures, for the various antioxidants tested. In each series (left to right): 0 and 27 μM spike.

FIG. 11: SDS-PAGE for RSV PreF containing samples analysed by RP-HPLC—reducing conditions

FIG. 12: SDS-PAGE for RSV PreF containing samples analysed by RP-HPLC—non-reducing conditions

FIG. 13: A graphical representation of the effect of methionine addition on H₂O₂ content in lyophilized composition containing RSV PreF in the case of a 5 μM spike

FIG. 14: A graphical representation of the effect of methionine addition on H₂O₂ content in lyophilized composition containing RSV PreF in the case of a 44 μM spike

FIG. 15: Chromatogram showing Purity by RP-HPLC of RSV preF used in Example 2, to give a basal level of oxidation

FIG. 16: Evolution of RSV preF purity in lyophilized composition stored at 4° C. and 7D37° C. in the presence of increasing concentrations of methionine and following H₂O₂ spiking

FIG. 17: Evolution of Met343Ox ratio in relation to the Methionine concentration upon H₂O₂ spiking of RSV PreF

FIG. 18: Mathematically projected Met343Ox ratio in relation to increasing Methionine concentration in a composition containing RSV PreF

FIG. 19: Mass spectrometry results for protein D, Met192 oxidation over time.

FIG. 20: RP-HPLC chromatogram of oxidized protein D.

FIG. 21: Antigen profiles for protein D, UspA2 and PE-PilA, obtained by SDS-PAGE in non-reducing conditions.

FIG. 22: Mass spectrometry results for protein D, Met192 oxidation over time, with or without methionine or cysteine.

FIG. 23: RP-HPLC chromatogram of oxidized protein D, with or without methionine or cysteine.

FIG. 24: Antigen profile for protein D obtained by SDS-PAGE in non-reducing conditions, following H₂O₂ spiking and with or without methionine or cysteine.

FIG. 25: Hydrophobic variants HPLC for a composition containing Protein D, PEPilA and UspA2, with and without H₂O₂ and 5 mM methionine.

FIG. 26: Hydrophobic variants HPLC for a composition containing Protein D, PEPilA and UspA2, showing protein D peak, with H₂O₂and 10 mM methionine.

FIG. 27: Hydrophobic variants RP-HPLC % peak3, for protein D in a composition containing Protein D, PEPilA and UspA2; in the left panel non H₂O₂ oxidized samples without antioxidant; in the right panel H₂O₂ oxidized samples with methionine at different concentrations.

FIG. 28: Hydrophobic variants RP-HPLC % peak3, for protein D in a composition containing Protein D, PEPilA and UspA2, H₂O₂ oxidized samples with methionine at different concentrations.

FIG. 29: From RP-HPLC, the sum of area of peaks 1, 2 and 3.

FIG. 30: Liquid chromatography coupled mass spectrometry for protein D M192 oxidation in % after 1 month at 37° C. Left panel without H₂O₂, right panel with 1300 ng of H₂O₂ per mL before freeze drying, with or without methionine.

FIG. 31: As FIG. 30, liquid chromatography coupled mass spectrometry for protein D M192 oxidation, showing without H₂O₂ or methionine on the left, and on the right samples contained methionine plus 1300 ng of H₂O₂ per mL added before freeze drying.

FIG. 32: Adenovirus infectivity by FACS analysis, vector spiked with different concentrations of H₂O_{2.}

FIG. 33: Adenovirus integrity (DNA release) by Picogreen assay, vector spiked with different concentrations of H₂O_{2.}

FIG. 34: Adenovirus infectivity by FACS analysis, vector spiked with H₂O₂ with methionine present at different concentrations.

FIG. 35: Adenovirus integrity (DNA release) by Picogreen assay, vector spiked with H₂O₂ with methionine present at different concentrations.

FIG. 36: Adenovirus Hexon Methionine Oxidation measured by LC-MS, with and without H₂O₂ and with increasing concentrations of methionine.

### DESCRIPTION OF SEQUENCE IDENTIFIERS

SEQ ID NO: 1 A conformationally constrained RSV PreF antigen polypeptide sequence representing the RSV PreF antigen as used herein in the Examples.
SEQ ID NO: 2 A part of the preF sequence of SEQ ID NO: 1 showing the numbering of the methionines.
SEQ ID NO: 3 A further RSV preF sequence.
SEQ ID NO: 4 A further RSV PreF sequence.
SEQ ID NO: 5 A further RSV PreF sequence.
SEQ ID NO: 6 An exemplary coiled-coil (isoleucine zipper) sequence that may be used as a trimerization sequence, for example as in SEQ ID NO: 1, 4 and 5.
SEQ ID NO: 7 F1 chain of mature polypeptide produced from the precursor sequence shown in SEQ ID NO: 3.
SEQ ID NO: 8 F2 chain of mature polypeptide produced from the precursor sequence shown in SEQ ID NO: 3.
SEQ ID NO: 9 Substance P (model peptide used in the Examples)
SEQ ID NO: 10 An H. influenzae protein D sequence
SEQ ID NO: 11 A variant of protein D
SEQ ID NO: 12 A protein D fragment
SEQ ID NO: 13 An H. influenzae protein E fragment
SEQ ID NO: 14 A protein E fragment
SEQ ID NO: 15 An H. influenzae pilA sequence
SEQ ID NO: 16 A pilA fragment
SEQ ID NO: 17 A PE-pilA fusion protein
SEQ ID NO: 18 A PE-pilA fusion protein minus signal peptide
SEQ ID NO: 19 A M. catarrhalis UspA2 protein
SEQ ID NO: 20 A fragment of UspA2
SEQ ID NO: 21 ChAd155 adenovirus hexon Protein II major capsid protein

### DETAILED DESCRIPTION

We have found that residual H₂O₂ diffuses into immunogenic compositions and vaccines formulated and filled in commercial formulation/filling/transfer isolators sterilized with hydrogen peroxide, in particular where isolators have been sterilised using Vaporous Hydrogen Peroxide (VHP) technology. We have discovered that these traces can be responsible for protein oxidation, in particular oxidation of methionine residues on the protein.

We have shown by mass spectrometry that RSV preF was already naturally prone to oxidation by air, that oxidation is also linked to the freeze-drying process (leading to up to a 2-fold increase in the level of Met343Ox i.e. oxidised Methionine 343, in an exemplary preF protein) and that H₂O₂ spiking which involves introducing a defined quantity of liquid hydrogen peroxide into the formulation, designed to mimic residual VHP, further increases the oxidation levels (leading to up to a 10-fold increase of Met343Ox levels in the same preF). Furthermore, we have shown that other biological medicaments are similarly prone to oxidation. Additional examples are protein D from non-typeable H. influenzae (NTHi) in a composition containing Protein D, PEPilA and UspA2, measured by Methionine 192 oxidation (where Methionine 192 corresponds to Methionine 192 in SEQ ID NO. 14), and a live adenovirus vector as measured by oxidation of methionines on the hexon protein (five methionines designated Met270, 299, 383, 468 and 512 corresponding to Methionines 270, 299, 383, 468 and 512 from ChAd155 hexon protein II major capsid protein in SEQ ID NO. 21) and by techniques to measure the integrity and infectivity of a live virus vector.

Aseptic Enclosures and Isolator Technology

Pharmaceutical manufacturing of medicinal products including biological medicaments takes place in an aseptic environment. This may take the form of an aseptic enclosure such as a clean room, or a workstation within a clean room with barriers providing separation between the enclosure and the surrounding room limiting the contact between the work station and the clean room (sometimes known as restricted access barrier systems or RABS), or an isolator. An aseptic enclosure as described herein can be any enclosure which provides a microbiologically controlled environment free or substantially free from contamination e.g. by harmful bacteria, viruses or other microorganisms. An aseptic enclosure provides a microbiologically controlled environment for aseptic processing for producing medicinal products labelled as sterile.

The term “isolator” is generally used in this context in relation to aseptic enclosures which have been developed to more reliably control the environment. An isolator may be present within a clean room. An isolator is a unit usually having a single chamber, providing a controlled environment that maintains a barrier or enclosure around one or more pieces of equipment and/or one or more processes so that an aseptic environment can be maintained for a period of time or while a process or series of processes are carried out within the isolator. Thus, an isolator provides separation of its interior from the external environment which may be for example the surrounding cleanroom and personnel. Isolators are sometimes known as closed or open systems. Closed systems remain sealed throughout operations. Open isolator systems are designed to allow for the continuous or semi-continuous transit of materials in or out of the system during operation, through one or more openings. Openings are engineered (e.g. using continuous positive pressure within the isolator) to exclude external contamination from entering the isolator chamber. Glove ports can be provided to enable operators to perform process steps inside an isolator while still maintaining a barrier with the outside and thus without any direct contact with the interior equipment and product which is under manufacture.

In one embodiment the aseptic enclosure is a clean room which is capable of providing a Grade B internal environment according to the EU guide to Good Manufacturing Practices for sterile products manufacturing.

In a further embodiment the aseptic enclosure is a workstation within a clean room, the workstation capable of providing a Grade A internal environment according to the EU guide to Good Manufacturing Practices for sterile products manufacturing.

In another embodiment the aseptic enclosure is an isolator which is capable of providing a Grade A internal environment according to the EU guide to Good Manufacturing Practices for sterile products manufacturing.

Controlled environments for aseptic operations for pharmaceutical production are mainly provided by conventional clean rooms, of Grade B, containing workstations, of Grade A complying with the PIC/S (Pharmaceutical Inspection Co-operation Scheme) and EC guide to GMP (Good Manufacturing Practices). A smaller number of controlled environments are provided by clean rooms, of Grade D or better containing isolators providing a Grade A environment.

Air locks can be used for introducing materials into an isolator. Within an air lock sterilization may be carried out to sterilize the surfaces of containers in which the materials are present, before introducing the containers into the isolator. Aseptic enclosures such as isolators may be used to perform a variety of operations during the production of biological medicaments. One such operation is filling of vials of the product where vials are filled with the medicament and stoppered, or partially stoppered in preparation for a final step such as lyophilization. Another such operation is the simple transfer to another piece of equipment, for example the transfer of partially stoppered vials to a lyophilizer where the medicament is to be freeze dried. For vaccine production, operations performed within an aseptic enclosure such as an isolator can include, for example, coupling of a vaccine antigen or antigens to an additional antigen or to a carrier to produce a conjugated vaccine, formulation of vaccine antigens with excipients, filling of containers with bulk final vaccine formulation or filling of individual vials with one or more vaccine doses, and the transportation of filled vials to a further step such as lyophilisation (freeze drying). It will be understood that the operations relevant to the description herein are not limited and can be any operation or combination of operations performed in the production of a biological medicament which is carried out in an aseptic environment that may contain residual H₂O₂ from a hydrogen peroxide sterilization process.

Aseptic enclosures need to be regularly decontaminated, for example between operations performed on different materials, to ensure aseptic conditions for the next operation to be performed in the enclosure. A commonly used decontaminant in pharmaceutical production is hydrogen peroxide and this may be used in a variety of forms.

Vaporous or Vaporised Hydrogen Peroxide (VHP)

In one embodiment the hydrogen peroxide in the process described herein is used in the form of vaporous hydrogen peroxide which is hydrogen peroxide in the form of a vapour. This is different to aerosol hydrogen peroxide which is in the form of droplets of hydrogen peroxide in water, often referred to as dry fog.

To achieve a required level of decontamination, a defined concentration and exposure time to VHP is employed. The VHP level employed for sterilization of aseptic enclosures is generally expressed in ppm v/v (parts per million) or mg/m³ as required by safety standards globally. VHP is rated as harmful to humans and many countries have therefore imposed an occupational exposure limit. The maximum amount of hydrogen peroxide to which workers can be exposed may vary according to regulations which differ from country to country, or may be expressed in different terms from country to country. For example, in Belgium there is a Permissible Exposure Limit of 1.0 ppm v/v or 1.4 mg/m³ averaged over an 8-hour work shift whereas in the UK the limit is 2.0 ppm v/v for 15 minutes

At the end of a sterilization cycle using VHP, the room or enclosure is aerated with fresh air and an air analysis is necessary before staff are permitted to enter the room or before further materials can be introduced into an isolator for another production stage. The concentration of hydrogen peroxide must be reduced to non-hazardous levels, usually less than 1 ppm v/v or lower e.g. 0.1 ppm v/v, or between 0.1 and 1.0 ppm v/v.

Hydrogen peroxide is completely soluble in water. VHP is produced by actively vapourizing an aqueous solution of H₂O₂ and water and may be produced by a generator specifically designed for the purpose. A suitable generator comprises a vapourizing plate. The H₂O₂solution used for the production of VHP may be at a concentration of typically between 20-70% or between 30-50% or more particularly between 30-35%, for example around 35% w/w. The generator produces VHP by passing aqueous hydrogen peroxide over a vapourizer, and the vapour is then circulated at a programmed concentration in air, typically from 140 ppm to 1400 ppm (a concentration of 75 ppm is considered to be “Immediately Dangerous to Life or Health” in humans), depending on the purpose for which the aseptic enclosure is being used. Within the generator, the temperature of the air/H₂O₂/H₂O mixture is sufficiently high that it is in a gaseous state. The gas is carried from the generator into the isolator enclosure to sterilize its surfaces and render it aseptic.

After the VHP has circulated in the enclosed space for a pre-defined period of time, it is removed for example by being circulated back through the generator, where it may be broken down into water and oxygen by a catalytic converter. Alternatively, the VHP can be vented to the outside. The level of VHP in the enclosure is reduced, typically by ventilation, until concentrations of VHP fall to safe levels e.g. levels that are required for safety standards in a particular country such as Belgium or the UK. Or it may be reduced to lower levels that are required for a particular purpose which may vary according to the biological medicament in production.

In one embodiment the VHP level in the enclosure, after sterilization, is lowered until it reaches less than or equal to 1 ppm v/v, or less than or equal to 0.5 ppm v/v, or less than or equal to 0.1 ppm v/v, or between 0.05 ppm v/v and 1.0 ppm v/v, or between 0.1 ppm v/v and 1.0 ppm v/v.

The target reduced VHP levels in an enclosure such as an isolator may be achieved for example by using a defined working set point provided by the equipment.

In one embodiment the isolator has a working set point between 0.1 and 1.0 ppm v/v for VHP, meaning that the isolator can be used once the VHP is at a level below or equal to a set point in the range of 0.1 to 1.0 ppm v/v VHP.

In another embodiment the isolator has a working set point of 1.0 ppm v/v VHP, meaning that the isolator can be used once the VHP is at a level of 1.0 ppm v/v VHP or below.

In one embodiment, the measurement of residual VHP levels in an enclosure is by means of visual colorimetric tubes such as Draeger Tubes.

A typical sterilization cycle using VHP may consist of the following phases:

Phase 1—Pre-conditioning: the necessary starting conditions for surface sterilization are created in the system during a preconditioning phase (the solution is set up, vaporizing plate is prepared, optionally humidity is adjusted).

Phase 2—Conditioning: the dosage of gaseous H₂O₂ required to achieve the desired decontamination effect is generated in the enclosure.

Phase 3—Sterilization: introduction of the applied dose of VHP over a defined time.

Phase 4—Aeration: attainment of the residual H₂O₂ concentration (ppm v/v) required in the enclosure.

After the sterilization (phase 3), an aeration (phase 4) is carried out to remove or eliminate the VHP from the isolator. The maximum concentration of residual VHP allowed after the aeration phase is typically 1 ppm, as measured by visual colorimetric tubes (Draeger tubes). The VHP concentration continues to decrease while heating, ventilation and air conditioning of the enclosure continues.

Aerosol Hydrogen Peroxide (aHP)

In another embodiment hydrogen peroxide is used in the form of an aerosol (also known a dry fog) which consists of droplets of hydrogen peroxide solution in water. aHP may be introduced into an enclosure by spraying H₂O₂ solution into the enclosure via a nozzle. aHP is an older technology than VHP, but it will be clear that this and other hydrogen peroxide sterilisation techniques can also be employed in the processes described herein.

Measuring Residual Hydrogen Peroxide

In order to understand the likely amount of residual H₂O₂ present in a product or pharmaceutical formulation described herein due to use of H₂O₂ during processing, a mock production process can be performed. A worst-case scenario production process can be simulated on the equipment used for the process, where the product is replaced by water or a representative placebo solution. The production process is performed using the least favourable conditions in terms of H₂O₂ uptake; i.e. at high residual H₂O₂ concentrations and for long processing times. Subsequently the quantity of H₂O₂ in the product (water or placebo) is determined, for example using the horseradish peroxidase Amplex Red assay.

The quantity of H₂O₂ found in the product by such a method can then be used as a basis for H₂O₂ spiking experiments where H₂O₂ is added at defined concentrations to the product to assess the product's sensitivity to oxidation.

Alternatively or additionally, the potential residual H₂O₂ that could be present in a pharmaceutical formulation due to hydrogen peroxide e.g. VHP or aHP employed in sterilization cycles, and from the equipment it has come into contact with, can be calculated mathematically according to a worst case scenario. Indeed, if preliminary experiments have been performed in order to mathematically quantify and describe the different contributions to the final H₂O₂ content in the pharmaceutical formulation, these mathematical algorithms can be used to estimate the H₂O₂ quantity in the product.

The residual H₂O₂from a VHP process is initially present in vapour form in the enclosure and diffuses into the pharmaceutical formulation where there is air contact with the formulation, and once absorbed it becomes a H₂O₂solution. Residual H₂O₂ can also be present in liquid form on the materials and equipment used in pharmaceutical production and from here can transfer into the formulation, either via the gaseous state as air is circulated in the enclosure, or by direct contact. For example, some materials such as silicon are known to be porous to H₂O₂.

The preliminary experiments and the resulting mathematical calculations should take into account variable factors such as container residence time in the enclosure, component materials of equipment, surface area of formulation exposed, filling volume, residual H₂O₂ quantity in the gas phase, stoppering or partial stoppering of vials.

Mathematical algorithms can be developed for these contributions to the final H₂O₂ quantity in the pharmaceutical formulation to provide a basis on which to make the calculations for a variety of formulations and processes. See for example Vuylsteke et al 2019, J. Pharmaceutical Sciences, 1-7: “The Diffusion of Hydrogen Peroxide Into the Liquid Product During Filling Operations Inside Vaporous Hydrogen Peroxide Sterilized Isolators Can Be Predicted by a Mechanistic Model”

Antioxidants

An antioxidant for use in the process or compositions described herein is a pharmaceutically acceptable reagent that can be added to the formulation, to prevent or reduce oxidation of the biological molecule or biological vector in the process or composition.

In one embodiment the antioxidant prevents or reduces oxidation of a polypeptide such as a vaccine antigen. Methionine residues on a polypeptide such as a vaccine antigen may be vulnerable to oxidation for example oxidation due to the presence of hydrogen peroxide or simply by contact with ambient air or during a process such as lyophilization. Hydrogen peroxide may have been left over from the sterilisation of equipment used in the production of the biological medicament (residual hydrogen peroxide) and adsorbed or diffused into the formulation. The formulation may come into contact with air and/or be more vulnerable to oxidation for example during a process such as lyophilization where the formulation is freeze dried to produce a solid product (lyophilised cake).

In one embodiment the antioxidant reduces oxidation of methionine groups on a polypeptide. In a particular embodiment the antioxidant reduces the oxidation of methionine groups to a level of no more than oxidation in the absence of hydrogen peroxide. In embodiments described herein, oxidation of polypeptides can be observed or measured by methods known in the art, such as those described herein in the Examples. Oxidation of proteins can be observed or measured for example by means of mass spectrometry, RP-HPLC and SDS-PAGE. In one embodiment two of these three methods are used to observe or measure the level of oxidation, for example mass spectrometry and RP-HPLC. In another embodiment all three methods are used. In further embodiments described herein, oxidation of proteins on the surface of a virus vector can be observed or measured for example by mass spectrometry.

Examples of pharmaceutically acceptable antioxidants for use in a process and compositions such as immunogenic compositions described herein, include thiol containing excipients such as N-acetyl cysteine, L-cysteine, glutathione, monothioglycerol; and thioether containing excipients such as methionine, in the form of L-methionine or D-methionine; and ascorbic acid. Amino acid antioxidants such as methionine include monomeric or dimeric or trimeric or further multimeric forms of methionine or other amino acid, or amino acids. Multimeric amino acids may contain for example up to three or four or five or six or seven or eight amino acids in total, which may be all the same for example all methionine, or all cysteine, or may be a mixture of amino acids including for example at least one methionine or cysteine, or predominantly for example methionine or cysteine or predominantly a mixture of methionine and cysteine. Short peptides of methionine or cysteine or short peptides of a mixture of methionine are included. Such amino acid antioxidants are additives for the purpose of preventing or reducing oxidation of the polypeptide.

In certain formulations methionine is particularly effective as an antioxidant. In certain formulations methionine is further effective as an antioxidant as it does not adversely affect the purity of the antigen as measured by RP-HPLC or LC-MS.

In one embodiment the antioxidant is L-methionine.

In one embodiment the antioxidant is an antioxidant that protects against oxidation of the biological molecule or vector without adversely affect the purity of the biological molecule or vector, for example it does not result in breakdown products detectable by RP-HPLC and/or LC-MS.

In one embodiment the antioxidant is an antioxidant that protects against oxidation of a live vector such as a virus vector e.g. adenovirus vector such as ChAd155 or ChAd157, as shown or measured by vector infectivity and/or integrity. In a particular embodiment the antioxidant protects against oxidation of the vector or the effects of oxidation on the integrity or infectivity of the vector, for example as observed or measured by FACS analysis to measure expression of a transgene introduced by the vector into a host cell, and/or by a DNA quantitation assay to measure DNA release from the vector e.g. Picogreen assay.

In one embodiment the antioxidant is present at a concentration of between 0.05 mM to 50 mM in the final liquid formulation, or between 0.1 and 20 mM or 0.1 and 15 mM or 0.5 and 15 mM or 0.5 and 12 mM for example around 10 mM or around 5 mM, or between 0.1 mM and 10 mM, or between 0.1 and 5 mM, or between 0.5 mM and 5 mM or around 1 mM. Final liquid formulation refers to a liquid formulation ready for use (thus containing all of the required components), or a liquid formulation ready for freeze drying followed by reconstituting with an aqueous solution prior to use (in which case additional components such as an adjuvant may be added during reconstitution). It is not excluded that final liquid formulations may be combined with one or more further formulations prior to administration.

In one embodiment the antioxidant is present at a concentration of up to 20 mM in the final liquid formulation or up to 15 mM or up to 12 mM or up to 10 mM or up to 8 mM or up to 7 mM or up to 6 mM or up to 5 mM in the final liquid formulation.

In one embodiment the antioxidant is present at a concentration of 0.1 mM or above, or 0.5 mM or above.

In one embodiment the antioxidant is a naturally occurring amino acid or a naturally occurring antioxidant. In a particular embodiment the amino acid or naturally occurring antioxidant is a naturally occurring amino acid or naturally occurring antioxidant selected from L-methionine, L-cysteine and glutathione. In another embodiment the antioxidant is L-methionine or L-cysteine.

In one embodiment the antioxidant is methionine (e.g. L-methionine). In a particular embodiment the antioxidant is methionine (e.g. L-methionine) present at a concentration between 0.05 mM to 50 mM in the final liquid formulation, or between 0.1 and 20 mM or 0.1 and 15 mM or 0.5 and 15 mM or 0.5 and 12 mM for example around 10 mM or around 5 mM, or between 0.1 mM and 10 mM or between 0.1 and 5 mM or between 0.5 mM and 5 mM or around 1 mM.

In one embodiment the methionine (e.g. L-methionine) is present at a concentration of up to 20 mM in the final liquid formulation or up to 15 mM or up to 12 mM or up to 10 mM or up to 8 mM or up to 7 mM or up to 6 mM or up to 5 mM in the final liquid formulation.

In one embodiment the methionine (e.g. L-methionine) is present at a concentration of 0.1 mM or above, or 0.5 mM or above.

The quantity of an antioxidant that is required will depend on a variety of parameters. Dose-ranging studies are performed for each biological molecule or vector to determine the efficacy of a particular antioxidant at a range of doses and thereby select the optimal dose. Relevant parameters include for example:
the amount of residual H₂O₂ which will be linked to the equipment configuration, time elapsed since sterilization and use of the equipment, H₂O₂ threshold e.g. 1 ppm or different (this will help determine the spiking level required to test the antioxidant)
the sensitivity of the particular biological molecule or vector to oxidation by H₂O₂ or air/process steps
level of basal oxidation of the biological molecule or vector
level of maximum acceptable oxidation for a particular biological molecule or vector.

Biological Medicament

The biological medicament is a pharmaceutical formulation that contains a biological component. It can be any pharmaceutical formulation, including vaccines and immunogenic compositions, which is required to be produced under sterile conditions and which has biological components that may be susceptible to oxidation during the production process. The biological components are generally, though not necessarily, the active ingredient(s) of the biological medicament.

In one embodiment, the biological medicament is intended for administration by injection. In one embodiment the process described herein is for the production of a sterile injectable formulation, for example an injectable formulation for use in humans, such as an immunogenic composition or vaccine for administration by injection.

It will be evident that the biological medicament can also be referred to as a formulation and that it can take the form of one dose or multiple doses or bulk product in a single container. The final medicament can be liquid or solid (e.g. lyophilised) and can comprise additional pharmaceutically acceptable excipients in addition to the antioxidant. The medicament may further comprise an adjuvant.

Lyophilisation

Medicaments and formulations described herein may be in liquid or in solid form.

In one embodiment the biological medicament is in a liquid form.

In another embodiment the biological medicament is in a solid form, for example it may be freeze dried, for example for reconstitution for vaccine administration. Freeze drying is a low temperature dehydration process which involves freezing the formulation to below the triple point (the lowest temperature at which the solid, liquid and gas phases of the material can coexist), lowering pressure and removing ice by sublimation in a primary drying step and removing remaining water in a second drying step. Annealing may optionally be used prior to drying to increase the size of the ice crystals by raising and lowering the temperature. Annealing is carried out by maintaining the temperature over the glass transition temperature (Tg′) of the formulation, maintaining it for a certain amount of time, before decreasing it below the Tg′. Controlled-nucleation may also be used to increase the size of the ice crystals, with the same effect on the matrix. Lyophilisation is commonly used in vaccine manufacturing.

In an embodiment lyophilisation is carried out using the following steps:
a freezing step (below the triple point)
optionally an annealing step or a controlled nucleation step
a primary drying step
a secondary drying step.

Lyophilisation increases the concentration of components of a formulation in a process known as cryoconcentration. The resulting increase in concentration of residual hydrogen peroxide described herein may cause or accentuate a deleterious effect of the hydrogen peroxide such as oxidation of biological components e.g. polypeptides in the formulation.

The concentration (amount) of components such as antioxidant in a lyophilised formulation described herein will generally be expressed or specified in relation to the liquid formulation prior to lyophilisation.

Biological Molecules and Vectors

Biological molecules include nucleic acids, proteins, polypeptides, peptides, carbohydrates, lipids and any other component or product of an organism such as antibodies, hormones, and the like. These biological molecules may be derived from, synthesised in or extracted from biological sources, or they may be chemically synthesised to represent biological products e.g. peptides. Biological molecules further include virus like particles comprising one or more polypeptides from one or more different viruses, and bacterial spores.

Biological vectors include bacterial, yeast and viral vectors such as lentiviruses, retroviruses, adenoviruses and adeno-associated viruses. Vectors can further include replicons, such as plasmids, phagemids, cosmids, baculoviruses, bacmids, bacterial artificial chromosomes (BACs), yeast artificial chromosomes (YACs). Vectors can be recombinant vectors comprising one or more expression control sequences operatively linked to one or more recombinant nucleotide sequences to be expressed in a host cell, wherein the recombinant nucleotide sequence or sequences encode an antigen or antigens.

It will be evident to the person skilled in the art that the biological molecules and vectors to which the present teachings can be applied are wide ranging. The process described herein can potentially be applied to any biological active ingredient such as a biological molecule or vector that could be susceptible to a reduced efficacy or reduced purity or reduced shelf life due to oxidation, in particular oxidation due to the presence of hydrogen peroxide.

In one embodiment the biological molecule or vector is an antigen.

In one embodiment the antigen is an RSV antigen, such as RSV prefusion F.

In one embodiment the antigen is from Varicella Zoster virus, such as gE.

In one embodiment the antigen is from H. influenzae. In a particular embodiment the antigen is protein D, including variants of protein D such as SEQ ID No. 11.

In one embodiment the antigen is an adenovirus vector. In a particular embodiment the adenovirus vector is a chimp adenovirus vector such as ChAd155 or ChAd157, for example ChAd155-RSV e.g. as described herein in the Examples.

Primarily but not exclusively, the present invention relates to immunogenic compositions and vaccines. In particular the present invention relates to medicaments for administration by injection. In one embodiment the biological molecule or vector is derived from a micro-organism that infects a human or an animal. In another embodiment the biological molecule or vector is a protein or glycoprotein antigen derived from a micro-organism that infects a human or an animal. In one embodiment the biological molecule or vector is not an antibody or derived from an antibody. In one embodiment the biological molecule or vector is not a cytokine. In one embodiment the biological molecule or vector is not a hormone. In one embodiment the biological molecule or vector is not of human origin.

Vaccines and Immunogenic Compositions

Immunogenic compositions provided herein include an immunogenic composition comprising at least one antigen formulated with one or more excipients including methionine, which composition may or may not be freeze dried.

Further provided is an immunogenic composition comprising at least one antigen formulated with one or more excipients including an antioxidant, for example methionine, wherein the immunogenic composition is freeze dried.

In an embodiment methionine (e.g. L-methionine) is present in such immunogenic compositions between 0.05 and 50 mM, or between 0.1 and 5 mM, or about 1.0 mM, in the liquid formulation.

In a particular embodiment methionine (e.g. L-methionine) is present at a concentration between 0.05 mM to 50 mM in the final liquid formulation, or between 0.1 and 20 mM or 0.1 and 15 mM or 0.5 and 15 mM or 0.5 and 12 mM for example around 10 mM or around 5 mM, or between 0.1 mM and 10 mM or between 0.1 and 5 mM or between 0.5 mM and 5 mM or around 1 mM.

In one embodiment methionine (e.g. L-methionine) is present at a concentration of up to 20 mM in the final liquid formulation or up to 15 mM or up to 12 mM or up to 10 mM or up to 8 mM or up to 7 mM or up to 6 mM or up to 5 mM in the final liquid formulation.

In one embodiment the methionine (e.g. L-methionine) is present at a concentration of 0.1 mM or above, or 0.5 mM or above.

In one embodiment the immunogenic composition comprises an RSV prefusion F protein as described herein.

In one embodiment the immunogenic composition comprises an antigen from Varicella Zoster virus, such as gE.

In one embodiment the immunogenic composition comprises an antigen from H. influenzae. In a particular embodiment the antigen is protein D, including variants of protein D such as SEQ ID No. 11.

In one embodiment the immunogenic composition comprises an adenovirus vector. In a particular embodiment the adenovirus vector is a chimp adenovirus vector such as ChAd155 or ChAd157, for example ChAd155-RSV e.g. as described herein in the Examples.

An immunogenic composition is a composition capable of inducing an immune response, for example a humoral (e.g., antibody) and/or cell-mediated (e.g., a cytotoxic T cell) response against an antigen following delivery to a mammal, suitably a human.

Vaccines include prophylactic and therapeutic vaccines. Vaccines include subunit vaccines comprising one or more antigens optionally with an adjuvant, live vaccines for example live virus vaccines, and vaccine antigens delivered by means of a vector such as a virus vector.

Embodiments herein relating to “vaccines” or “vaccine compositions” or “vaccine formulations” of the invention are also applicable to embodiments relating to “immunogenic compositions” of the invention, and vice versa.

Vaccines and immunogenic compositions may further comprise an adjuvant. An “adjuvant” as used herein refers to a composition that enhances the immune response to an immunogen. Examples of such adjuvants include but are not limited to inorganic adjuvants (e.g. inorganic metal salts such as aluminium phosphate or aluminium hydroxide), organic adjuvants (e.g. saponins, such as QS21, or squalene), oil-in-water emulsions (e.g. MF59 or AS03, both containing squalene, or similar oil-in-water emulsions containing squalene), saponins oil-based adjuvants (e.g. Freund's complete adjuvant and Freund's incomplete adjuvant), cytokines (e.g. IL-1β, IL-2, IL-7, IL-12, IL-18, GM-CFS, and INF-γ), particulate adjuvants (e.g. immuno-stimulatory complexes (ISCOMS), liposomes, or biodegradable microspheres), virosomes, bacterial adjuvants (e.g. monophosphoryl lipid A, such as 3-de-O-acylated monophosphoryl lipid A (3D-MPL), or muramyl peptides), synthetic adjuvants (e.g. non-ionic block copolymers, muramyl peptide analogues, or synthetic lipid A), synthetic polynucleotides adjuvants (e.g polyarginine or polylysine) and immunostimulatory oligonucleotides containing unmethylated CpG dinucleotides (“CpG”).

One suitable adjuvant is monophosphoryl lipid A (MPL), in particular 3-de-O-acylated monophosphoryl lipid A (3D-MPL). Chemically it is often supplied as a mixture of 3-de-O-acylated monophosphoryl lipid A with either 4, 5, or 6 acylated chains. It can be purified and prepared by the methods taught in GB 2122204B, which reference also discloses the preparation of diphosphoryl lipid A, and 3-O-deacylated variants thereof. Other purified and synthetic lipopolysaccharides have been described (U.S. Pat. No. 6,005,099 and EP 0 729 473 B1; Hilgers et al., 1986, Int.Arch.Allergy.Immunol., 79(4):392-6; Hilgers et al., 1987, Immunology, 60(1):141-6; and EP 0 549 074 B1 I).

Saponins are also suitable adjuvants (see Lacaille-Dubois, M and Wagner H, A review of the biological and pharmacological activities of saponins. Phytomedicine vol 2 pp 363-386 (1996)). For example, the saponin Quil A (derived from the bark of the South American tree Quillaja saponaria Molina), and fractions thereof, are described in U.S. Pat. No. 5,057,540 and Kensil, Crit. Rev. Ther. Drug Carrier Syst, 1996, 12:1-55; and EP 0 362 279 B1. Purified fractions of Quil A are also known as immunostimulants, such as QS21 and QS17; methods for their production are disclosed in U.S. Pat. No. 5,057,540 and EP 0 362 279 B1. Also described in these references is QS7 (a non-haemolytic fraction of Quil-A). Use of QS21 is further described in Kensil et al. (1991, J. Immunology, 146: 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711.

Another adjuvant is an immunostimulatory oligonucleotide containing unmethylated CpG dinucleotides (“CpG”) (Krieg, Nature 374:546 (1995)). CpG is an abbreviation for cytosine-guanosine dinucleotide motifs present in DNA. CpG is known as an adjuvant when administered by both systemic and mucosal routes (WO 96/02555, EP 468520, Davis et al, J.Immunol, 1998, 160:870-876; McCluskie and Davis, J.Immunol., 1998, 161:4463-6). CpG, when formulated into vaccines, may be administered in free solution together with free antigen (WO 96/02555) or covalently conjugated to an antigen (WO 98/16247), or formulated with a carrier such as aluminium hydroxide (Brazolot-Millan et al., Proc. Natl. Acad. Sci., USA, 1998, 95:15553-8).

Adjuvants such as those described above may be formulated together with carriers, such as liposomes, oil in water emulsions (such as MF59 or AS03 or oil in water emulsions containing squalene), and/or metallic salts (including aluminum salts such as aluminum hydroxide). For example, 3D-MPL may be formulated with aluminum hydroxide (EP 0 689 454) or oil in water emulsions (WO 95/17210); QS21 may be formulated with cholesterol containing liposomes (WO 96/33739), oil in water emulsions (WO 95/17210) or alum (WO 98/15287); CpG may be formulated with alum (Brazolot-Millan, supra) or with other cationic carriers.

Combinations of adjuvants may be utilized in the present invention, in particular a combination of a monophosphoryl lipid A and a saponin derivative (see, e.g., WO 94/00153; WO 95/17210; WO 96/33739; WO 98/56414; WO 99/12565; WO 99/11241), more particularly the combination of QS21 and 3D-MPL as disclosed in WO 94/00153, or a composition where the QS21 is quenched in cholesterol-containing liposomes (DQ) as disclosed in WO 96/33739. Alternatively, a combination of CpG plus a saponin such as QS21 is an adjuvant suitable for use in the present invention. A potent adjuvant formulation involving QS21, 3D-MPL & tocopherol in an oil in water emulsion is described in WO 95/17210 and is another formulation for use in the present invention. Saponin adjuvants may be formulated in a liposome and combined with an immunostimulatory oligonucleotide. Thus, suitable adjuvant systems include, for example, a combination of monophosphoryl lipid A, preferably 3D-MPL, together with an aluminium salt (e.g. as described in WO00/23105). A further exemplary adjuvant comprises QS21 and/or MPL and/or CpG. QS21 may be quenched in cholesterol-containing liposomes as disclosed in WO 96/33739.

AS01 is an Adjuvant System containing MPL (3-O-desacyl-4′-monophosphoryl lipid A), QS21 ((Quillaja saponaria Molina, fraction 21) Antigenics, New York, N.Y., USA) and liposomes. AS01B is an Adjuvant System containing MPL, QS21 and liposomes (50 μg MPL and 50 μg QS21). AS01E is an Adjuvant System containing MPL, QS21 and liposomes (25 μg MPL and 25 μg QS21). In one embodiment, the immunogenic composition or vaccine comprises AS01. In another embodiment, the immunogenic composition or vaccine comprises AS01B or AS01E. In a particular embodiment, the immunogenic composition or vaccine comprises AS01E.

Antigens

The term ‘antigen’ is well known to the skilled person. An antigen can be a protein, polysaccharide, peptide, nucleic acid, protein-polysaccharide conjugate, molecule or hapten that is capable of raising an immune response in a human or animal. Antigens may be derived from, homologous to or synthesised to mimic molecules from viruses, bacteria, parasites, protozoa or fungi. In an alternative embodiment the antigen is derived from, homologous to or synthesised to mimic molecules from a tumour cell or neoplasia. In a further embodiment the antigen is derived from, homologous to or synthesised to mimic molecules from a substance implicated in allergy, Alzheimer's disease, atherosclerosis, obesity and nicotine-dependence.

The antigen may be any antigen susceptible to oxidation, in particular where oxidation may result in reduced efficacy or purity or shelf life. In one embodiment the antigen is a biological molecule such as a polypeptide containing amino acid residues which are be liable to oxidation, for example methionine residues. In one embodiment the antigen is a protein or glycoprotein.

The antigen may be derived from a human or non-human pathogen including, e.g., viruses, bacteria, fungi, parasitic microorganisms or multicellular parasites which infect human and non-human vertebrates, or from a cancer cell or tumour cell.

RSV Antigens

In one embodiment the antigen is a human respiratory syncytial virus (RSV) polypeptide antigen. In certain embodiments, the polypeptide antigen is an F protein polypeptide antigen from RSV for example conformationally constrained F polypeptide antigens. Conformationally constrained F proteins have been described in both the prefusion (PreF) and postfusion (PostF) conformations. Such conformationally constrained F proteins typically comprise an engineered RSV F protein ectodomain. An F protein ectodomain polypeptide is a portion of the RSV F protein that includes all or a portion of the extracellular domain of the RSV F protein and lacks a functional (e.g., by deletion or substitution) transmembrane domain, which can be expressed, e.g., in soluble (not attached to a membrane) form in cell culture.

Exemplary F protein antigens conformationally constrained in the prefusion conformation have been described in the art and are disclosed in detail in e.g., U.S. Pat. No. 8,563,002 (WO2009079796); US Published patent application No. US2012/0093847 (WO2010/149745); US2011/0305727 (WO2011/008974); US2014/0141037, WO2012/158613, WO2014/160463 (contains preF known as DS-Cav1), WO2017/109629 and WO2018/109220, each of which is incorporated herein by reference for the purpose of illustrating prefusion F polypeptides (and nucleic acids), and methods of their production. Typically, the antigen is in the form of a trimer of polypeptides. Additional publications providing examples of F proteins in the prefusion conformation include: McLellan et al., Science, Vol. 340: 1113-1117; McLellan et al., Science, Vol 342: 592-598, Rigter et al., PLOS One, Vol. 8: e71072, and Krarup et. al. Nat. Commun. 6:8143 doi: 10.1038/ncomms9143 each of which can also be used in the context of the vaccine formulations disclosed herein.

For example, an F protein polypeptide stabilized in the prefusion conformation typically includes an ectodomain of an F protein (e.g., a soluble F protein polypeptide) comprising at least one modification that stabilizes the prefusion conformation of the F protein. For example, the modification can be selected from an addition of a trimerization domain (typically to the C terminal end), deletion of one or more of the furin cleavage sites (at amino acids \tilde{ }105-109 and \tilde{ }133-136), a deletion of the pep27 domain, substitution or addition of a hydrophilic amino acid in a hydrophobic domain (e.g., HRA and/or HRB). In an embodiment, the conformationally constrained PreF antigen comprises an F2 domain (e.g., amino acids 1-105) and an F1 domain (e.g., amino acids 137-516) of an RSV F protein polypeptide with no intervening furin cleavage site wherein the polypeptide further comprises a heterologous trimerization domain positioned C-terminal to the F1 domain. Optionally, the PreF antigen also comprises a modification that alters glycosylation (e.g., increases glycosylation), such as a substitution of one or more amino acids at positions corresponding to amino acids \tilde{ }500-502 of an RSV F protein. When an oligomerization sequence is present, it is preferably a trimerization sequence. Suitable oligomerization sequences are well known in the art and include, for example, the coiled coil of the yeast GCN4 leucine zipper protein, trimerizing sequence from bacteriophage T4 fibritin (“foldon”), and the trimer domain of influenza HA. Additionally or alternatively, the F polypeptide conformationally constrained in the prefusion conformation can include at least two introduced cysteine residues, which are in close proximity to one another and form a disulfide bond that stabilizes the pre-fusion RSV F polypeptide. For example, the two cysteines can be within about 10 Å of each other. For example, cysteines can be introduced at positions 165 and 296 or at positions 155 and 290. An exemplary PreF antigen is represented by SEQ ID NO: 1.

The preF described herein in the Examples and according to SEQ ID No:1 is known to have 3 out of 7 methionines (Met 317, Met 343, Met 74) that are preferentially oxidized. Numbering of the methionines is according to SEQ ID NO: 2 and the positions of the methionines including Met317, Met343 and Met74, are shown in SEQ ID NO: 2 which is a part of SEQ ID NO:1. Of these 3 methionines, the extent of oxidation is observed in the following order: Met317>Met 343>Met 74. Met343 has been selected herein in the Examples as the most straightforward one to quantify, as it is distributed on only one peptide (IMTSK peptide) after trypsin digestion. A correlation has been observed in a vaccine comprising this preF spiked with H₂O₂ between the 3 methionine oxidation ratios, showing ±3-fold and ±0.5-fold relationships between the oxidation ratios of Met343 vs. Met317 and of Met 343 vs. Met74, respectively.

SEQ ID NO: 1
MELLILKTNAITAILAAVTLCFASSQNITEEFYQSTCSAVSKGYLSALRTG
WYTSVITIELSNIKENKCNGTDAKVKLIKQELDKYKSAVTELQLLMQSTPA
TNNKFLGFLQGVGSAIASGIAVSKVLHLEGEVNKIKSALLSTNKAVVSLSN
GVSVLTSKVLDLKNYIDKQLLPIVNKQSCSISNIETVIEFQQKNNRLLEIT
REFSVNAGVTTPVSTYMLTNSELLSLINDMPITNDQKKLMSNNVQIVRQQS
YSIMSIIKEEVLAYVVQLPLYGVIDTPCWKLHTSPLCTTNTKEGSNICLTR
TDRGWYCDNAGSVSFFPLAETCKVQSNRVFCDTMNSLTLPSEVNLCNIDIF
NPKYDCKIMTSKTDVSSSVITSLGAIVSCYGKTKCTASNKNRGIIKTFSNG
CDYVSNKGVDTVSVGNTLYYVNKQEGKSLYVKGEPIINFYDPLVFPSDEFD
ASISQVNEKINGSLAFIRKSDEKLHNVEDKIEEILSKIYHIENEIARIKKL
IGEA
SEQ ID NO: 2
SSQNITEEFYQSTCSAVSKGYLSALRTGWYTSVITIELSNIKENKCNGTDA
KVKLIKQELDKYKSAVTELQLLM₇₄QSTPATNNKFLGFLQGVGSAIASGIA
VSKVLHLEGEVNKIKSALLSTNKAVVSLSNGVSVLTSKVLDLKNYIDKQLL
PIVNKQSCSISNIETVIEFQQKNNRLLEITREFSVNAGVTTPVSTYM₁₉₈LT
NSELLSLINDM₂₁₁PITNDQKKLM₂₂₁SNNVQIVRQQSYSIM₂₃₆SIIKEEVLA
YVVQLPLYGVIDTPCWKLHTSPLCTTNTKEGSNICLTRTDRGWYCDNAGSV
SFFPLAETCKVQSNRVFCDTM₃₁₇NSLTLPSEVNLCNIDIFNPKYDCKIM₃₄₃
TSKTDVSSSVITSLGAIVSCYGKTKCTASNKNRGIIKTFSNGCDYVSNKGV
DTVSVGNTLYYVNKQEGKSLYVKGEPIINFYDPLVFPSDEFDASISQVNEK
INGSLAFIRKSDEKLHNVEDKIEEILSKIYHIENEIARIKKLIGEA

A further RSV preF molecule that may be used herein has a precursor sequence of SEQ ID NO: 3 below. The F1 and F2 chains of the processed protein are as described in SEQ ID NO: 7 and 8 below.

SEQ ID NO: 3
MELLILKANAITTILTAVTFCFASGQNITEEFYQSTCSAVSKGYLSALRTG
WYTSVITIELSNIKENKCNGTDAKVKLIKQELDKYKNAVTELQLLMQSTPA
TNNRARRELPRFMNYTLNNAKKTNVTLSKKRKRRFLGFLLGVGSAIASGVA
VCKVLHLEGEVNKIKSALLSTNKAVVSLSNGVSVLTFKVLDLKNYIDKQLL
PILNKQSCSISNIETVIEFQQKNNRLLEITREFSVNAGVTTPVSTYMLTNS
ELLSLINDMPITNDQKKLMSNNVQIVRQQSYSIMCIIKEEVLAYVVQLPLY
GVIDTPCWKLHTSPLCTTNTKEGSNICLTRTDRGWYCDNAGSVSFFPQAET
CKVQSNRVFCDTMNSLTLPSEVNLCNVDIFNPKYDCKIMTSKTDVSSSVIT
SLGAIVSCYGKTKCTASNKNRGIIKTFSNGCDYVSNKGVDTVSVGNTLYYV
NKQEGKSLYVKGEPIINFYDPLVFPSDEFDASISQVNEKINQSLAFIRKSD
ELLSAIGGYIPEAPRDGQAYVRKDGEWVLLSTFL

The bold, underlined portion of SEQ ID NO: 3 is the bacteriophage T4 fibritin (“foldon”) domain added to the RSVF ectodomain to achieve trimerization.

Another RSV PreF sequence that may be used has SEQ ID NO: 4 below. This can be found in WO2010/149745 as can SEQ ID NO: 6.

SEQ ID NO: 4
MELLILKTNAITAILAAVTLCFASSQNITEEFYQSTCSAVSKGYLSALRTG
WYTSVITIELSNIKENKCNGTDAKVKLIKQELDKYKSAVTELQLLMQSTPA
TNNKFLGFLQGVGSAIASGIAVSKVLHLEGEVNKIKSALLSTNKAVVSLSN
GVSVLTSKVLDLKNYIDKQLLPIVNKQSCSISNIETVIEFQQKNNRLLEIT
REFSVNAGVTTPVSTYMLTNSELLSLINDMPITNDQKKLMSNNVQIVRQQS
YSIMSIIKEEVLAYVVQLPLYGVIDTPCWKLHTSPLCTTNTKEGSNICLTR
TDRGWYCDNAGSVSFFPLAETCKVQSNRVFCDTMNSLTLPSEVNLCNIDIF
NPKYDCKIMTSKTDVSSSVITSLGAIVSCYGKTKCTASNKNRGIIKTFSNG
CDYVSNKGVDTVSVGNTLYYVNKQEGKSLYVKGEPIINFYDPLVFPSDEFD
ASISQVNEKINGTLAFIRKSDEKLHNVEDKIEEILSKIYHIENEIARIKKL
IGEA

A further RSV PreF sequence that may be used has SEQ ID NO: 5 below.

SEQ ID NO: 5
MELLILKTNAITAILAAVTLCFASSQNITEEFYQSTCSAVSKGYLSALRTG
WYTSVITIELSNIKENKCNGTDAKVKLIKQELDKYKSAVTELQLLMQSTPA
TNNKFLGFLLGVGSAIASGIAVSKVLHLEGEVNKIKSALLSTNKAVVSLSN
GVSVLTSKVLDLKNYIDKQLLPIVNKQSCSISNIETVIEFQQKNNRLLEIT
REFSVNAGVTTPVSTYMLTNSELLSLINDMPITNDQKKLMSNNVQIVRQQS
YSIMSIIKEEVLAYVVQLPLYGVIDTPCWKLHTSPLCTTNTKEGSNICLTR
TDRGWYCDNAGSVSFFPLAETCKVQSNRVFCDTMNSLTLPSEVNLCNIDIF
NPKYDCKIMTSKTDVSSSVITSLGAIVSCYGKTKCTASNKNRGIIKTFSNG
CDYVSNKGVDTVSVGNTLYYVNKQEGKSLYVKGEPIINFYDPLVFPSDEFD
ASISQVNEKINQSLAFIRKSDEKLHNVEDKIEEILSKIYHIENEIARIKKL
IGEA

An exemplary coiled-coil (isoleucine zipper) sequence which is found in SEQ ID NO: 1, 4 and 5 is given below as SEQ ID NO: 6

SEQ ID NO: 6
EDKIEEILSKIYHIENEIARIKKLIGEA
(F1 chain of mature polypeptide produced from the
precursor sequence shown in SEQ ID NO: 3)
SEQ ID NO: 7
FLGFLLGVGSAIASGVAVCKVLHLEGEVNKIKSALLSTNKAVVSLSNGVSV
LTFKVLDLKNYIDKQLLPILNKQSCSISNIETVIEFQQKNNRLLEITREFS
VNAGVTTPVSTYMLTNSELLSLINDMPITNDQKKLMSNNVQIVRQQSYSIM
CIIKEEVLAYVVQLPLYGVIDTPCWKLHTSPLCTTNTKEGSNICLTRTDRG
WYCDNAGSVSFFPQAETCKVQSNRVFCDTMNSLTLPSEVNLCNVDIFNPKY
DCKIMTSKTDVSSSVITSLGAIVSCYGKTKCTASNKNRGIIKTFSNGCDYV
SNKGVDTVSVGNTLYYVNKQEGKSLYVKGEPIINFYDPLVFPSDEFDASIS
QVNEKINQSLAFIRKSDELLSAIGGYIPEAPRDGQAYVRKDGEWVLLSTFL
(F2 chain of mature polypeptide produced from the
precursor sequence shown in SEQ ID NO: 3)
SEQ ID NO: 8
QNITEEFYQSTCSAVSKGYLSALRTGWYTSVITIELSNIKENKCNGTDAKV
KLIKQELDKYKNAVTELQLLMQSTPATNNRARR

VZV Antigens and Antigens from Other Sources

In another embodiment, the antigen is derived from Plasmodium spp. (such as Plasmodium falciparum), Mycobacterium spp. (such as Mycobacterium tuberculosis (TB)), Varicella Zoster Virus (VZV), Human Immunodeficiency Virus (HIV), Moraxella spp. (such as Moraxella catarrhalis) or nontypeable Haemophilus influenzae (ntHi).

In one embodiment the antigen is derived from Varicella zoster virus (VZV). A VZV antigen for use in the invention may be any suitable VZV antigen or immunogenic derivative thereof, suitably a purified VZV antigen, such at the VZV glycoprotein gE (also known as gp1) or immunogenic derivative thereof.

In one embodiment, the VZV antigen is the VZV glycoprotein gE (also known as gp1) or immunogenic derivative hereof. The wild type or full length gE protein consists of 623 amino acids comprising a signal peptide, the main part of the protein, a hydrophobic anchor region (residues 546-558) and a C-terminal tail. In one aspect, a gE C-terminal truncate (also referred to truncated gE or gE truncate) is used whereby the truncation removes 4 to 20 percent of the total amino acid residues at the carboxy terminal end. In a further aspect, the truncated gE lacks the carboxy terminal anchor region (suitably approximately amino acids 547-623 of the wild type sequence).

The gE antigen, anchorless derivatives thereof (which are also immunogenic derivatives) and production thereof is described in EP0405867 and references therein [see also Vafai A., Antibody binding sites on truncated forms of varicella-zoster virus gpl(gE) glycoprotein, Vaccine 1994 12:1265-9). EP192902 also describes gE and production thereof. Truncated gE is also described by Haumont et al. Virus Research (1996) vol 40, p 199-204, herein incorporated fully by reference. An adjuvanted VZV gE composition suitable for use in accordance of the present invention is described in WO2006/094756, i.e. a carboxy terminal truncated VZV gE in combination with adjuvant comprising QS-21, 3D-MPL and liposomes further containing cholesterol. Leroux-Roels I. et al. (J. Infect. Dis. 2012, 206: 1280-1290) reported on a phase I/II clinical trial evaluating the adjuvanted VZV truncated gE subunit vaccine.

HIV Antigens

In another embodiment the antigen is from HIV. The antigen may be an HIV protein such as a HIV envelope protein. For example, the antigen may be an HIV envelope gp120 polypeptide or an immunogenic fragment thereof, or a combination of two or more different HIV envelope gp120 polypeptides antigens or immunogenic fragments for example from different clades or strains of HIV. Other suitable HIV antigens include Nef, Gag and Pol HIV proteins and immunogenic fragments thereof. A combination of HIV antigens may be present.

Haemophilus influenzae Antigens

In another embodiment the antigen is from non-typeable Haemophilus influenzae antigen(s) for example selected from: Fimbrin protein [(U.S. Pat. No. 5,766,608—Ohio State Research Foundation)] and fusions comprising peptides therefrom [e.g. LB1(f) peptide fusions; U.S. Pat. No. 5,843,464 (OSU) or WO 99/64067];

OMP26 [WO 97/01638 (Cortecs)]; P6 [EP 281673 (State University of New York)]; TbpA and/or TbpB; Hia; Hsf; Hin47; Hif; Hmw1; Hmw2; Hmw3; Hmw4; Hap; D15 (WO 94/12641); protein D (EP 594610); P2; and P5 (WO 94/26304); protein E (WO07/084053) and/or PilA (WO05/063802). The composition may comprise Moraxella catarrhalis protein antigen(s), for example selected from: OMP106 [WO 97/41731 (Antex) & WO 96/34960 (PMC)]; OMP21; LbpA &/or LbpB [WO 98/55606 (PMC)]; TbpA &/or TbpB [WO 97/13785 & WO 97/32980 (PMC)]; CopB [Helminen M E, et al. (1993) Infect. Immun. 61:2003-2010]; UspA1 and/or UspA2 [WO 93/03761 (University of Texas)]; OmpCD; HasR (PCT/EP99/03824); PilQ (PCT/EP99/03823); OMP85 (PCT/EP00/01468); lipo06 (GB 9917977.2); lipo10 (GB 9918208.1); lipo11 (GB 9918302.2); lipo18 (GB 9918038.2); P6 (PCT/EP99/03038); D15 (PCT/EP99/03822); OmpIA1 (PCT/EP99/06781); Hly3 (PCT/EP99/03257); and OmpE.

In an embodiment, a medicament or formulation comprises non-typeable H. influenzae (NTHi) protein antigen(s) and/or M. catarrhalis protein antigen(s). The composition may comprise Protein D (PD) from H. influenzae. Protein D may be as described in WO91/18926. The composition may further comprise Protein E (PE) and/or Pilin A (PilA) from H. Influenzae. Protein E and Pilin A may be as described in WO2012/139225. Protein E and Pilin A may be presented as a fusion protein; for example LVL735 as described in WO2012/139225. For example, the composition may comprise three NTHi antigens (PD, PE and PilA, with the two last ones combined as a PEPilA fusion protein). The composition may further comprise UspA2 from M. catarrhalis. UspA2 may be as described in WO2015125118, for example MC-009 ((M)(UspA2 31-564)(HH)) described in WO2015125118. For example, the composition may comprise three NTHi antigens (PD, PE and PilA, with the two last combined as a PEPilA fusion protein) and one M. catarrhalis antigen (UspA2). Such combinations of antigens may be useful in the prevention or treatment of diseases such as chronic obstructive pulmonary disease (COPD) which is a lung disease characterized by chronic obstruction of lung airflow that interferes with normal breathing and is not fully reversible, and/or prevention or treatment of an acute exacerbation of COPD (AECOPD). AECOPD is an acute event characterised by a worsening of the patient's respiratory symptoms that is beyond normal day-to-day variations. Typically an AECOPD leads to a change in medication.

In one embodiment, the antigen is NTHi Protein D or an immunogenic fragment thereof, suitably an isolated immunogenic polypeptide with at least 70%, 80%, 85%, 90%, 91%, 92%, 93%, 94%, 95%, 96%, 97%, 98%, 99%, or 100% to Protein D sequence.

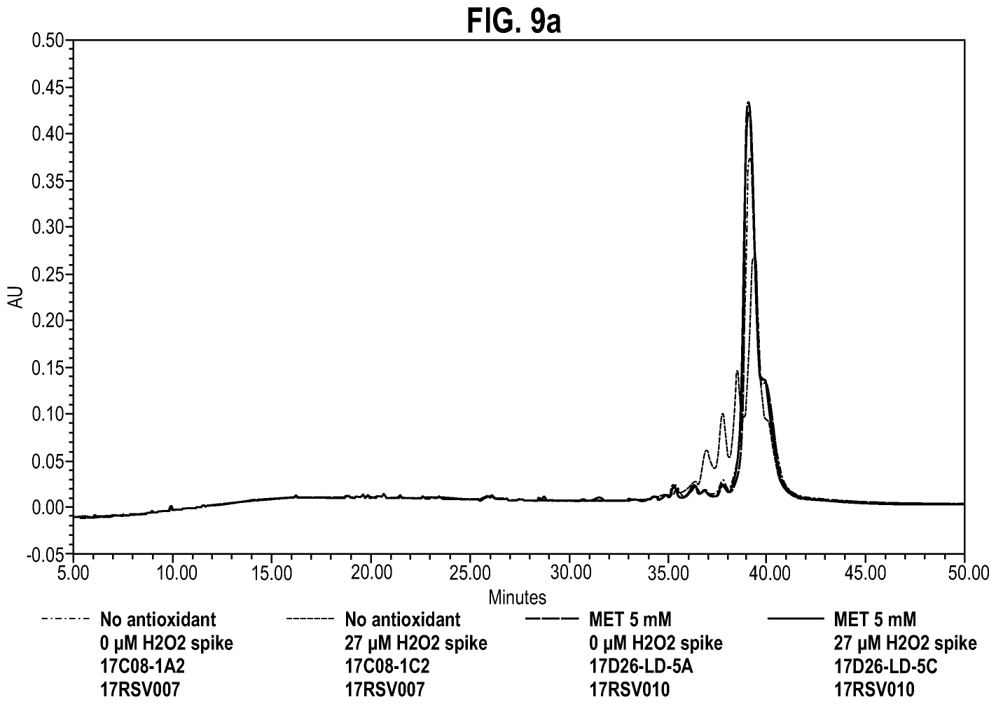

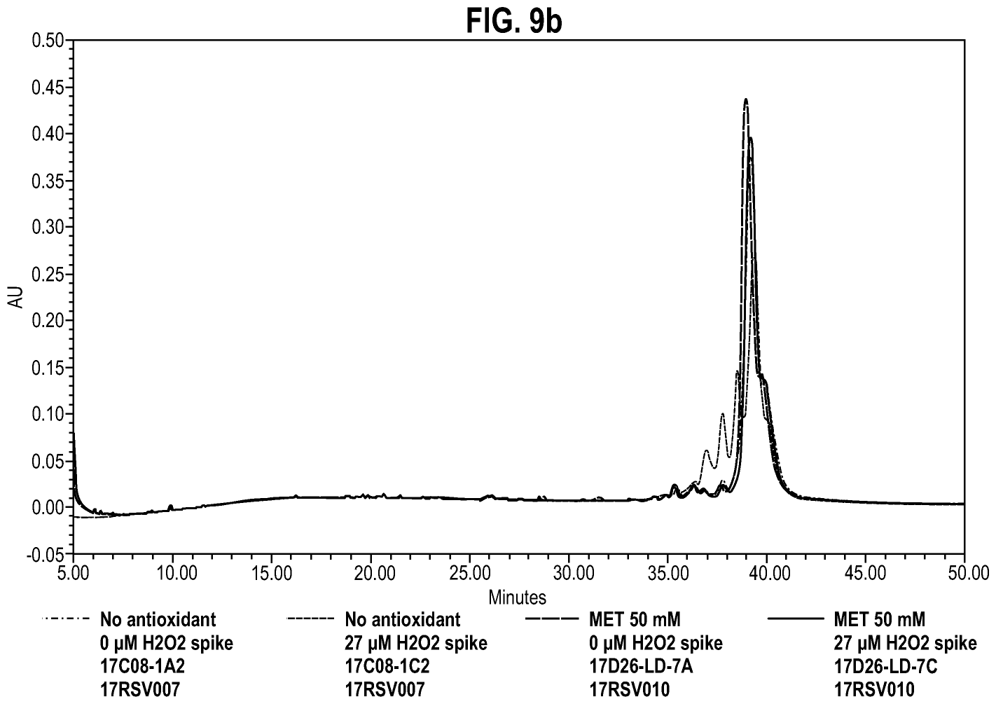

Protein D may be as described in WO91/18926. In an embodiment, the protein D has the sequence from FIG. 9 of EP 0594610 (FIGS. 9a and 9b together, 364 amino acids) (SEQ ID NO: 10 herein). This protein may provide a level of protection against Haemophilus influenzae related otitis media (Pyrmula et al Lancet 367; 740-748 (2006)). Protein D may be used as a full length protein or as a fragment (for example, Protein D may be as described in WO0056360). For example, a protein D sequence may comprise or consist of the protein D fragment described in EP0594610 which begins at the sequence SSHSSNMANT (SerSerHisSerSerAsnMetAlaAsnThr) (SEQ ID NO. 12), and lacks the 19 N-terminal amino acids from FIG. 9 of EP0594610, optionally with the tripeptide MDP from NS1 fused to the N-terminal of said protein D fragment (348 amino acids) (SEQ ID NO:11 herein). In an embodiment, the Protein D polypeptide is not conjugated to a polysaccharide, e.g. a polysaccharide from Streptococcus pneumoniae. In an embodiment, the Protein D polypeptide is a free protein (e.g. unconjugated). In one aspect, the protein D or fragment of protein D is unlipidated.

SEQ ID NO 10: Protein D (364 amino acids)
MetLysLeuLysThrLeuAlaLeuSerLeuLeuAlaAlaGlyValLeuAla
GlyCysSerSerHisSerSerAsnMetAlaAsnThrGlnMetLysSerAsp
LysIleIleIleAlaHisArgGlyAlaSerGlyTyrLeuProGluHisThr
LeuGluSerLysAlaLeuAlaPheAlaGlnGlnAlaAspTyrLeuGluGln
AspLeuAlaMetThrLysAspGlyArgLeuValValIleHisAspHisPhe
LeuAspGlyLeuThrAspValAlaLysLysPheProHisArgHisArgLys
AspGlyArgTyrTyrValIleAspPheThrLeuLysGluIleGlnSerLeu
GluMetThrGluAsnPheGluThrLysAspGlyLysGlnAlaGlnValTyr
ProAsnArgPheProLeuTrpLysSerHisPheArgIleHisThrPheGlu
AspGluIleGluPheIleGlnGlyLeuGluLysSerThrGlyLysLysVal
GlyIleTyrProGluIleLysAlaProTrpPheHisHisGlnAsnGlyLys
AspIleAlaAlaGluThrLeuLysValLeuLysLysTyrGlyTyrAspLys
LysThrAspMetValTyrLeuGlnThrPheAspPheAsnGluLeuLysArg
IleLysThrGluLeuLeuProGlnMetGlyMetAspLeuLysLeuValGln
LeuIleAlaTyrThrAspTrpLysGluThrGlnGluLysAspProLysGly
TyrTrpValAsnTyrAsnTyrAspTrpMetPheLysProGlyAlaMetAla
GluValValLysTyrAlaAspGlyValGlyProGlyTrpTyrMetLeuVal
AsnLysGluGluSerLysProAspAsnIleValTyrThrProLeuValLys
GluLeuAlaGlnTyrAsnValGluValHisProTyrThrValArgLysAsp
AlaLeuProGluPhePheThrAspValAsnGlnMetTyrAspAlaLeuLeu
AsnLysSerGlyAlaThrGlyValPheThrAspPheProAspThrGlyVal
GluPheLeuLysGlyIleLys
SEQ ID NO. 11: Protein D fragment with MDP tripep-
tide from NS1 (348 amino acids)
MetAspProSerSerHisSerSerAsnMetAlaAsnThrGlnMetLysSer
AspLysIleIleIleAlaHisArgGlyAlaSerGlyTyrLeuProGluHis
ThrLeuGluSerLysAlaLeuAlaPheAlaGlnGlnAlaAspTyrLeuGlu
GlnAspLeuAlaMetThrLysAspGlyArgLeuValValIleHisAspHis
PheLeuAspGlyLeuThrAspValAlaLysLysPheProHisArgHisArg
LysAspGlyArgTyrTyrValIleAspPheThrLeuLysGluIleGlnSer
LeuGluMetThrGluAsnPheGluThrLysAspGlyLysGlnAlaGlnVal
TyrProAsnArgPheProLeuTrpLysSerHisPheArgIleHisThrPhe
GluAspGluIleGluPheIleGlnGlyLeuGluLysSerThrGlyLysLys
ValGlyIleTyrProGluIleLysAlaProTrpPheHisHisGlnAsnGly
LysAspIleAlaAlaGluThrLeuLysValLeuLysLysTyrGlyTyrAsp
LysLysThrAspMetValTyrLeuGlnThrPheAspPheAsnGluLeuLys
ArgIleLysThrGluLeuLeuProGlnMetGlyMetAspLeuLysLeuVal
GlnLeuIleAlaTyrThrAspTrpLysGluThrGlnGluLysAspProLys
GlyTyrTrpValAsnTyrAsnTyrAspTrpMetPheLysProGlyAlaMet
AlaGluValValLysTyrAlaAspGlyValGlyProGlyTrpTyrMetLeu
ValAsnLysGluGluSerLysProAspAsnIleValTyrThrProLeuVal
LysGluLeuAlaGlnTyrAsnValGluValHisProTyrThrValArgLys
AspAlaLeuProGluPhePheThrAspValAsnGlnMetTyrAspAlaLeu
LeuAsnLysSerGlyAlaThrGlyValPheThrAspPheProAspThrGly
ValGluPheLeuLysGlyIleLys

In one embodiment, the antigen is Protein D or an immunogenic fragment thereof, suitably an isolated immunogenic polypeptide with at least 70%, 80%, 85%, 90%, 91%, 92%, 93%, 94%, 95%, 96%, 97%, 98%, 99%, or 100% to SEQ ID NO. 10. Immunogenic fragments of Protein D may comprise immunogenic fragments of at least 7, 10, 15, 20, 25, 30 or 50 contiguous amino acids of SEQ ID NO. 10. The immunogenic fragments may elicit antibodies which can bind SEQ ID NO. 10. In another embodiment, the antigen is Protein D or an immunogenic fragment thereof, suitably an isolated immunogenic polypeptide with at least 70%, 80%, 85%, 90%, 91%, 92%, 93%, 94%, 95%, 96%, 97%, 98%, 99%, or 100% to SEQ ID NO. 11. Immunogenic fragments of Protein D may comprise immunogenic fragments of at least 7, 10, 15, 20, 25, 30 or 50 contiguous amino acids of SEQ ID NO. 11.

The immunogenic composition comprising a Protein D antigen may further comprise Protein E from NTHi, or an immunogenic fragment thereof, suitably an isolated immunogenic polypeptide with at least 70%, 80%, 85%, 90%, 91%, 92%, 93%, 94%, 95%, 96%, 97%, 98%, 99%, or 100% to Protein E sequence.

Protein E (PE) is an outer membrane lipoprotein with adhesive properties. It plays a role in the adhesion/invasion of non-typeable Haemophilus influenzae (NTHi) to epithelial cells. (J. Immunology 183: 2593-2601 (2009); The Journal of Infectious Diseases 199:522-531 (2009), Microbes and Infection 10:87-96 (2008)). It is highly conserved in both encapsulated Haemophilus influenzae and non-typeable H. influenzae and has a conserved epithelial binding domain (The Journal of Infectious Diseases 201:414-419 (2010)). Thirteen different point mutations have been described in different Haemophilus species when compared with Haemophilus influenzae Rd as a reference strain. Its expression is observed on both logarithmic growing and stationary phase bacteria. (WO2007/084053).

Protein E is also involved in human complement resistance through binding vitronectin (Immunology 183: 2593-2601 (2009)). PE, by the binding domain PKRYARSVRQ YKILNCANYH LTQVR (corresponding to amino acids 84-108 of SEQ ID NO. 13), binds vitronectin which is an important inhibitor of the terminal complement pathway (J. Immunology 183:2593-2601 (2009)).

As used herein “Protein E”, “protein E”, “Prot E”, and “PE” mean Protein E from H. influenzae. Protein E may consist of or comprise the amino acid sequence of SEQ ID NO. 13 (corresponding to SEQ ID NO. 4 of WO2012/139225A1): (MKKIILTLSL GLLTACSAQI QKAEQNDVKL APPTDVRSGY IRLVKNVNYY IDSESIWVDN QEPQIVHFDA VVNLDKGLYV YPEPKRYARS VRQYKILNCA NYHLTQVRTD FYDEFWGQGL RAAPKKQKKH TLSLTPDTTL YNAAQIICAN YGEAFSVDKK) as well as sequences with at least or exactly 75%, 77%, 80%, 85%, 90%, 95%, 97%, 99% or 100% identity, over the entire length, to SEQ ID NO. 13. In one embodiment, Protein E or an immunogenic fragment thereof is suitably an isolated immunogenic polypeptide with at least 70%, 80%, 85%, 90%, 91%, 92%, 93%, 94%, 95%, 96%, 97%, 98%, 99%, or 100% to SEQ ID NO. 13. Immunogenic fragments of Protein E may comprise immunogenic fragments of at least 7, 10, 15, 20, 25, 30 or 50 contiguous amino acids of SEQ ID NO. 13. The immunogenic fragments may elicit antibodies which can bind SEQ ID NO. 13.

In another embodiment, Protein E or immunogenic fragment is suitably an isolated immunogenic polypeptide with at least 70%, 80%, 85%, 90%, 91%, 92%, 93%, 94%, 95%, 96%, 97%, 98%, 99%, or 100% to SEQ ID NO. 14 (corresponding to Seq ID No. 125 of WO2012/139225A1):

SEQ ID NO. 14: Amino acids 20-160 of Protein E
I QKAEQNDVKL APPTDVRSGY IRLVKNVNYY IDSESIWVDN
QEPQIVHFDA VVNLDKGLYV YPEPKRYARS VRQYKILNCA
NYHLTQVRTD FYDEFWGQGL RAAPKKQKKH TLSLTPDTTL
YNAAQIICAN YGEAFSVDKK

The immunogenic composition comprising a Protein D antigen may further comprise PilA, or an immunogenic fragment thereof, suitably an isolated immunogenic polypeptide with at least 70%, 80%, 85%, 90%, 91%, 92%, 93%, 94%, 95%, 96%, 97%, 98%, 99%, or 100% to PilA sequence. In another embodiment, the immunogenic composition may comprise an immunogenic fragment of PilA, suitably an isolated immunogenic polypeptide with at least 70%, 80%, 85%, 90%, 91%, 92%, 93%, 94%, 95%, 96%, 97%, 98%, 99%, or 100% to PilA sequence.

Pilin A (PilA) is likely the major pilin subunit of H. influenzae Type IV Pilus (Tfp) involved in twitching motility (Infection and Immunity, 73: 1635-1643 (2005)). NTHi PilA is a conserved adhesin expressed in vivo. It has been shown to be involved in NTHi adherence, colonization and biofilm formation. (Molecular Microbiology 65: 1288-1299 (2007)).

As used herein “PilA” means Pilin A from H. influenzae. PilA may consist of or comprise the protein sequence of SEQ ID NO. 15 (corresponding to SEQ ID NO. 58 of WO2012/139225A1) (MKLTTQQTLK KGFTLIELMI VIAIIAILAT IAIPSYQNYT KKAAVSELLQ ASAPYKADVE LCVYSTNETT NCTGGKNGIA ADITTAKGYV KSVTTSNGAI TVKGDGTLAN MEYILQATGN AATGVTWTTT CKGTDASLFP ANFCGSVTQ) as well as sequences with 80% to 100% identity to SEQ ID NO. 15. For example, PilA may be at least 80%, 85%, 90%, 95%, 97% or 100% identical to SEQ ID NO. 15. In an embodiment, the immunogenic composition may comprise PilA or an immunogenic fragment thereof, suitably an isolated immunogenic polypeptide with at least 70%, 80%, 85%, 90%, 91%, 92%, 93%, 94%, 95%, 96%, 97%, 98%, 99%, or 100% to Seq ID NO. 15.

Immunogenic fragments of PilA may comprise immunogenic fragments of at least 7, 10, 15, 20, 25, 30 or 50 contiguous amino acids of SEQ ID NO. 15. The immunogenic fragments may elicit antibodies which can bind SEQ ID NO. 15.

In another embodiment the immunogenic composition comprises an immunogenic fragment of PilA, suitably an isolated immunogenic polypeptide with at least 70%, 80%, 85%, 90%, 91%, 92%, 93%, 94%, 95%, 96%, 97%, 98%, 99%, or 100% to SEQ ID NO. 16 (corresponding to Seq ID No. 127 of WO2012/139225A1):

SEQ ID NO. 16: Amino acids 40-149 of PilA from H.
influenzae strain 86-028NP:
T KKAAVSELLQ ASAPYKADVE LCVYSTNETT NCTGGKNGIA
ADITTAKGYV KSVTTSNGAI TVKGDGTLAN MEYILQATGN
AATGVTWTTT CKGTDASLFP ANFCGSVTQ.

Protein E and Pilin A may be presented as a fusion protein (PE-PilA). In another embodiment, the immunogenic composition comprises Protein E and PilA, wherein Protein E and PilA are present as a fusion protein, suitably an isolated immunogenic polypeptide with at least 70%, 80%, 85%, 90%, 91%, 92%, 93%, 94%, 95%, 96%, 97%, 98%, 99%, or 100% to LVL-735 SEQ ID NO. 17 (corresponding to Seq ID No. 194 of WO2012/139225A1).

SEQ ID NO. 17: LVL735 (protein): (pelB sp)(ProtE aa
20-160)(GG)(PilA aa40-149):
MKYLLPTAAA GLLLLAAQPA MAIQKAEQND VKLAPPTDVR
SGYIRLVKNV NYYIDSESIW VDNQEPQIVH FDAVVNLDKG
LYVYPEPKRY ARSVRQYKIL NCANYHLTQV RTDFYDEFWG
QGLRAAPKKQ KKHTLSLTPD TTLYNAAQII CANYGEAFSV
DKKGGTKKAA VSELLQASAP YKADVELCVY STNETTNCTG
GKNGIAADIT TAKGYVKSVT TSNGAITVKG DGTLANMEYI
LQATGNAATG VTWTTTCKGT DASLFPANFC GSVTQ

In another embodiment, the immunogenic composition comprises Protein E and PilA, wherein Protein E and PilA are present as a fusion protein, suitably an isolated immunogenic polypeptide with at least 70%, 80%, 85%, 90%, 91%, 92%, 93%, 94%, 95%, 96%, 97%, 98%, 99%, or 100% to LVL-735, wherein the signal peptide has been removed, SEQ ID NO. 18 (corresponding to Seq ID No. 219 of WO2012/139225A1).

SEQ ID NO. 18: PE-PilA fusion protein without
signal peptide:
IQKAEQND VKLAPPTDVR SGYIRLVKNV NYYIDSESIW
VDNQEPQIVH FDAVVNLDKG LYVYPEPKRY ARSVRQYKIL
NCANYHLTQV RTDFYDEFWG QGLRAAPKKQ KKHTLSLTPD
TTLYNAAQII CANYGEAFSV DKKGGTKKAA VSELLQASAP
YKADVELCVY STNETTNCTG GKNGIAADIT TAKGYVKSVT
TSNGAITVKG DGTLANMEYI LQATGNAATG VTWTTTCKGT
DASLFPANFC GSVTQ

The immunogenicity of Protein E (PE) and Pilin A (PilA) polypeptides may be measured as described in WO2012/139225A1; the contents of which are incorporated herein by reference.

The immunogenic composition comprising a Protein D antigen may further comprise an immunogenic polypeptide from M. catarrhalis or an immunogenic fragment thereof. In one embodiment, the immunogenic composition comprises UspA2 or an immunogenic fragment thereof.

Ubiquitous surface protein A2 (UspA2) is a trimeric autotransporter that appears as a lollipop-shared structure in electron micrographs (Hoiczyk et al. EMBO J. 19: 5989-5999 (2000)). It is composed of a N-terminal head, followed by a stalk which ends by an amphipathic helix and a C-terminal membrane domain (Hoiczyk et al. EMBO J. 19: 5989-5999 (2000)). UspA2 contains a very well conserved domain (Aebi et al., Infection & Immunity 65(11) 4367-4377 (1997)), which is recognized by a monoclonal antibody that was shown protective upon passive transfer in a mouse Moraxella catarrhalis challenge model (Helminnen et al. J Infect Dis. 170(4): 867-72 (1994)).

UspA2 has been shown to interact with host structures and extracellular matrix proteins like fibronectin (Tan et al., J Infect Dis. 192(6): 1029-38 (2005)) and Iaminin (Tan et al., J Infect Dis. 194(4): 493-7 (2006)), suggesting it can play a role at an early stage of Moraxella catarrhalis infection.

UspA2 also seems to be involved in the ability of Moraxella catarrhalis to resist the bactericidal activity of normal human serum (Attia A S et al. Infect Immun 73(4): 2400-2410 (2005)). It (i) binds the complement inhibitor C4bp, enabling Moraxella catarrhalis to inhibit the classical complement system, (ii) prevents activation of the alternative complement pathway by absorbing C3 from serum and (iii) interferes with the terminal stages of the complement system, the Membrane Attack Complex (MAC), by binding the complement regulator protein vitronectin (de Vries et al., Microbiol Mol Biol Rev. 73(3): 389-406 (2009)).

As used herein “UspA2” means Ubiquitous surface protein A2 from Moraxella catarrhalis.

UspA2 may consist of or comprise the amino acid sequence of SEQ ID NO: 19 (from ATCC 25238) (corresponding to Seq ID No. 1 of WO2015/125118A1):

(SEQ ID NO: 19)
MKTMKLLPLKIAVTSAMIIGLGAASTANAQAKNDITLEDLPYLIKKIDQNE
LEADIGDITALEKYLALSQYGNILALEELNKALEELDEDVGWNQNDIANLE
DDVETLTKNQNALAEQGEAIKEDLQGLADFVEGQEGKILQNETSIKKNTQR
NLVNGFEIEKNKDAIAKNNESIEDLYDFGHEVAESIGEIHAHNEAQNETLK
GLITNSIENTNNITKNKADIQALENNVVEELFNLSGRLIDQKADIDNNINN
IYELAQQQDQHSSDIKTLKKNVEEGLLELSGHLIDQKTDIAQNQANIQDLA
TYNELQDQYAQKQTEAIDALNKASSENTQNIEDLAAYNELQDAYAKQQTEA
IDALNKASSENTQNIEDLAAYNELQDAYAKQQTEAIDALNKASSENTQNIA
KNQADIANNINNIYELAQQQDQHSSDIKTLAKASAANTDRIAKNKADADAS
FETLTKNQNTLIEKDKEHDKLITANKTAIDANKASADTKFAATADAITKNG
NAITKNAKSITDLGTKVDGFDSRVTALDTKVNAFDGRITALDSKVENGMAA
QAALSGLFQPYSVGKFNATAALGGYGSKSAVAIGAGYRVNPNLAFKAGAAI
NTSGNKKGSYNIGVNYEF

as well as sequences with at least or exactly 63%, 66%, 70%, 72%, 74%, 75%, 77%, 80%, 84%, 85%, 90%, 95%, 96%, 97%, 98%, 99% or 100% identity, over the entire length, to SEQ ID NO: 19.

UspA2 as described in SEQ ID NO: 19 contains a signal peptide (for example, amino acids 1 to 29 of SEQ ID NO: 19), a laminin binding domain (for example, amino acids 30 to 177 of SEQ ID NO: 19), a fibronectin binding domain (for example, amino acids 165 to 318 of SEQ ID NO: 19) (Tan et al. JID 192: 1029-38 (2005)), a C3 binding domain (for example, amino acids 30 to 539 of SEQ ID NO: 19 (WO2007/018463), or a fragment of amino acids 30 to 539 of SEQ ID NO: 19, for example, amino acids 165 to 318 of SEQ ID NO: 19 (Hallström T et al. J. Immunol. 186: 3120-3129 (2011)), an amphipathic helix (for example, amino acids 519 to 564 of SEQ ID NO: 19 or amino acids 520-559 of SEQ ID NO: 19, identified using different prediction methods) and a C terminal anchor domain (for example, amino acids 576 to 630 amino acids of SEQ ID NO: 19 (Brooks et al., Infection & Immunity, 76(11), 5330-5340 (2008)).

In an embodiment, an immunogenic fragment of UspA2 contains a laminin binding domain and a fibronectin binding domain. In an additional embodiment, an immunogenic fragment of UspA2 contains a laminin binding domain, a fibronectin binding domain and a C3 binding domain. In a further embodiment, an immunogenic fragment of UspA2 contains a laminin binding domain, a fibronectin binding domain, a C3 binding domain and an amphipathic helix.

UspA2 amino acid differences have been described for various Moraxella catarrhalis species. See for example, J Bacteriology 181(13):4026-34 (1999), Infection and Immunity 76(11):5330-40 (2008) and PLoS One 7(9):e45452 (2012). UspA2 amino acid sequences from 38 strains of Moraxella catarrhalis are given in WO2018/178264 and WO2018/178265, incorporated herein by reference.

Immunogenic fragments of UspA2 may comprise immunogenic fragments of at least 450, 490, 511, 534 or 535 contiguous amino acids of SEQ ID NO: 19. Immunogenic fragments of UspA2 may comprise or consist of for example any of the UspA2 constructs MC-001, MC-002, MC-003, MC-004, MC-005, MC-006, MC-007, MC-008, MC-009, MC-010 or MC-011 as described in WO2015/125118A1 incorporated herein by reference, e.g. MC-009 SEQ ID No. 20 herein. The immunogenic fragments may elicit antibodies which can bind the full length sequence from which the fragment is derived.

In another embodiment, the immunogenic composition may comprise an immunogenic fragment of UspA2, suitably an isolated immunogenic polypeptide with at least 70%, 80%, 85%, 90%, 91%, 92%, 93%, 94%, 95%, 96%, 97%, 98%, 99%, or 100% to a polypeptide selected from the group consisting of MC-001, MC-002, MC-003, MC-004, MC-005, MC-006, MC-007, MC-008, MC-009 (SEQ ID NO. 20), MC-010 or MC-011 e.g. MC009 SEQ ID NO. 20 (corresponding to Seq ID No. 69 of WO2015/125118A1).

MC-009 (Protein)-(M)(UspA2 31-564)(HH)
SEQ ID NO. 20
MAKNDITLEDLPYLIKKIDQNELEADIGDITALEKYLALSQYGNILALEEL
NKALEELDEDVGWNQNDIANLEDDVETLTKNQNALAEQGEAIKEDLQGLAD
FVEGQEGKILQNETSIKKNTQRNLVNGFEIEKNKDAIAKNNESIEDLYDFG
HEVAESIGEIHAHNEAQNETLKGLITNSIENTNNITKNKADIQALENNVVE
ELFNLSGRLIDQKADIDNNINNIYELAQQQDQHSSDIKTLKKNVEEGLLEL
SGHLIDQKTDIAQNQANIQDLATYNELQDQYAQKQTEAIDALNKASSENTQ
NIEDLAAYNELQDAYAKQQTEAIDALNKASSENTQNIEDLAAYNELQDAYA
KQQTEAIDALNKASSENTQNIAKNQADIANNINNIYELAQQQDQHSSDIKT
LAKASAANTDRIAKNKADADASFETLTKNQNTLIEKDKEHDKLITANKTAI
DANKASADTKFAATADAITKNGNAITKNAKSITDLGTKVDGFDSRVTALDT
KVNAFDGRITALDSKVENGMAAQAAHH

Immunogenicity of UspA2 polypeptides may be measured as described in WO2015/125118A1; the contents of which are incorporated herein by reference.

The immunogenic compositions described herein may comprise multiple antigens from NTHi and M. catarrhalis, including protein D, PE, PilA (which may be in the form of a PE-PilA fusion) and UspA2 for example:
PD 10 μg/PE-PilA (LVL735 construct, as described in WO2012/139225) 10 μg/UspA2 (MC009 construct, as described in WO2015125118) 10 μg/AS01E
PD 10 μg/PE-PilA (LVL735 construct, as described in WO2012/139225) 10 μg/UspA2 (MC009 construct, as described in WO2015125118) 3.3 μg/AS01E

The above two specific immunogenic compositions were evaluated in a mouse Moraxella catarrhalis lung inflammation model in WO2015125118 (Example 14).

Thus, in one embodiment the immunogenic composition comprises 10 μg Protein D (e.g. SEQ ID NO. 11), 10 μg PE-PilA fusion protein (e.g. SEQ ID NO. 17 or 18) and 10 μg UspA2 (e.g. SEQ ID NO. 20), with or without an adjuvant (e.g. AS01E). In another embodiment the immunogenic composition comprises 10 μg Protein D (e.g. SEQ ID NO. 11), 10 μg PE-PilA fusion protein (e.g. SEQ ID NO. 17 or 18) and 3.3 μg UspA2 (e.g. SEQ ID NO. 20), with or without an adjuvant (e.g. AS01E).

Combinations of Antigens

It will be evident that a plurality of antigens may be provided. For example, a plurality of antigens may be provided to strengthen the elicited immune response (e.g. to ensure strong protection), a plurality of antigens may be provided to broaden the immune response (e.g. to ensure protection against a range of pathogen strains or in a large proportion of a subject population) or a plurality of antigens may be provided to concurrently elicit immune responses in respect of a number of disorders (thereby simplifying administration protocols). Where a plurality of antigens is provided, these may be as distinct proteins or may be in the form of one or more fusion proteins.

Antigen Dose

Antigens may be provided in an amount of 0.1 to 200 μg per antigen per human dose, for example 0.1 to 100 μg per antigen per human dose.

A human dose may be a fixed dose for example 0.5 ml. Individual doses of vaccine may be provided in a vial, or multiple doses of vaccine, e.g. multiple 0.5 ml doses, may be provided in a single vial. Thus in one embodiment the formulation or composition described herein is provided as a single dose (e.g. 0.5 ml dose) in a vial or as multiple doses (e.g. multiples of 0.5 ml) in a single vial. The contents of the vial may be a liquid, or a solid (e.g. where the liquid formulation has been freeze dried) ready for reconstitution with an aqueous solution prior to administration.

Vectors

Suitably the term “vector” refers to a nucleic acid that has been substantially altered (e.g., a gene or functional region has been deleted and/or inactivated) relative to a wild type sequence and/or incorporates a heterologous sequence, i.e. nucleic acid obtained from a different source (also called an “insert”), and replicating and/or expressing the inserted polynucleotide sequence, when introduced into a cell (e.g., a host cell). Vectors may include any genetic element or suitable nucleic acid molecule including naked DNA, a plasmid, a virus, a cosmid, phage vector such as lambda vector, an artificial chromosome such as a BAC (bacterial artificial chromosome), or an episome. Of particular interest herein are viral vectors. Discussed in particular herein are vectors that may be useful for delivery of vaccine antigens but it will be evident that vectors are not limited and may be useful for delivery of any protein usually a heterologous protein, to cells, either for therapeutic or vaccine purposes and may alternatively be useful for delivery of antisense nucleic acids and in gene therapy.

In one embodiment the vector is a viral vector that delivers a protein, suitably a heterologous protein, to cells, either for therapeutic or vaccine purposes. Such vectors contain an expression cassette which is the combination of a selected heterologous gene (transgene) and the other regulatory elements necessary to drive translation, transcription and/or expression of the gene product in a host cell. Such viral vectors may be based on any suitable virus such as poxviruses e.g. vaccinia virus (e.g. Modified Virus Ankara (MVA)), NYVAC (derived from the Copenhagen strain of vaccinia), avipox, canarypox (ALVAC) and fowlpox (FPV), adenoviruses, adeno-associated viruses (AAV) such as AAV type 5, alphavirus (e.g., Venezuelan equine encephalitis virus (VEE), sindbis virus (SIN), semliki forest virus (SFV), and VEE-SIN chimeras), herpes virus, measles virus, vesicular stomatitis virus vectors, retroviruses e.g. lentiviruses, herpes viruses e.g. CMV, paramyxoviruses. A vector also includes expression vectors, cloning vectors and vectors that are useful to generate recombinant viruses such as adenoviruses in host cells.

Adenovirus Vectors

In one embodiment the vector is an adenovirus vector, for example an adenovirus vector encoding an antigen derived from RSV, HCV, HPV or HSV.

Adenoviruses are species-specific and occur as different serotypes, i.e. types that are not cross-neutralized by antibodies. Adenoviruses have been isolated from humans and from nonhuman simians such as chimpanzees, bonobos, rhesus macaques and gorillas. Of particular interest are simian adenovirus vectors such as chimp adenovirus vectors. Exemplary adenovirus vectors are described in WO 2010/085984, WO 2014/139587, WO 2016/198621, WO 2018/104911 and WO 2016/198599. Exemplary adenovirus vectors include ChAd155 and ChAd157.

For example, the adenovirus vector may be a chimp adenovirus vector comprising one or more deletions of or inactivated viral genes, such as E1 or other viral gene or functional region. Such a virus vector may be described as a “backbone” which may be used as is or as a starting point for additional modifications to the vector including addition of one or more sequences encoding an antigen or antigen.

The term “replication-competent” adenovirus refers to an adenovirus which can replicate in a host cell in the absence of any recombinant helper proteins comprised in the cell. Suitably, a “replication-competent” adenovirus comprises the following intact or functional essential early genes: E1A, E1B, E2A, E2B, E3 and E4. Wild type adenoviruses isolated from a particular animal will be replication competent in that animal.

The term “replication-incompetent” or “replication-defective” adenovirus refers to an adenovirus which is incapable of replication because it has been engineered to comprise at least a functional deletion (or “loss-of-function” mutation), i.e. a deletion or mutation which impairs the function of a gene without removing it entirely, e.g. introduction of artificial stop codons, deletion or mutation of active sites or interaction domains, mutation or deletion of a regulatory sequence of a gene etc, or a complete removal of a gene encoding a gene product that is essential for viral replication, such as one or more of the adenoviral genes selected from E1A, E1B, E2A, E2B, E3 and E4 (such as E3 ORF1, E3 ORF2, E3 ORF3, E3 ORF4, E3 ORF5, E3 ORF6, E3 ORF7, E3 ORF8, E3 ORF9, E4 ORF7, E4 ORF6, E4 ORF4, E4 ORF3, E4 ORF2 and/or E4 ORF1). Particularly suitably E1 and optionally E3 and/or E4 are deleted.

Adenovirus vectors (Ad) vectors include e.g., non-replicating Ad5, Adl I, Ad26, Ad35, Ad49, ChAd3, ChAd4, ChAd5, ChAd7, ChAd8, ChAd9, ChAdlO, ChAdl I, ChAdló, ChAdl7, ChAdl9, ChAd20, ChAd22, ChAd24, ChAd26, ChAd30, ChAd31, ChAd37, ChAd38, ChAd44, ChAd63, ChAd82 and ChAd155, ChAd157, ChAdOx1 and ChAdOx2 vectors or replication-competent Ad4 and Ad7 vectors.

In one embodiment the adenovirus vector is a chimp adenovirus vector such as ChAd155, encoding an RSV antigen such as an RSV F antigen and optionally one or more further RSV antigens such as an RSV N antigen and an RSV M2 antigen. In one embodiment the adenovirus vector is a ChAd155-RSV vector encoding an RSV F, an RSV N and an RSV M2 antigen.

Antigens Expressed by Vectors

Immunogens expressed by adenovirus vectors or other vectors described herein are useful to immunize a human or non-human animal against pathogens which include e.g. bacteria, fungi, parasitic microorganisms or multicellular parasites which infect human and non-human vertebrates, or against a cancer cell or tumour cell.

Immunogens expressed by vectors described herein may be any of the antigens already described.

For example, immunogens expressed by a vector may be selected from a variety of viral families. Examples of viral families against which an immune response would be desirable include Lyssaviruses such as rabies viruses, respiratory viruses such as respiratory syncytial virus (RSV) and other paramyxoviruses such as human metapneumovirus, hMPV and parainfluenza viruses (PIV).

Further examples of suitable antigens are antigens from HCV, HPV and HSV.

Rabies antigens which are useful as immunogens to immunize a human or non-human animal can be selected from the rabies viral glycoprotein (G), RNA polymerase (L), matrix protein (M), nucleoprotein (N) and phosphoprotein (P). The term “G protein” or “glycoprotein” or “G protein polypeptide” or “glycoprotein polypeptide” refers to a polypeptide or protein having all or part of an amino acid sequence of a rabies glycoprotein polypeptide. The term “L protein” or “RNA polymerase protein” or “L protein polypeptide” or “RNA polymerase protein polypeptide” refers to a polypeptide or protein having all or part of an amino acid sequence of a rabies RNA polymerase protein polypeptide. The term “M protein” or “matrix protein” or “M protein polypeptide” or “matrix protein polypeptide” refers to a polypeptide or protein having all or part of an amino acid sequence of a rabies matrix protein polypeptide. The term “N protein” or “nucleoprotein” or “N protein polypeptide” or “nucleoprotein polypeptide” refers to a polypeptide or protein having all or part of an amino acid sequence of a rabies nucleoprotein polypeptide. The term “P protein” or “phosphoprotein” or “P protein polypeptide” or “phosphoprotein polypeptide” refers to a polypeptide or protein having all or part of an amino acid sequence of a rabies phosphoprotein polypeptide.

Suitable antigens of RSV which are useful as immunogens expressed by vectors to immunize a human or non-human animal can be selected from: the fusion protein (F), the attachment protein (G), the matrix protein (M2) and the nucleoprotein (N). The term “F protein” or “fusion protein” or “F protein polypeptide” or “fusion protein polypeptide” refers to a polypeptide or protein having all or part of an amino acid sequence of an RSV Fusion protein polypeptide. Similarly, the term “G protein” or “G protein polypeptide” refers to a polypeptide or protein having all or part of an amino acid sequence of an RSV Attachment protein polypeptide. The term “M protein” or “matrix protein” or “M protein polypeptide” refers to a polypeptide or protein having all or part of an amino acid sequence of an RSV Matrix protein and may include either or both of the M2-1 (which may be written herein as M2.1) and M2-2 gene products. Likewise, the term “N protein” or “Nucleocapsid protein” or “N protein polypeptide” refers to a polypeptide or protein having all or part of an amino acid sequence of an RSV Nucleoprotein.

In one embodiment the antigens of RSV encoded in the viral vector particularly an adenovirus e.g. ChAd155, comprise an RSV F antigen and RSV M and N antigens. More specifically, the antigens are an RSV FATM antigen (fusion (F) protein deleted of the transmembrane and cytoplasmic regions), and RSV M2-1 (transcription anti-termination) and N (nucleocapsid) antigens.

In one embodiment, the immunogen may be from a retrovirus, for example a lentivirus such as the Human Immunodeficiency Virus (HIV). In such an embodiment, immunogens may be derived from HIV-1 or HIV-2.

The HIV genome encodes a number of different proteins, each of which can be immunogenic in its entirety or as a fragment when expressed by vectors of the present invention. Envelope proteins include gp120, gp41 and Env precursor gp160, for example. Non-envelope proteins of HIV include for example internal structural proteins such as the products of the gag and pol genes and other non-structural proteins such as Rev, Nef, Vif and Tat. In an embodiment the vector of the invention encodes one or more polypeptides comprising HIV Gag.

The Gag gene is translated as a precursor polyprotein that is cleaved by protease to yield products that include the matrix protein (p17), the capsid (p24), the nucleocapsid (p9), p6 and two space peptides, p2 and p1, all of which are examples of fragments of Gag.

The Gag gene gives rise to the 55-kilodalton (kD) Gag precursor protein, also called p55, which is expressed from the unspliced viral mRNA. During translation, the N terminus of p55 is myristoylated, triggering its association with the cytoplasmic aspect of cell membranes. The membrane-associated Gag polyprotein recruits two copies of the viral genomic RNA along with other viral and cellular proteins that triggers the budding of the viral particle from the surface of an infected cell. After budding, p55 is cleaved by the virally encoded protease (a product of the pol gene) during the process of viral maturation into four smaller proteins designated MA (matrix [p17]), CA (capsid [p24]), NC (nucleocapsid [p9]), and p6, all of which are examples of fragments of Gag.

Methods for Evaluating Oxidation Level of a Biological Molecule or Vector

Various methods may be used to evaluate the effects of contact with H₂O₂, and the effects of potential antioxidants, including for example the following methods:

An example of an indirect method:

The Amplex Red colourimetric method may be used to quantify H₂O₂ at different stages for example in final bulk (FB) vaccine, in final containers (FC) where containers have been filled with a vaccine dose or doses, or after reconstitution of a lyophilised product (if applicable).

Direct Methods:
Reverse Phase High Pressure Liquid Chromatography (RP-HPLC) with high resolution can be used to assess the purity of the antigen. This high resolution chromatographic method is used to separate variants of an antigen resulting from different oxidation forms. When an antigen is oxidized, hydrophilic variants can be generated and are eluted earlier on the chromatograms. A non-oxidised chromatogram would show only one peak per antigen (the pure peak), while when oxidisation has occurred, the pure peak is decreased in size and new peaks show as oxidised forms which are eluted before the non-oxidised antigen (the pure peak). This is both a qualitative measurement by observing the peaks, and a quantitative method by calculating the percentage area of the pure peak compared to the area of all the other peaks. The value obtained is therefore close to 100% for a pure product and decreases with presence of oxidation products.
Mass Spectrometry coupled to Liquid Chromatography (LC-MS) can be used to quantify the oxidation ratio of Methionine residues e.g. in an antigen. For example, for preF of SEQ ID NO: 1, out of 7 Methionine residues, 3 are readily oxidized (Met317>Met343>Met74). Met343 was shown since it is the most easily followed (distributed on a single digestion peptide) although not the most oxidizable one. In an adenovirus vector one or more methionines in the hexon protein can be used to indicate oxidation of the vector, for example in ChAd155 five of the hexon methionines were investigated for oxidation: Met270, 299, 383, 468 and 512. In a composition comprising a protein D antigen from H. influenzae (e.g. SEQ ID NO: 11) M192 was used as a probe for oxidation, since correlations can be made between M192 oxidation and the level of oxidation of the other methionines of Protein D.
Other methods able to detect if oxidation affects the product's potential critical quality attributes (pCQA)
   Antigenicity (ELISA, Surface-Plasmon Resonance (SPR), Gyros)
   Conformation (Fourier-Transform Infrared Resonance (FTIR), Circular Dichroism (CD))

Further methods for use with live vectors to look at the impact of H₂O₂ and antioxidants include:
A DNA release assay such as the Picogreen assay can be used to measure DNA release and is thus an indication of virus capsid integrity.
Virus infectivity can be measured by looking at transgene expression in an infected host cell e.g. using FACS analysis.

Embodiments of the invention are further described in the subsequent numbered paragraphs:
1. A method of manufacturing a biological medicament comprising at least one biological molecule or vector, which method comprises the following steps of which one or more are performed in an aseptic enclosure which has been surfaced sterilized using hydrogen peroxide:
   (a) formulating the biological molecule or vector with one or more excipients including an antioxidant, to produce a biological medicament comprising an antioxidant;
   (b) filling containers with the biological medicament; and
   (c) sealing or partially sealing the containers.
2. The method according to paragraph 1, wherein the hydrogen peroxide used for sterilization is in vaporous form (VHP) or aerosolized form (aHP).
3. The method according to paragraph 1 or paragraph 2, wherein the biological molecule or vector comprises a polypeptide.
4. The method according to paragraph 1 to 3 wherein the biological molecule is a recombinant protein.
5. The method according to paragraphs 1 to 4 wherein the biological molecule or vector is susceptible to oxidation.
6. The method according to paragraphs 3 to 5 wherein the biological molecule or vector comprises one or more methionine groups and wherein the antioxidant reduces oxidation of one or more methionine groups on the biological molecule caused by the hydrogen peroxide.
7. The method according to paragraph 6 wherein the antioxidant reduces the oxidation of methionine groups to a level of no more than oxidation in the absence of hydrogen peroxide.
8. The method according to paragraphs 1 to 7 wherein the antioxidant is an amino acid.
9. The method according to paragraphs 1 to 8 wherein the antioxidant is a thioether containing molecule.
10. The method according to paragraph 9 wherein the antioxidant is methionine.
11. The method according to paragraph 10 wherein the antioxidant is L-methionine.
12. The method according to paragraphs 1 to 11 wherein the antioxidant is present in the formulation above 0.05 mM.
13. The method according to paragraphs 1 to 12 wherein the antioxidant is present in the formulation below 50 mM.
14. The method according to paragraphs 1 to 13 wherein the aseptic enclosure is an isolator.
15. The method according to paragraph 14 wherein the isolator has working set points between 0.1 and 1.0 ppm for VHP.
16. The method according to paragraph 15 wherein the isolator has a working set point at 1.0 ppm VHP.
17. The method according to paragraphs 1 to 16 wherein the biological medicament is an immunogenic composition or vaccine and the biological molecule or vector is an antigen or a vector encoding an antigen.
18. The method according to paragraph 17 wherein the antigen is an RSV antigen.
19. The method according to paragraph 18 wherein the antigen is an RSV prefusion F antigen.
20. The method according to paragraph 17 wherein the antigen is from Varicella Zoster virus.
21. The method according to paragraph 20 wherein the antigen is a VZV gE antigen.
22. The method according to paragraph 17 wherein the antigen is from H. influenzae.
23. The method according to paragraph 22 wherein the antigen is an H. influenzae protein D antigen (e.g. SEQ ID NO. 11).
24. The method according to paragraph 17 wherein the vector encoding an antigen is an adenovirus vector such as ChAd155.
25. The method according to paragraph 24 wherein the adenovirus vector encodes an RSV antigen.
26. The method according to paragraph 24 wherein the adenovirus vector encodes an antigen from Moraxella catarrhalis.
27. The method according to paragraphs 1 to 26 comprising the further step of lyophilising (freeze drying) the formulation.
28. The method according to paragraph 27 wherein the lyophilising includes the following steps:
   a freezing step (below the triple point)
   optionally an annealing step and/or a controlled nucleation step
   a primary drying step
   a secondary drying step.
29. The method according to paragraphs 1 to 28 wherein the biological medicament is a sterile injectable formulation (when in liquid form).
30. A biological medicament produced by the method according to paragraphs 1 to 29.
31. An immunogenic composition or vaccine comprising at least one antigen or a vector encoding at least one antigen, formulated with one or more excipients including methionine.
32. The immunogenic composition or vaccine of paragraph 31 comprising an RSV prefusion F antigen.
33. The immunogenic composition or vaccine of paragraph 31 comprising an H. influenzae protein D antigen (e.g. SEQ ID NO. 11).
34. The immunogenic composition or vaccine of paragraph 33 further comprising a PE-PilA fusion protein (e.g. SEQ ID NO. 17 or 18) and a M. catarrhalis UspA2 antigen (e.g. SEQ ID NO. 20).
35. The immunogenic composition or vaccine of paragraph 31 comprising an adenovirus vector such as ChAd155.
36. The immunogenic composition or vaccine of paragraphs 31 to 35 wherein methionine is present between 0.05 and 50 mM.
37. The immunogenic composition or vaccine of paragraph 36 wherein methionine is present between 0.1 and 20 mM.
38. The immunogenic composition or vaccine of paragraph 37 wherein the methionine is present between 0.1 and 15 mM.
39. The immunogenic composition or vaccine of paragraph 38 wherein the methionine is present between 0.5 and 15 mM.
40. The immunogenic composition or vaccine of paragraph 38 wherein the methionine is present between 0.1 and 5 mM.
41. The immunogenic composition or vaccine of paragraphs 31 to 40 wherein the composition is in freeze dried form.
42. The immunogenic composition or vaccine of paragraph 41, suitable for reconstitution in an aqueous solution e.g. an aqueous solution comprising an adjuvant.
43. An immunogenic composition or vaccine comprising at least one antigen or a vector encoding at least one antigen, formulated with one or more excipients including an antioxidant, wherein the immunogenic composition is freeze dried.
44. The immunogenic composition or vaccine of paragraph 43 wherein the antioxidant is a naturally occurring antioxidant.
45. The immunogenic composition or vaccine of paragraph 44 wherein the antioxidant is an amino acid.
46. The immunogenic composition or vaccine of paragraph 45 wherein the antioxidant is methionine.
47. The immunogenic composition or vaccine of paragraph 46 wherein the methionine is present between 0.05 and 50 mM in the liquid formulation before freeze drying.
48. The immunogenic composition or vaccine of paragraph 47 wherein the methionine is present between 0.1 and 20 mM in the liquid formulation before freeze drying.
49. The immunogenic composition or vaccine of paragraph 48 wherein the methionine is present between 0.1 and 15 mM before freeze drying.
50. The immunogenic composition or vaccine of paragraph 49 wherein the methionine is present between 0.5 and 15 mM before freeze drying.
51. The immunogenic composition or vaccine of paragraph 49 wherein the methionine is present between 0.1 and 5 mM before freeze drying.
52. The immunogenic composition or vaccine of paragraphs 43 to 51 suitable for reconstitution with an aqueous solution such as an aqueous solution comprising an adjuvant.
53. The immunogenic composition or vaccine of paragraph 52, reconstituted with an aqueous solution such as an aqueous solution comprising an adjuvant.
54. The immunogenic composition or vaccine of paragraphs 43 to 53 comprising an RSV prefusion F antigen.
55. The immunogenic composition or vaccine of paragraphs 43 to 53 comprising an H. influenzae protein D antigen (e.g. SEQ ID NO. 11).
56. The immunogenic composition or vaccine of paragraph 55, further comprising a PE-PilA fusion protein (e.g. SEQ ID NO. 17 or 18) and a M. catarrhalis UspA2 antigen (e.g. SEQ ID NO. 20).
57. The immunogenic composition or vaccine of paragraph 56, reconstituted with an adjuvant e.g. ASO1E.
58. The immunogenic composition or vaccine of paragraphs 43 to 53 comprising an adenovirus vector such as ChAd155.

The disclosure will be further elaborated by reference to the following Examples.

### EXAMPLES

Glossary of Terms Used in the Examples:

| | |
|---|---|
| AOx | Antioxidant |
| CYS | L-Cysteine |
| DP | Drug Product |
| DS | Drug Substance |
| EDTA | Edetate sodium/disodium |
| EIC | Extracted Ion Chromatography |
| FB | Final Bulk: unfilled final formulation, before filling |
| FC liq | Final Container liquid: vial containing the filled final |
| | bulk |
| FC lyo | Final Container lyophilized product: vial containing |
| | the lyophilized cake after freeze-drying |
| GSH | Glutathione |
| His | L-Histidine |
| HP | Hydrogen Peroxide |
| [H₂O₂] | Hydrogen Peroxide concentration |
| HRP | Horseradish Peroxidase reaction used to quantify |
| | H₂O₂ |
| MET | L-Methionine (this is what is used in these Examples) |
| Met343Ox | LC-MS method quantifying the oxidized Met343 vs. |
| | the total Met343 ratio on the preF protein. Non- |
| | qualified analytical method. |
| MSG | Glutamate monosodium |
| NAC | N-Acetyl Cysteine |
| RP-HPLC | Reverse-phase high-pressure liquid |
| | chromatography, used to assess the Purity of RSV |
| | preF2 in drug product. |
| RV | Reconstituted Vaccine |
| SP | Substance P |
| VHP | Vaporous Hydrogen Peroxide |

### Example 1

### Assessment of the Impact of Residual HP on the RSV preF2 Antigen and Selection of an Antioxidant to Protect the Antigen from Oxidation

Introduction:

A strategy was designed to assess the impact of residual HP on vaccines, which included mimicking the HP exposure by introduction of representative amounts of liquid HP (spiking) after the formulation of the final bulk (FB) during the vaccine production process. This was then followed by a vial filling step, a vial stoppering step (full stoppering for liquid vaccines or partial stoppering for lyophilized vaccines), a lyophilization process (if necessary) and a vial capping step.

In the case of lyophilized vaccines there is an initial freezing step following the exposure to residual HP. This step cryoconcentrates both the solubilized HP and the vaccine content (i.e. antigen and other formulation components) and can be considered as a worst-case scenario which can potentiate the oxidation from HP.

To understand the phenomenon and assess the impact of HP on a formulated antigen, the full process therefore needs to be mimicked as well. To include all possible elements of the vaccine manufacturing process where residual HP may affect the vaccine, the following steps may be used:
(i) Spiking with H₂O₂—with amounts of hydrogen peroxide potentially found after the filling step i.e. in final container liquid (FC liquid), which is
   right before the full stoppering step for liquid vaccines and
   right before loading in the freeze-dryer for lyophilized vaccines

but also at higher concentrations (to study the oxidation behaviour)
(ii) Maintaining a hold-time between HP spiking and loading onto freeze-dryer shelves, representative of production procedures
(iii) Performing a standard lyophilization cycle (to expose the product to a representative cryoconcentration step)
(iv) Simulate ageing of final container lyophilised product (FC lyo) before analysis (to force the oxidation reaction)

At the same time, vaccine formulations were screened in the presence and absence of antioxidants in order to understand if the addition of antioxidants could be effective in preventing the effects of the residual HP on the RSV preF2 antigen. In this case, the addition of antioxidants was performed during the final bulk production, this being the closest point to first potential exposure of the RSV preF2 to hydrogen peroxide in commercial production facilities. The antioxidant addition could also be performed prior to this (e.g. during antigen production) if exposure to a source of oxidation such as HP is expected.

The concentrations of H₂O₂ that were used for spiking were defined based on the expected amounts of H₂O₂ to be found after a manufacturing process in an isolator operated at a residual VHP concentration of 1 ppm VHP. This representative concentration would typically vary depending on the manufacturing plant design specificities, and on the security margins applied to ensure performing a study simulating worst-case conditions.

In this case, an amount of H₂O₂ higher than what would be representative of the maximum VHP was also used to help characterize the oxidation behaviour of the antigen (i.e. 168.0 μM spike).

TABLE 1
Key VHP concentrations (ppm) and corresponding
H₂O₂ (μM) used in this Example

| | Corresponding representative |
|---|---|
| [VHP] isolator limits (ppm) | [H₂O₂] spiking (μM) |
| 1.0 | 26.8 |
| Higher concentration | 168.0 |
| non-representative of VHP isolator limit | |

Methods

Assessment of the Oxidation of the RSV preF2 Antigen

The oxidation of the RSV preF2 antigen were measured through two direct analytical methods and an indirect one:

Mass-spectrometry coupled to liquid chromatography (LC-MS), which was used to quantify the ratio of oxidized methionine 343 (Met343Ox) over the total amount of the same methionine residue on the RSV preF2 protein. This method showed a non-linear impact of [H₂O₂] on RSV preF2 oxidation (saturation phenomenon at high concentrations). RSV preF2 is known to have 3 out of 7 methionines (Met 317, Met 343, Met 74) that are preferentially oxidized in the following order: Met317>Met 343>Met 74. Met343 was been selected here as the easiest one to quantify, as it is distributed on only one peptide (IMTSK peptide) after sample digestion with trypsin. Note: A correlation was observed on the Drug Substance (DS) spiked with H₂O₂ between the 3 Methionine oxidation ratios, showing ±3-fold and ±0.5-fold relationships between the oxidation ratios of Met343 vs. Met317 and of Met 343 vs. Met74, respectively.

Reverse-phase high-pressure liquid chromatography, performed in reducing conditions assessed the purity of the antigen, thanks to its ability to separate hydrophilic variants of the protein (typically produced by oxidation). It can also provide some information on the impact of the antioxidant addition on the antigen structure.

Amplex red-Horseradish Peroxidase (HRP) assay—The fate of H₂O₂ was determined by the Amplex red-HRP assay as an indirect method to quantify the H₂O₂ present at the different process steps (i.e. in FC liquid, in FC lyo, after simulated ageing).

SDS-PAGE performed in reduced and non-reduced conditions was used to determine the impact of residual HP and of the antioxidant addition on the structure of the RSV preF2 antigen.

In a specific sub-experiment, LC-EIC-MS of substance P was also used to determine the oxidation ratio of substance P as a model protein added to RSV preF2 formulation and co-lyophilized. It was used as a screening tool to evaluate the antioxidant potency.

Initial Antioxidant Selection for Experimental Screening (and Initial Doses)

10 antioxidants and the maximum concentrations at which they could be administered was established based on literature. Experimental screening then aimed at establishing the effect on pH of the addition of these excipients in the RSV preF2 vaccine composition to further select the maximum concentration at which they could be added into the vaccine formulation.

Sample Production and Management

The general schematics of the sample production and management in the experiment was as shown below in the flow diagram:
FC liq (500 μL) were formulated directly in 3 mL siliconized vials
12 different antioxidant conditions (including 1 no antioxidant and 2 different concentrations of MET) were screened (Table 2)
3 different [H₂O₂] spiking (10 μL) were then performed in FC liq following the formulation step (0, 27, 168 μM)
A 4-hour exposure of FC liq, considered as a worst-case scenario in commercial facilities was maintained before loading of vials in the freeze-dryer. During the hold-time, samples were kept in the dark.
The freeze-dryer had its shelves pre-cooled. The cycle that was performed included a freezing step, a primary drying step and a secondary drying step and lasted 45 h in total.
   Samples were then stored in the dark at 4° C.
1 arm was dedicated to H₂O₂ quantification, first in FC liq then in FC lyo (arm #1, 1 vial per antioxidant condition, per spiking and per timepoint).
1 arm was dedicated to LC-MS for Met343 oxidation ratio determination, RP-HPLC and SDS-PAGE manipulations (arm #2, 2 vials per antioxidant condition, per spiking and per timepoint).
In a specific sub-experiment, 1 arm was formulated with substance P as a model protein in the formulation, co-lyophilised (arm #3, 1 vial per antioxidant condition, per spiking and per timepoint).
FC lyo of arm #1 were stored at 4° C. before [H₂O₂] quantification.
FC lyo of arm #2 and #3 were stored at 7D37° C. before analysis (forced aging conditions).

TABLE 2
List of tested Antioxidants and selected concentrations
in Final Bulk vaccine for Example 1.

| | CONCENTRATION SELECTED FOR |
|---|---|
| ANTIOXIDANT | FORMULATION IN FB IN BOLD (mM) |
| ASCORBIC ACID | 30 |
| CITRATE, 3Na | 30 |
| CYS | 50 |
| EDTA | 5 |
| GSH | 5 |
| HIS | 50 |
| L-CYSTINE | 2.5 |
| MET | 5 and 50 |
| MSG | 50 |
| NAC | 5 |

H₂O₂ Consumption by HRP in FC Liquid vs. FC Lyo (Arm #1)

As shown above, remaining H₂O₂ was quantified at different steps during the formulation, first at the FC liq step 4 h after H₂O₂ spiking and in FC lyo (following storage at 4° C. for 10 D), using 150 mM NaCl as the reconstitution medium. Quantification was not done after 7D37° C. storage as no H₂O₂ could be found in previous experiments under these storage conditions (data not shown).

Oxidation Ratio of Substance P as a Model Protein by LC-EIC-MS (Arm #3)

Substance P (SP) is a small neuropeptide of 11 amino-acids (undecapeptide) of the Tachykinin peptides family. The sequence of Substance P is: Arg-Pro-Lys-Pro-Gln-Gln-Phe-Phe-Gly-Leu-Met, shown herein as:

SEQ ID NO: 9 RPKPQQFFGLM

Substance P was used in this sub-experiment as a model oxidizable protein having a single MET amino-acid. The MET residue is freely accessible because of the peptide's small size and because of its location in the N-terminal region of the peptide.

A direct method able to quantify the oxidation ratio of SP, namely Extracted Ion Chromatography (EIC) using LC/UV-MS detection, was used.

For this arm, sample formulation was done directly in the vials, with different formulations containing the selected antioxidants, the RSV preF2 antigen and 6.25 μg of SP per vial. This ensured an equal amount of total MET from SP as from RSV preF2 (3.5 nmoles in both cases). Samples were then subjected to the spiking/lyophilization described above and stored at 7D37° C. prior to analysis.

Met343 Oxidation Ratio by LC-MS (Arm #2)

The oxidation ratio of Met343 residues of RSV preF2 in FC lyo was assessed by LC-MS on a selection of samples based on results of arm #1 and arm #3. FC lyo of arm #2 were stored in forced ageing conditions at 7D37° C. before analysis. Non-spiked samples were used as controls.

Impact on Purity by RP-HPLC (Arm #2′)

Antioxidants that showed the best results (lowest Met343 oxidation ratio) were then selected for FC lyo analysis by RP-HPLC. This was done with samples subjected to H₂O₂ spiking at 0 μM vs. 27 μM, in order to assess:
The visual impact on chromatograms
The impact on Purity values (ratio of main peak integration vs. the sum of all peaks)

This was done in parallel to SDS-PAGE characterization (same arm, same samples).

Impact on Conformation by SDS-PAGE (Arm #2′)

As described FC lyo which included only the most effective antioxidants, based on LC-MS results, were analyzed by SDS-PAGE in non-reducing and in reducing conditions, to establish if the addition of the antioxidant to the formulation had an impact on RSV preF2 conformation. This was done with 1 μg deposited protein and a silver staining procedure.

Results:

Effect of Hydrogen Peroxide on RSV PreF2 Antigen:

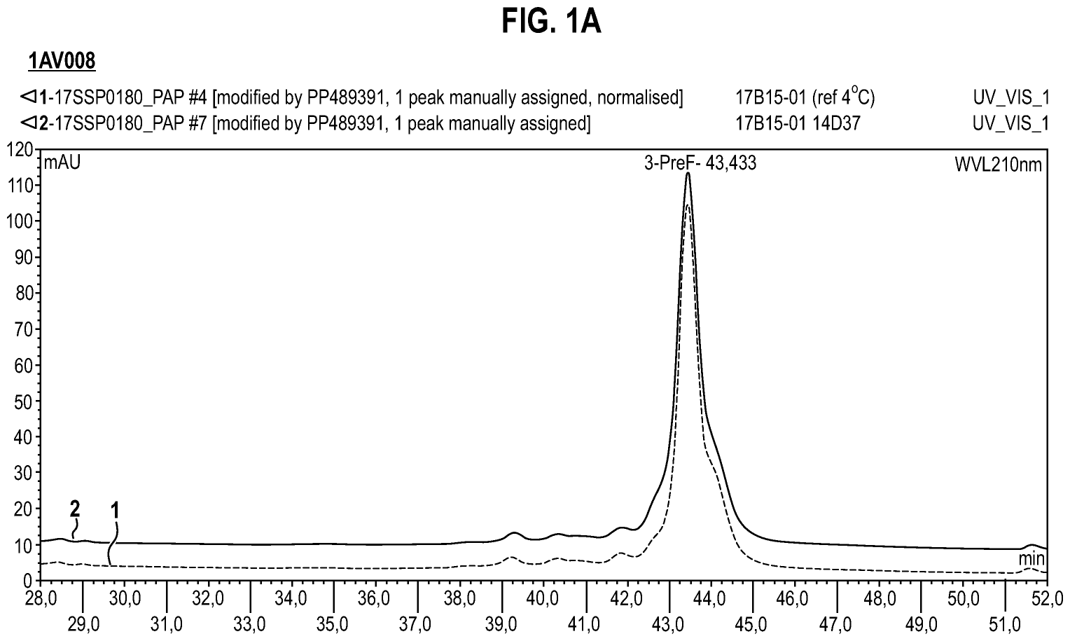

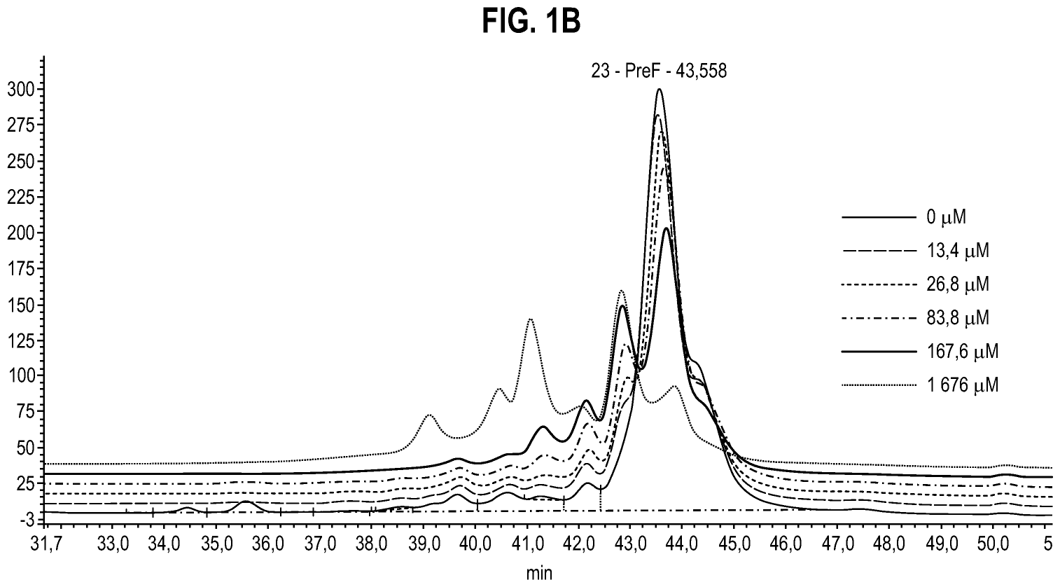

FIG. 1 shows representative RP-HPLC chromatograms as follows:

FIG. 1a: obtained for 0 μM spike between storage at 4° C. and at 14D37° C., showing that these storage conditions do not cause profile modification in samples not exposed to hydrogen peroxide.

FIG. 1b: obtained for 0 μM spike, 13.4 μM spike, 26.8 μM spike, 83.8 μM spike, 167.6 μM spike and 1676 μM spike, FC lyo after storage at 7D4° C. showing profile modification, dependent on the spiked concentration of hydrogen peroxide.

Antioxidant Potency and Impact on Conformation

H₂O₂ Consumption in the Presence of Antioxidants (Arm #1)

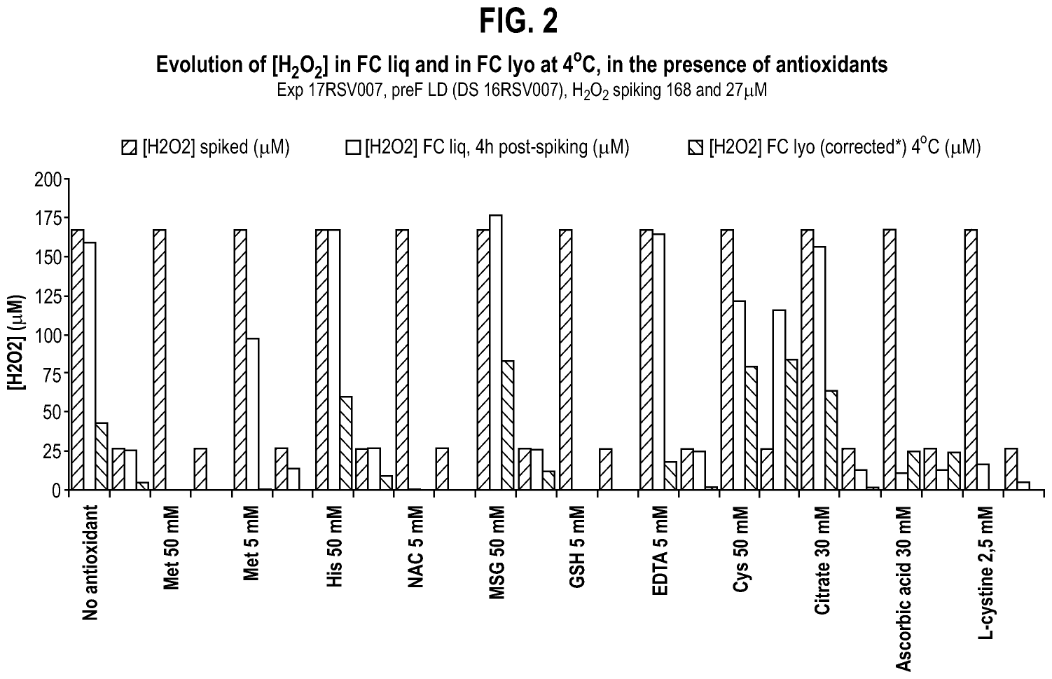

FIG. 2 shows evolution of [H₂O₂] in FC liquid 4 h post-spiking and in FC lyo after 4° C. storage in the absence and presence of different antioxidants, following H₂O₂ spikings of 168 and 27 μM.

As shown in FIG. 2, FC lyo were produced in order to compare the antioxidant potency of the different selected excipients at this step. There were 12 different antioxidant conditions (including 1 no antioxidant and 2 different concentrations of MET) added to the FB formulations including the RSV preF2 antigen, which were then spiked with 0, 27 and 168 μM of H₂O₂, respectively.

As shown in FIG. 2, in the case where no antioxidants were present in the formulation:
The amount of H₂O₂ found 4 h after spiking was the same as the amount initially spiked, considering analytical variability.
The same observation was made for the lower (27 μM) and higher (168 μM) H₂O₂ spikes.
Following lyophilization, (assessed after 10D4° C. storage of the FC lyo) a decrease of \tilde{ }75% of the H₂O₂ content was observed in the reconstituted vaccine (RV).
Comparable ratios were observed for the lower and higher H₂O₂ spikes.
Note: we know from previous experiments (results not shown), that after storage at 37° C. for 7 days, close to no remaining H₂O₂ was found in FC lyo.

In the presence of some of the antioxidants (MET, NAC, GSH, Ascorbic acid, L-Cystine) the H₂O₂ amounts found after lyophilization (FC lyo bars adjusted to take a 1.25-fold dilution factor into account) were lower than in the no antioxidant control group.

In the case of MET at the highest 50 mM concentration and NAC 5 mM, complete H₂O₂ consumption was already observed before the lyophilization step.

In the case of MET at the lowest 5 mM concentration and L-Cystine 2.5 mM a partial H₂O₂ consumption was already observed before the lyophilization step.

This indicates that MET, NAC and L-Cystine were potent enough to consume H₂O₂ in a short 4 h timeframe in FC liquid, before performing the lyophilization, which is known to induce a critical cryoconcentration step.

Some samples (i.e. CYS) showed a higher H₂O₂ content following spiking than the amount spiked, which was explained by interference with the analytical testing (results not taken into account at this step). Moreover, the analysis of the absorbance of blanks (data not shown), using samples containing antioxidant and not spiked with H₂O₂ showed that the presence of some antioxidants in the FC liq could lead to a very high blank absorbance. This shows that the analysis of these samples was unreliable, especially considering that the calibration curves were all obtained from H₂O₂ standards diluted in RSV preF2 buffer without the antioxidant. In particular, the results obtained for Citrate, 3Na and L-Cystine were discarded at this step.

In Conclusion:
5 candidates were selected based on their potency to protect RSV preF2 from oxidation by H₂O₂ and were classified as follows: NAC 5 mM=MET 50 mM=GSH 5 mM>MET 5 mM≅Ascorbic acid.
No improvement from the negative control could be observed for HIS and MSG in these experimental conditions.
Results were considered unreliable for CYS, Citrate 3Na and L-Cystine because of analytical interferences (high blank absorbance).

Oxidation Ratio of Substance P as a Model Protein as Assessed by LC/UV-MS

Substance P and 12 antioxidant conditions (including 1 no antioxidant and 2 different concentrations of MET) were added to the FB formulation, co-lyophilized with RSV preF2 and then spiked with 0, 27 and 168 μM of H₂O₂, respectively, then lyophilized after a 4 h hold-time using a standard 45 h lyophilization cycle. FC lyo were then stored under forced aging conditions at 7D37° C. and analyzed by LC/UV-MS to quantify the SP oxidation ratio.

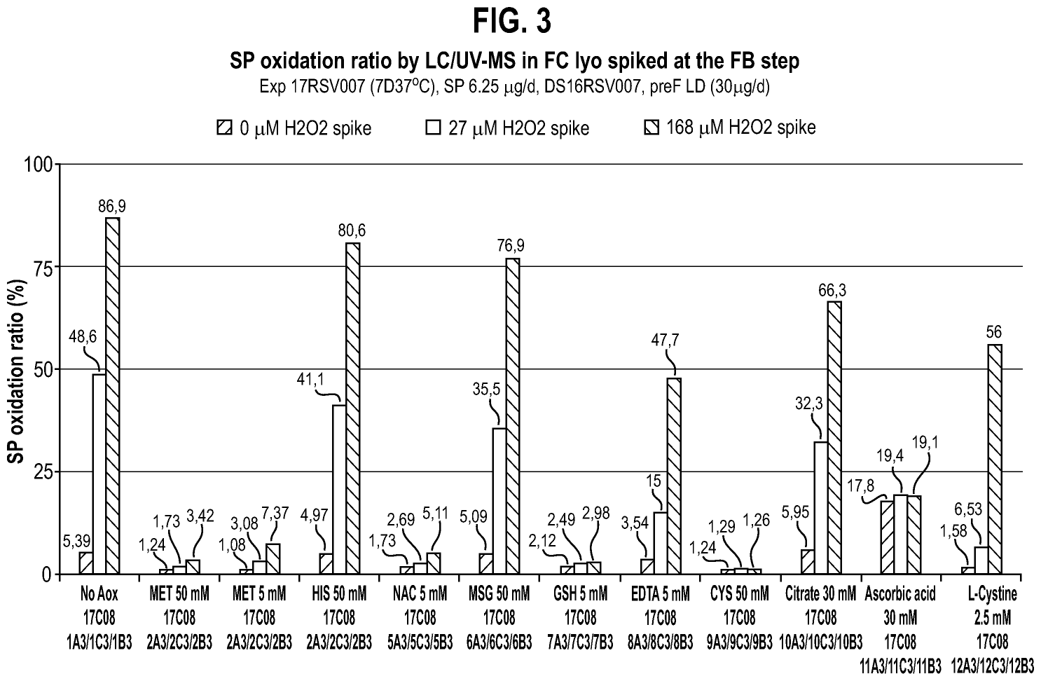

Results of this experiment (FIG. 3) show that:
Without antioxidant in the FB formulation SP had an oxidation ratio of:
   5.4% SP oxidation with a 0 μM spiking.
   48.6% SP oxidation with a 27 μM spiking.
   86.9% SP oxidation with a 168 μM spiking.
HIS 50 mM and MSG 50 mM were ineffective in protecting SP against oxidation from spiked H₂O_{2.}
EDTA 5 mM, Citrate 3Na 30 mM, Ascorbic acid 30 mM and L-Cystine 2.5 mM showed partial antioxidant potency against oxidation from spiked H₂O₂.
MET 5 and 50 mM, NAC 5 mM, GSH 5 mM and Cys 50 mM exhibited a very high protection against oxidation from spiked H₂O₂.
With a 0 μM spiking (no H₂O₂): the formulation/filling/lyophilization process seemed to cause baseline oxidation of SP in non-spiked samples (5.4% oxidized), preventable through the addition of the most potent antioxidant in the FB formulation:
   MET 5 and 50 mM: 1.08 and 1.24% SP oxidation
   NAC 5 mM: 1.73% SP oxidation
   GSH 5 mM: 2.12% SP oxidation
   CYS 50 mM: 1.24% SP oxidation
   L-Cystine 2.5 mM: 1.58% SP oxidation
With a 27 μM H₂O₂ spiking, from a negative control of 48.6% SP oxidation, the addition of the most potent antioxidant prevented SP oxidation:
   MET 5 and 50 mM: 3.08 and 1.73% SP oxidation
   NAC 5 mM: 2.69% SP oxidation
   GSH 5 mM: 2.49% SP oxidation
   CYS 50 mM: 1.29% SP oxidation
   L-Cys 2.5 mM: 6.53% SP oxidation
With a 168 μM H₂O₂ spiking, from a negative control of 86.9% SP oxidation, the addition of the most potent antioxidant prevented SP oxidation:
   MET 5 and 50 mM: 7.37 and 3.42% SP oxidation
   NAC 5 mM: 5.11% SP oxidation
   GSH 5 mM: 2.98% SP oxidation
   CYS 50 mM: 1.26% SP oxidation
L-Cystine did not prevent SP oxidation sufficiently under this spiking (56% SP oxidation) Ascorbic acid 30 mM gave mixed results, increase of SP oxidation (17.8%) at 0 μM spiking and comparable levels under 27 and 168 μM spikings (19.4 and 19.1% SP oxidation) which could have been caused either by analytical interferences or by a reversible oxidation process at the equilibrium, showing both antioxidant and pro-oxidant properties of this excipient.

In conclusion, the following selection of antioxidants based on their potency against SP oxidation could be made: CYS 50 mM>MET 50 mM>GSH 5 mM>NAC 5 mM>MET 5 mM.

This classification confirms the previous results obtained regarding the evolution of the H₂O₂ content, except for CYS, which was previously left out as it interfered with the HRP analytical assay.

Ascorbic acid was also maintained further in the screening as the results observed with both methods could be the result of analytical interferences.

Oxidation as Assessed by LC-MS (Met343Ox Ratio)

Based on the previous observations, only MET 50 mM, MET 5 mM, NAC 5 mM, GSH 5 mM, Ascorbic acid 30 mM spiked at the more representative condition of 27 μM H₂O₂ were analyzed against a 0 μM spike. FC lyo were stored under forced aging conditions at 7D37° C.

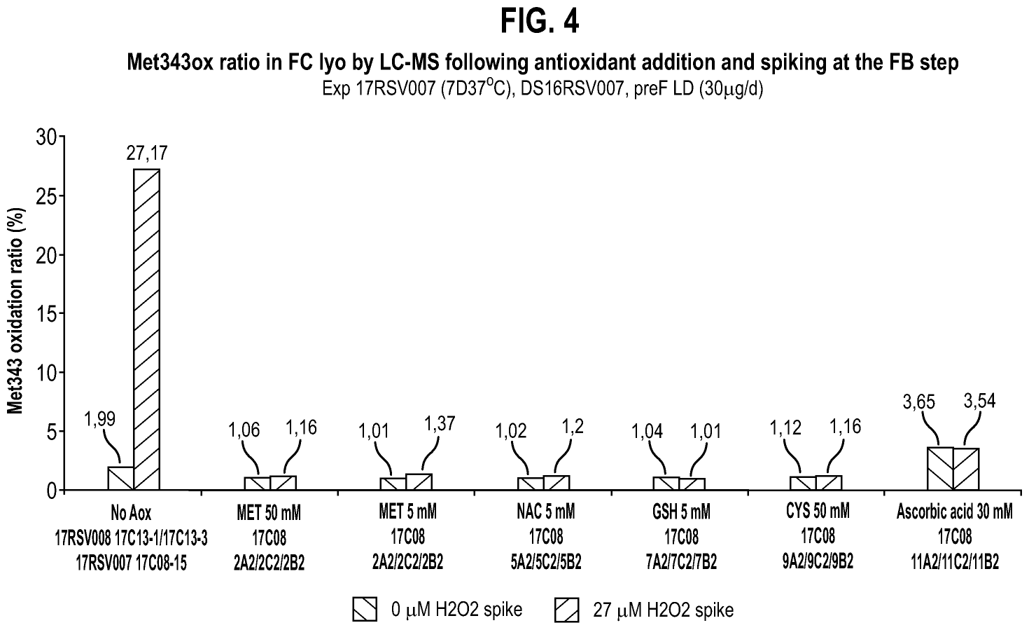

The screening shown in FIG. 4 shows:
With a 0 μM spiking (no H₂O₂): the formulation/filling/lyophilization process seemed to cause baseline oxidation of RSV preF2 in non-spiked samples (1.99% oxidized), preventable through the addition of the most potent antioxidant in the FB formulation:
   MET 5 and 50 mM: 1.01 and 1.06% Met343Ox
   NAC 5 mM: 1.02% SP oxidation
   GSH 5 mM: 1.04% SP oxidation
   CYS 50 mM: 1.12% SP oxidation
With a 27 μM H₂O₂ spiking, from a negative control of 27.17% Met343 oxidation, the addition of the most potent antioxidant prevented RSV preF2 oxidation:
   MET 5 and 50 mM: 1.37 and 1.16% SP oxidation
   NAC 5 mM: 1.2% SP oxidation
   GSH 5 mM: 1.01% SP oxidation
   CYS 50 mM: 1.16% SP oxidation
MET 50 and 5 mM, NAC 5 mM, GSH 5 mM and CYS 50 mM are protective of RSV preF2 oxidation from a 27 μM spike of H₂O₂
The experiment confirmed that Ascorbic acid 30 mM had both antioxidant and pro-oxidant properties as it showed:
   A higher Met343Ox ratio than negative control against a 0 μM H₂O₂ spike (3.54% Met343Ox)
   A lower Met343Ox ratio than positive control against a 27 μM H₂O₂ spike (3.54% Met343Ox)
It also confirmed that oxidation related to the formulation/filling/lyophilization process (e.g. oxidation by air) was preventable by the addition of antioxidant (0 μM spike without antioxidant: 1.99% Met343ox ratio vs. ±1.0% after a 0 μM spike with the most potent antioxidants).

It should be noted that this assay is destructive and was therefore only able to quantify the oxidized Methionine ratio of a specific peptide resulting from enzymatic digestion (i.e. IMTSK peptide). Therefore it did not give information on the impact of oxidation or of the addition of antioxidants on the overall RSV preF2 structure.

Oxidation and Impact on RP-HPLC Chromatograms

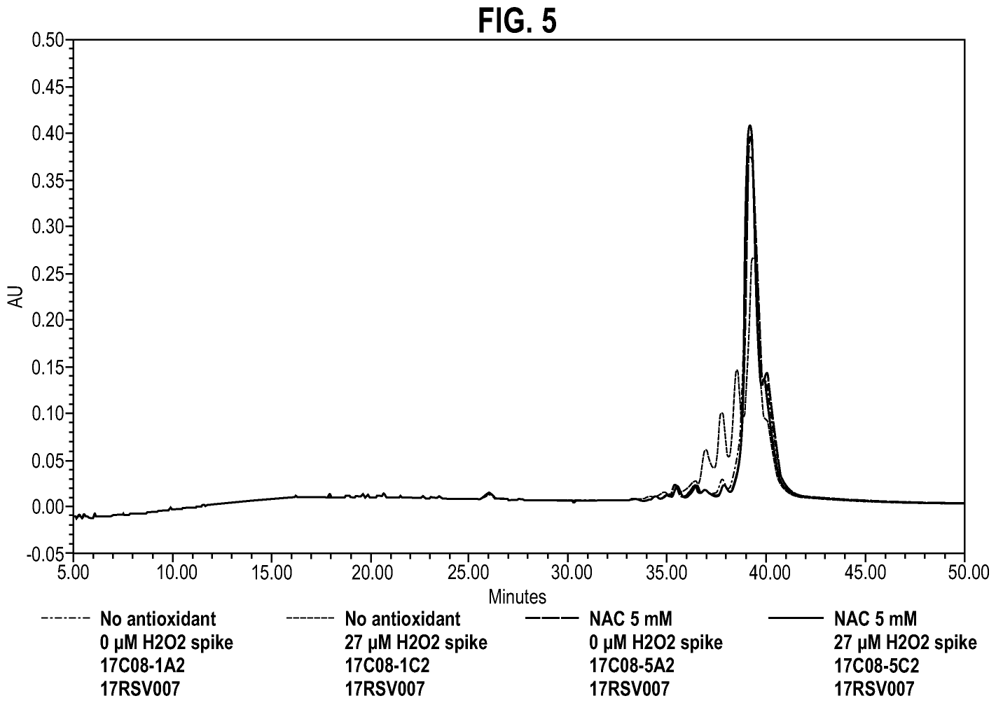

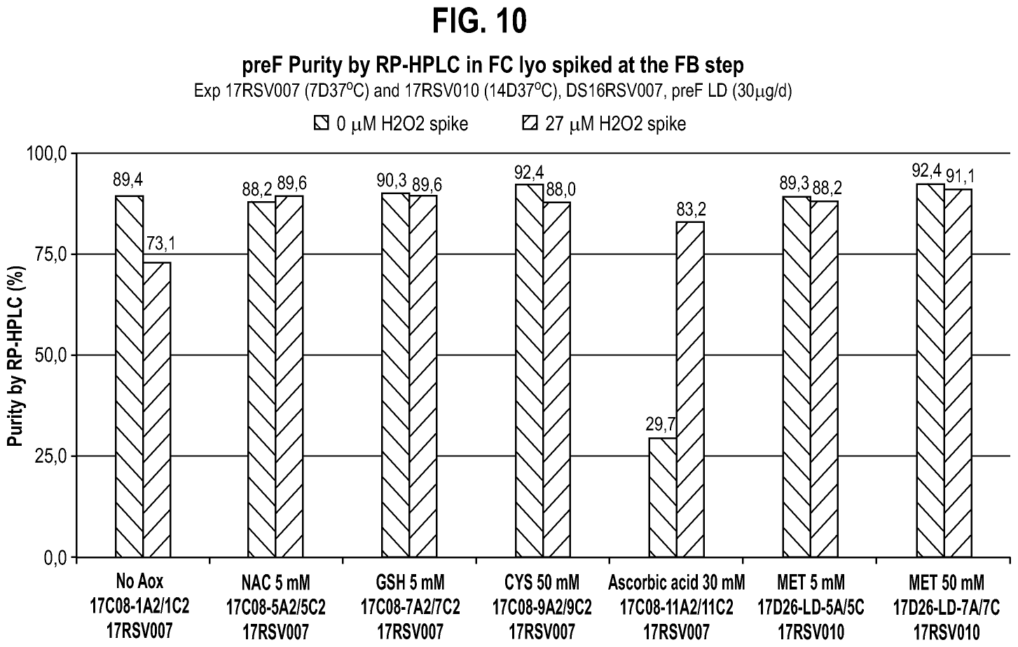

In order to determine if RSV preF2 oxidation impacts the purity read-out by high-resolution RP-HPLC and to determine if that can be avoided by using antioxidants, the same conditions as those analyzed by LC-MS (Met343ox ratio) were analyzed by RP-HPLC. FC lyo were stored under forced aging conditions at 7D37° C. The Chromatograms are shown in FIGS. 5-9 and discussed below. Results comparing the different potential antioxidants for this readout are shown in FIG. 10.

The analysis of the qualitative of the chromatograms with the basal, non-spiked profiles (in black) and the 27 μM spiked profiles (in light grey) is shown in FIGS. 5-9. This analysis shows the notable impact of the 27 μM spiking on the profile compared to a 0 μM spiked negative control.

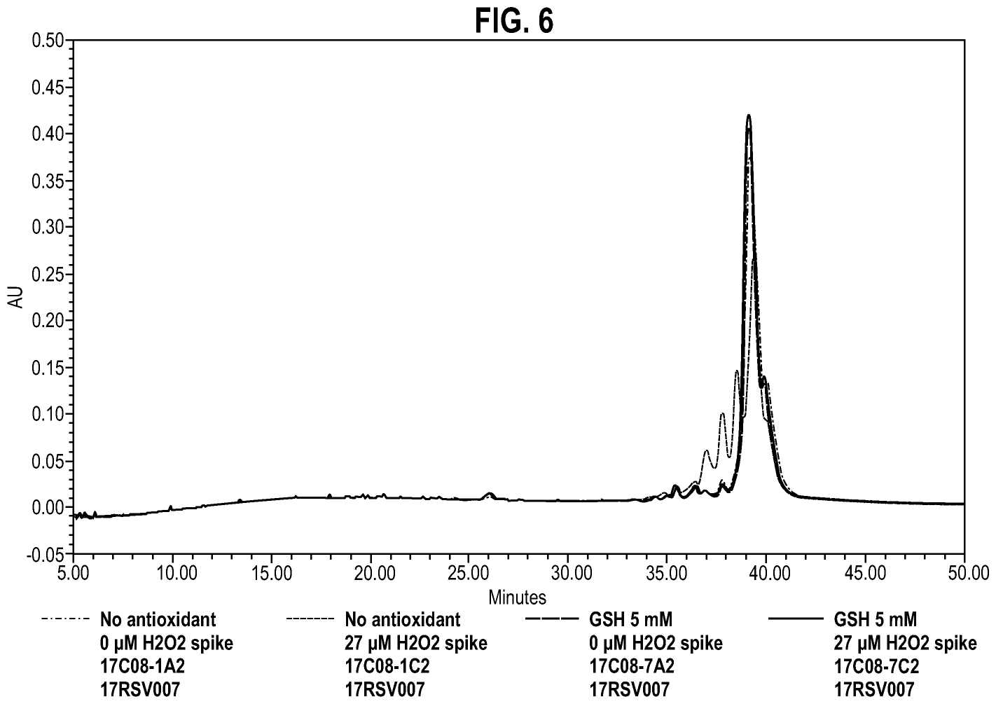

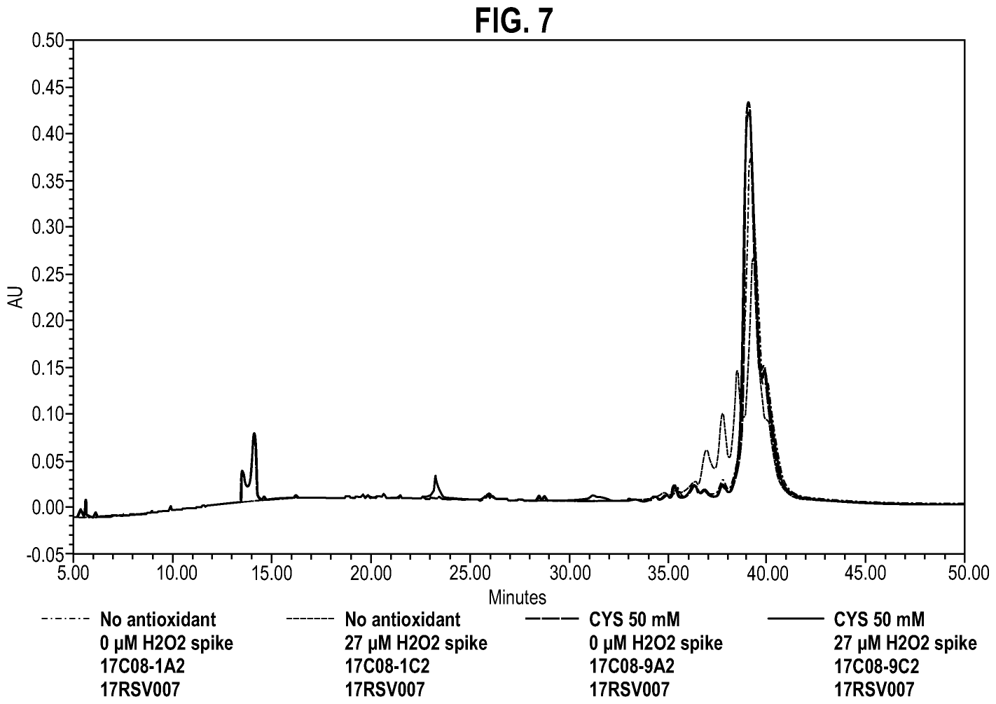

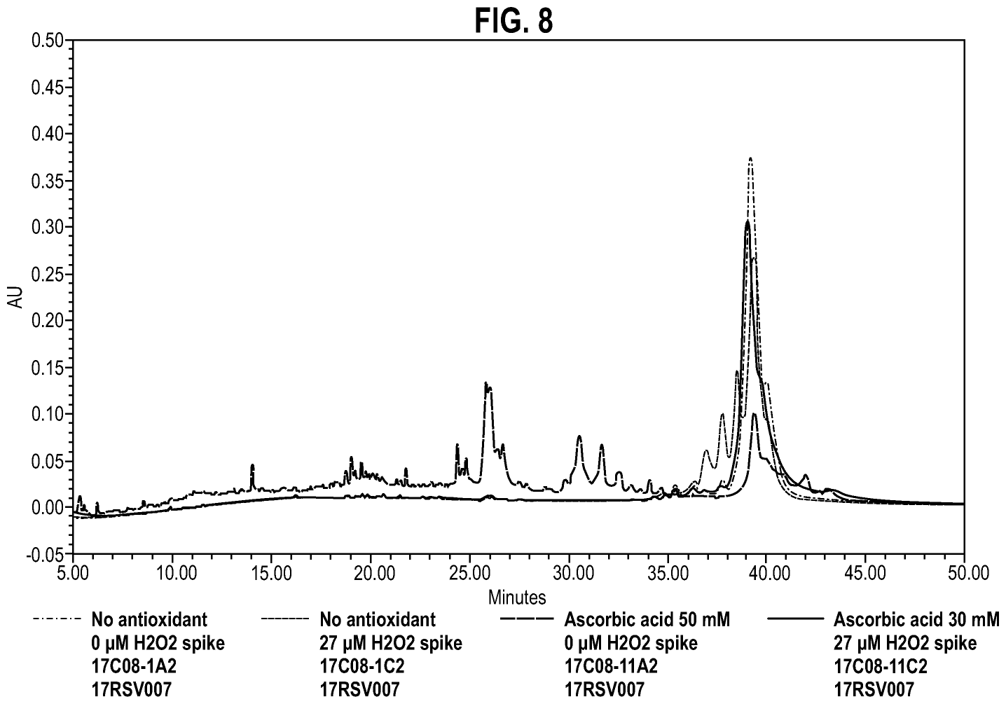

The antioxidant conditions showed:
NAC 5 mM (FIG. 5) and GSH 5 mM (FIG. 6) showed no impact of the antioxidant addition on RSV preF2 when spiked with 0 μM H₂O₂ and very good protection when spiked with 27 μM H₂O₂.
CYS 50 mM (FIG. 7) showed the appearance of new small hydrophilic peaks, eluted between 13 and 15 minutes but altogether little impact of the antioxidant addition on RSV preF2 when spiked with 0 μM H₂O₂ and very good protection ability of the main peak following a 27 μM H₂O₂ spiking.
Ascorbic acid 30 mM (FIG. 8) showed mixed results, with a very high impact with 0 μM H₂O₂ and improvement in the presence of a 27 μM H₂O₂ spike. This confirms the ambivalent behavior of this antioxidant.
MET 5 and 50 mM (FIGS. 9a and 9b) showed no impact of the antioxidant addition on RSV preF2 when spiked with 0 μM H₂O₂ and very good protection when spiked with 27 μm H₂O₂.

The analysis of Purity as the ratio of the main peak integration to the integration of all peaks in the chromatograms is given in FIG. 10, with the evolution of RSV preF2 purity by RP-HPLC in FC lyo spiked at the FB step with 0 or 27 μM H₂O₂ with regard to the antioxidants selected.

It showed that from an impact of a 27 μM H₂O₂ spike, lowering purity from 89.4% to 73.1%, the addition of the most potent antioxidants in the formulations (NAC 5 mM, GSH 5 mM, CYS 50 mM, MET 5 mM and 50 mM) was able to maintain the high degree of Purity of the RSV preF2 antigen (>88.0%). Ascorbic acid 30 mM showed once-again mixed results with pro-oxidant activity in absence of H₂O₂ and protective effect under 27 μM H₂O₂ spike.

It should be noted that this assay was performed after sample preparation in denaturing and reducing conditions (sodium dodecyl sulfate SDS 1%, dithiothreitol DTT 32 mM) and was therefore unable to detect alteration to the quaternary or tertiary structure of the protein.

Impact on Conformation by SDS-PAGE

The same samples as those selected for RP-HPLC were analyzed by SDS-PAGE, in reducing and in non-reducing conditions using β-mercaptoethanol as a reducing agent and silver-staining for detection. In addition, impact of oxidation was assessed using internal controls (DS, FC spiked at 0, 27 and 168 μM H₂O₂ at the FB step, Wells #1 to #4 and #11 to #14). Except for the DS (well #1) all FC lyo samples had been subjected to forced aging at 7D37° C. prior to analysis.

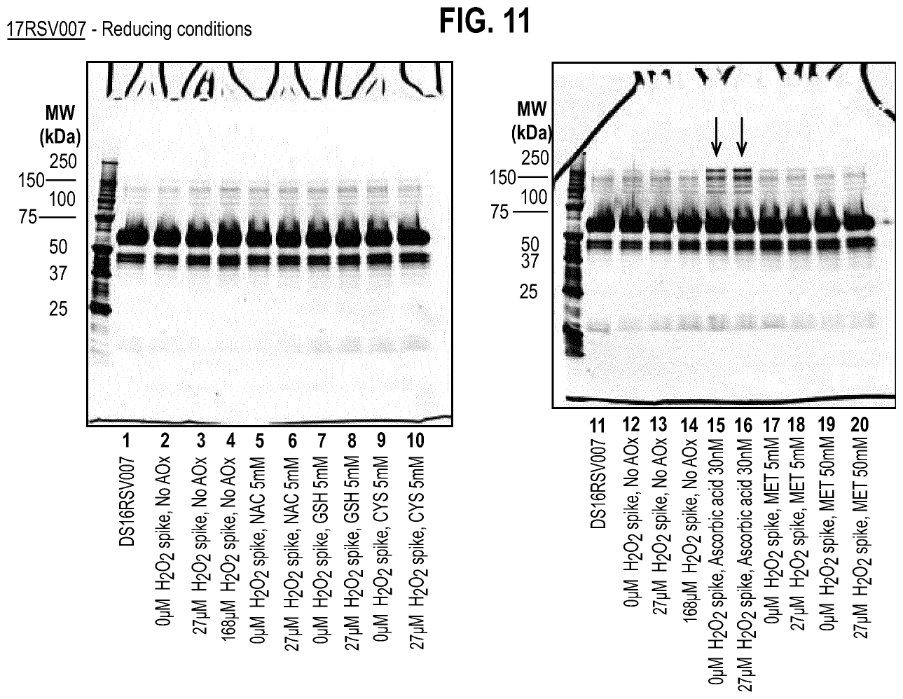

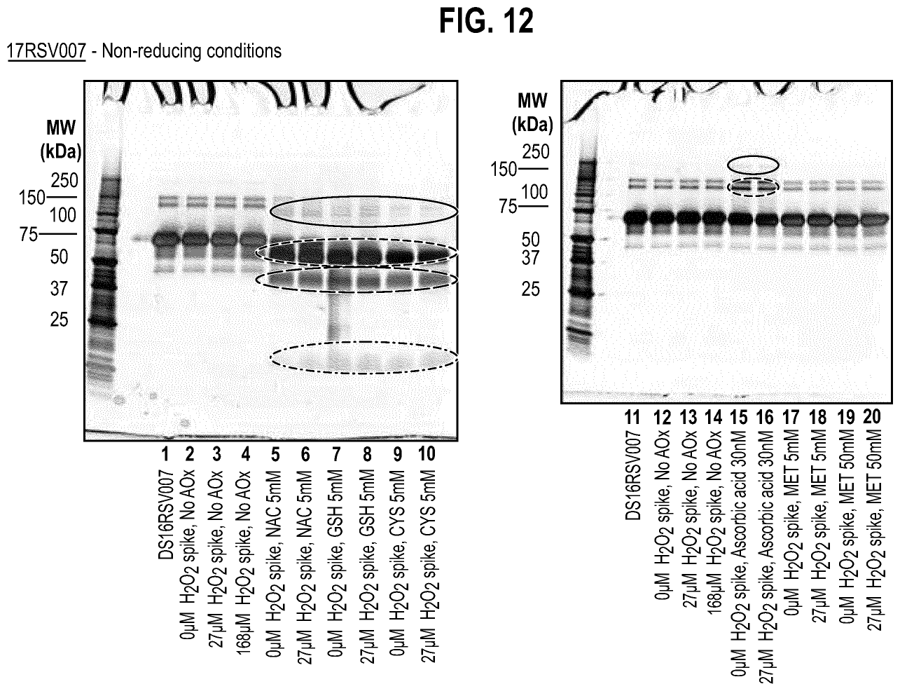

As shown in FIG. 11 and FIG. 12, 27 and 168 μM H₂O₂ spiking had no visible impact on the oxidation of RSV preF2 in FC lyo. As a consequence, further impact on SDS-PAGE in non-reducing and in reducing conditions can only be linked to modification in the protein structure upon antioxidant addition and not to RSV preF2 oxidation.

NAC 5 mM (Wells #5 and #6), GSH 5 mM (Wells #7 and #8) and CYS 50 mM (Wells #9 and #10) showed no visible impact in reducing conditions (FIG. 11). However, in non-reducing conditions (FIG. 12) a molecular weight decrease of the higher order structure from \tilde{ }150 kDa to the \tilde{ }120 kDa region was clearly observed. Regarding protein sub-units, clear modifications are visible with the main original peak at \tilde{ }70 kDa as seen in controls, split in two peaks between \tilde{ }50 kDa and \tilde{ }40 kDa, regardless of H₂O₂ exposure.

All the thiol-based (R—S—H) antioxidants (NAC, GSH, CYS) screened showed a very clear modification of the native SDS-PAGE profile obtained in non-reducing conditions, with profiles comparable with those observed in non-reducing conditions. By definition, antioxidants are reductive species and the presence of thiols with strong reducing properties in the formulation could therefore be responsible for the alteration of disulphide bonds in the native RSV preF2 protein. Deprotonated thiols (thiolates) are known nucleophiles and, depending on the conditions (pKa, nucleophilicity), often result in the attack of existing disulphide bonds.

Ascorbic acid 30 mM (Wells #15 and #16) showed comparable modifications in both reduced and non-reduced conditions. In both cases, the higher order structure related peak at \tilde{ }150 kDa appears more intense than in controls. No modification can be seen regarding the molecular weight of migrated peaks. No impact can be observed between formulations exposed and not exposed to H₂O₂ conditions.

Methionine 5 and 50 mM (Wells #17 and #18 and #19 and #20, respectively) was the only antioxidant assessed showing no modification of the molecular weight of migrated peaks nor of the peak intensity. No impact of oxidation could be observed either.

In conclusion, RSV preF2 structure analysed by SDS-PAGE was affected by the presence of thiol-based antioxidants (NAC, GSH, CYS), which are strong reducing agents. Their use was therefore not acceptable in the RSV preF2 formulation as they would alter the conformation and potentially the immunogenic profile of the antigen. Methionine, a less reactive thioether antioxidant was the best approach.

Conclusion:

Methionine is the best suited antioxidant for RSV preF2 against oxidation by residual VHP and by air during lyophilization. It has the further advantages that:
It is approved by the FDA as an inactive ingredient
It is present in marketed injectable products at concentrations up to 15 mM
Its toxicity is very well-characterized
   It has inherently low toxicity as it is an amino-acid
   It shows very low acute (high LD₅₀) and chronic toxicity (high No Observed Adverse Effect Level)
It showed potent antioxidant activity against H₂O₂ spikes representative of residual VHP at concentration of 5 and 50 mM in FB (4 and 40 μM in RV).
   Through direct H₂O₂ consumption (in FB and in FC lyo)
   Through direct measurements
      On a model protein (SP)
      On RSV preF2 by Methionine oxidation
      On RSV preF2 by protection of the chromatographic profiles observed by RP-HPLC
It showed no impact on protein conformation assessed by SDS-PAGE, unlike all the other antioxidants screened

A dose-definition study was carried out using different concentrations of H₂O₂ spiking and ultimately VHP in order to select the ideal concentration of antioxidant in RSV preF2 formulations (see Example 2).

### Example 2

### Dose Ranging Study to Determine Optimum Concentration of Methionine for Protection of RSV preF2 Against Oxidation

Introduction

Following Example 1 in which the most suited antioxidant was determined to be MET, this experiment focused on determining the best concentration to add to the FB formulation of RSV preF2 through a dose-range study followed by representative process including HP spiking to mimic residual VHP exposure.

Methods

Formulation

The RSV preF2 amounts that were tested were:
A low antigen dose LD (same as in Example 1)
A mid antigen dose MD (2-fold higher than the low dose)
A high antigen dose HD (5-fold higher than the low dose)

The excipients that were in the formulation were in the same composition and proportion as in Example 1.

The MET amounts in the Final Bulk vaccine that were tested in this example ranged from:
0/0.05/0.075/0.1/0.125/0.150/0.175/0.2 mM for the production of samples ultimately spiked with 5 μM H₂O₂.
0/0.25/0.5/0.625/0.75/0.875/1 mM for the production of samples ultimately spiked with 44 μM H₂O₂.
0/0.125/0.875 mM for samples spiked with 0 μM H₂O₂ (blanks production).

The same production and evaluation process as with Example 1 was performed (formulation of a RSV preF2 FB with/without antioxidant, spiking, hold-time of 4 h, same lyophilisation cycle of 45 h as in Example 1, storage of FC under forced aging at 7D37° C.).

Regarding the H₂O₂ spiked in this dose-range study, the H₂O₂ concentration for spiking was increased to include wider margins, as shown in Table 3 below, but also at a lower H₂O₂ concentration, representative of a lower 0.1 ppm residual VHP.

TABLE 3
Key VHP concentrations (ppm) and corresponding
H₂O₂ (μM) used in this Example.

| | Corresponding representative |
|---|---|
| [VHP] isolator limits (ppm) | [H₂O₂] spiking (μM) |
| 0.1 | 5.0 |
| 1.0 | 44.0 |
| Higher concentration | 168.0 |
| non-representative of VHP isolator limit | |

Storage

Following lyophilisation, FC were stored at either 4° C. or at 37° C. for 7 days for accelerated stability studies. This duration was proven sufficient to reach the oxidation plateau by Met343Ox and by RP-HPLC.

Analytics

Analyses that were performed on the produced FC lyo were limited to those linked to oxidation. This was done considering that in Example 1, no impact on protein structure could be observed from oxidation or from MET addition.

The analyses performed were:
H₂O₂ quantification: FC 4° C. at RSV preF2 mid dose only
RP-HPLC (Purity): all samples, as a screening tool
LC-MS (Met343Ox): sample selection based on RP-HPLC results (LC-MS throughput constraints)

Additional measurements (basal Purity and Oxidation of the Drug Substance) were performed during this experiment in order to increase the number of controls at basal oxidation levels.

Results

HP Content in FC Lyo Stored at 4° C.

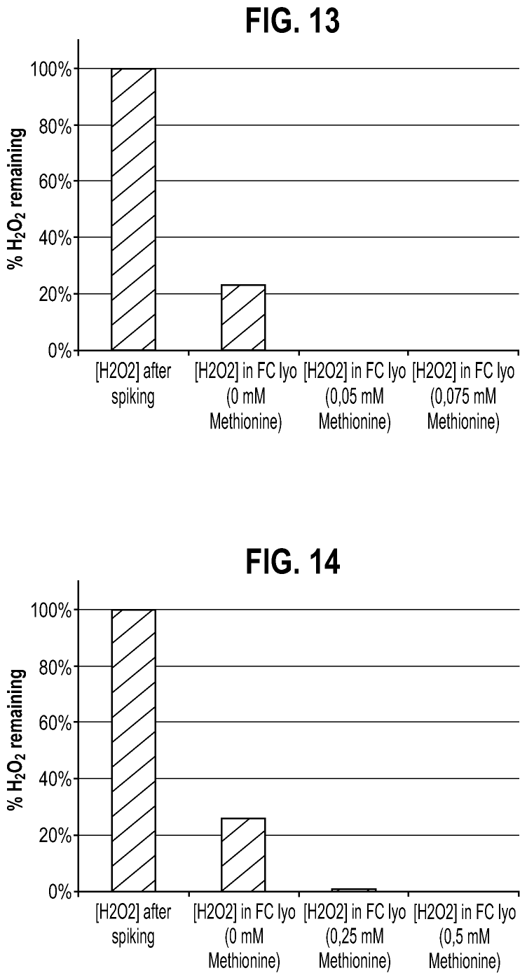

FIG. 13 shows a graphical representation of the effect of MET addition on H₂O₂ content in FC lyo in the case of a 5 μM spike.

In the case of samples spiked with 5 μM H₂O₂ representative of exposure to 0.1 ppm VHP:
H₂O₂ was only detected in samples containing no free MET, and was quantified at very low levels.
At levels of MET starting at 0.05 mM, no remaining H₂O₂ was found, while 20% average remaining H₂O₂ (between the H₂O₂ spiked in FB vs. H₂O₂ measured in FC lyo) was quantified in FC containing 0 mM MET.

FIG. 14 shows a graphical representation of the effect of MET addition on H₂O₂ content in FC lyo in the case of a 44 μM spike.

In the case of samples spiked with 44 μM H₂O₂ representative of exposure to 1.0 ppm VHP:
H₂O₂ was only detectable in the presence of 0.25 mM MET following a 44 μM H₂O₂ spike, with 0.29 μM H₂O₂ detected at the FC lyo step. This is equivalent to a 99.3% reduction in H₂O₂ content from the spiked concentration (vs. a lower 73.6% reduction at the same step in absence of MET).
For higher MET concentrations tested (0.5 mM and above), no H₂O₂ was found in FC lyo (100% reduction in H₂O₂ content from the spiked concentration.

In conclusion: H₂O₂ was totally eliminated from FC lyo in the presence of MET, even at the lowest concentration of:
0.05 mM when FB had been spiked with 5 μM H₂O₂
starting from 0.5 mM MET when FB had been spiked with 44 μM H₂O₂

Purity by RP-HPLC

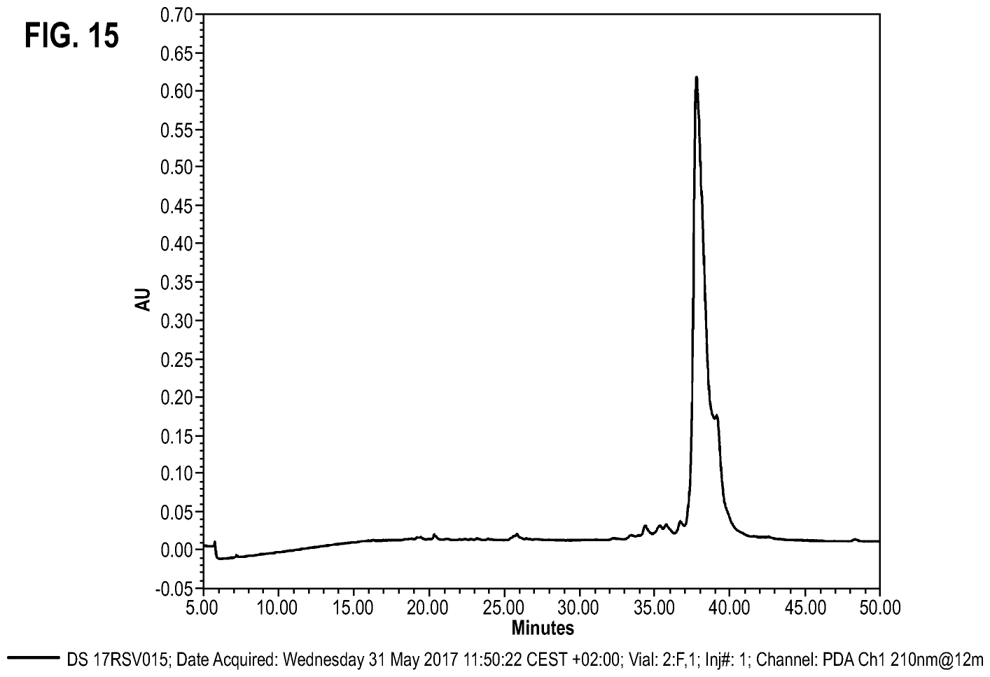

Following the same method as in Example 1, the purity of the Drug Substance lot used in this Example was used to establish a reference with a basal level of oxidation. The purity of the DS was established at a value of 91.77% (n=1). For reference, the obtained chromatogram is presented in FIG. 15.

This was followed by the analysis of the Purity by RP-HPLC of RSV preF2 in FC lyo following 4° C. and 7D37° C. storage. It showed that:
The RSV preF2 dose (low dose vs. mid dose vs. high dose) did not influence the purity measured at the FC lyo step after a 44 or a 5 μM H₂O₂ spikes in absence of MET, the major values were:
   Purity levels at 7D37° C. between 50 and 60% after a 44 μM H₂O₂ spike
   Purity levels at 7D37° C. between 80 and 85% after a 5 μM H₂O₂ spike
   vs. a 92% value in DS and in non-spiked FC.
The purity level after very short storage at 4° C. (<10 D) was not greatly affected as the oxidation is a relatively slow process in FC lyo under normal storage conditions
In the case of a 44 μM H₂O₂ spike, Purity was restored at levels of MET comprised between 0.625 mM and 0.75 mM, and the level of MET required was not linked to the RSV preF2 dose.
In the case of a 5 μM H₂O₂ spike, Purity was restored at a 0.075 mM level of MET, and the level of MET required was not linked to the RSV preF2 dose.
This exhibits a potential linear relationship between the H₂O₂ spiked concentration and the MET concentration needed in FB to control RSV preF2 purity.

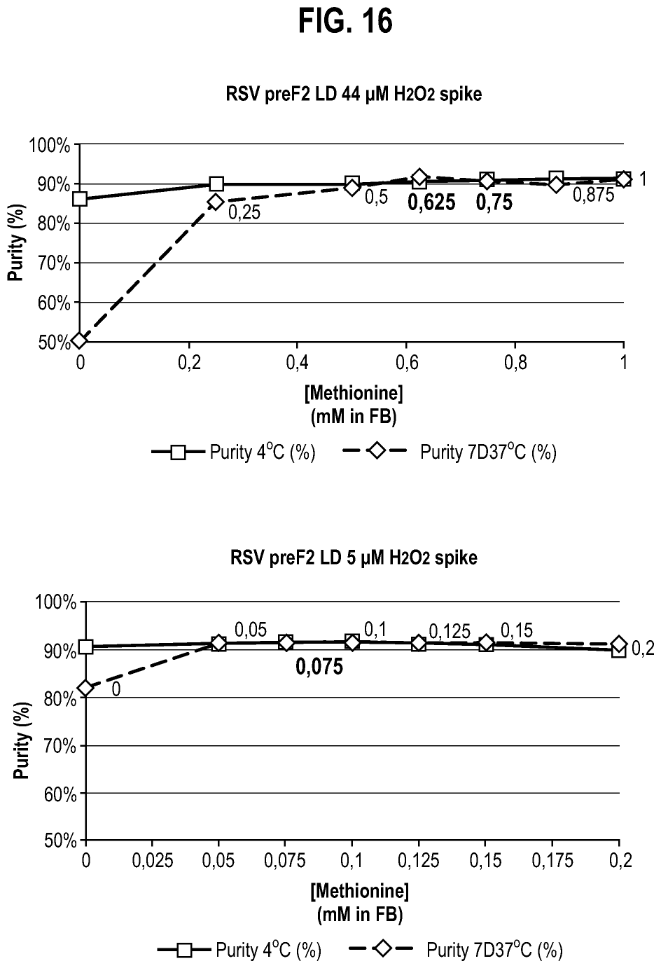

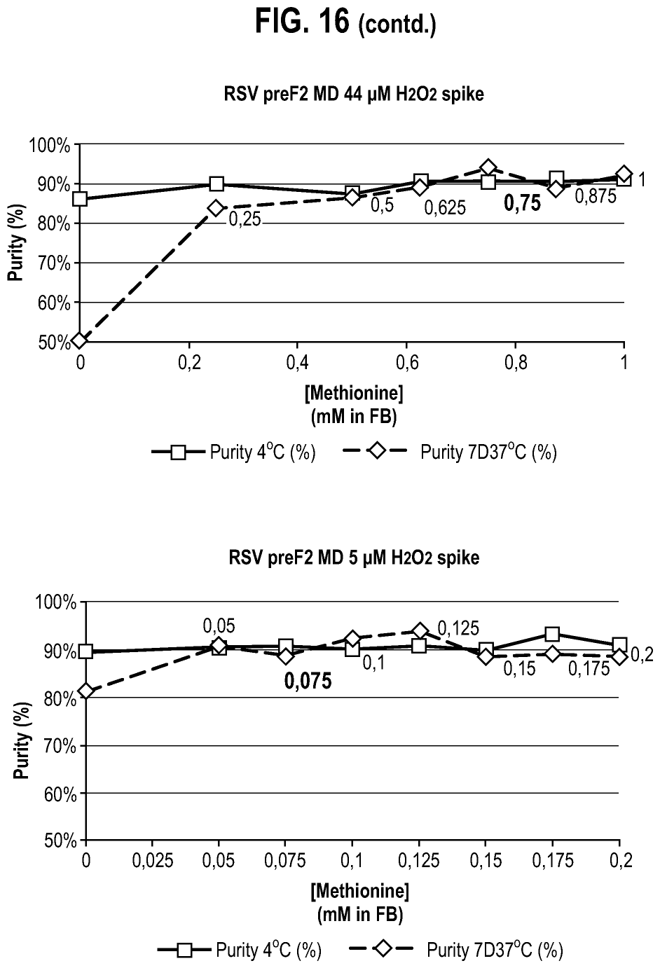

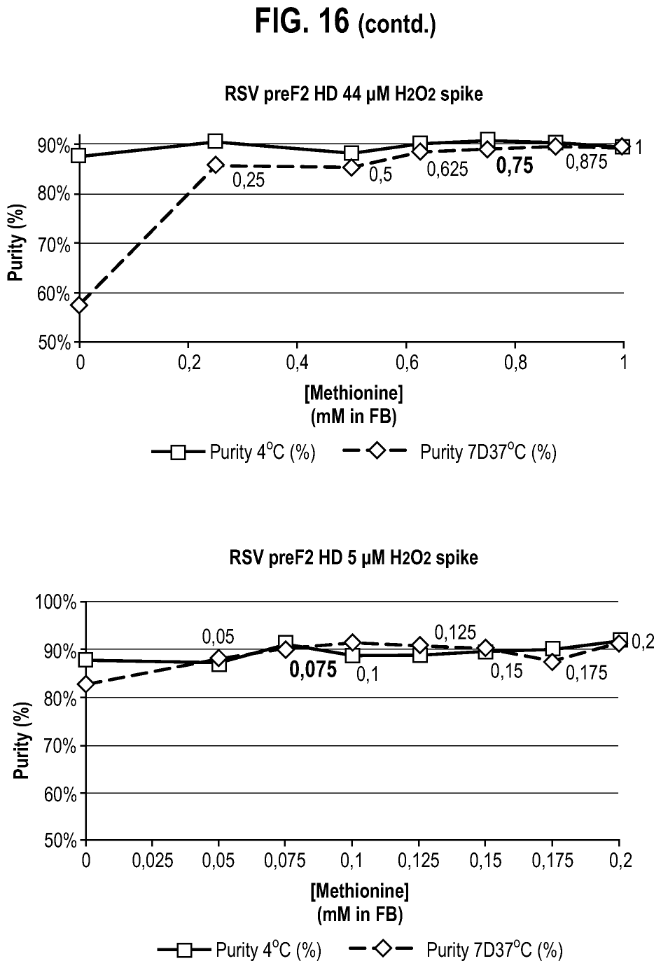

FIG. 16 shows evolution of RSV preF2 purity in FC lyo stored at 4° C. and 7D37° C. in the presence of increasing concentration of MET and following to 5 and 44 μM H₂O₂ spiking realized at the FB step.

In conclusion a level of MET of at least 0.625 mM for a 44 μM H₂O₂ spike, regardless of the antigen dose seemed fit to control the purity in this example. A level of MET of at least 0.075 for a 5 μM H₂O₂ spike seemed fit to control the purity by RP-HPLC in this example.

Met343Ox Ratio by LC-MS

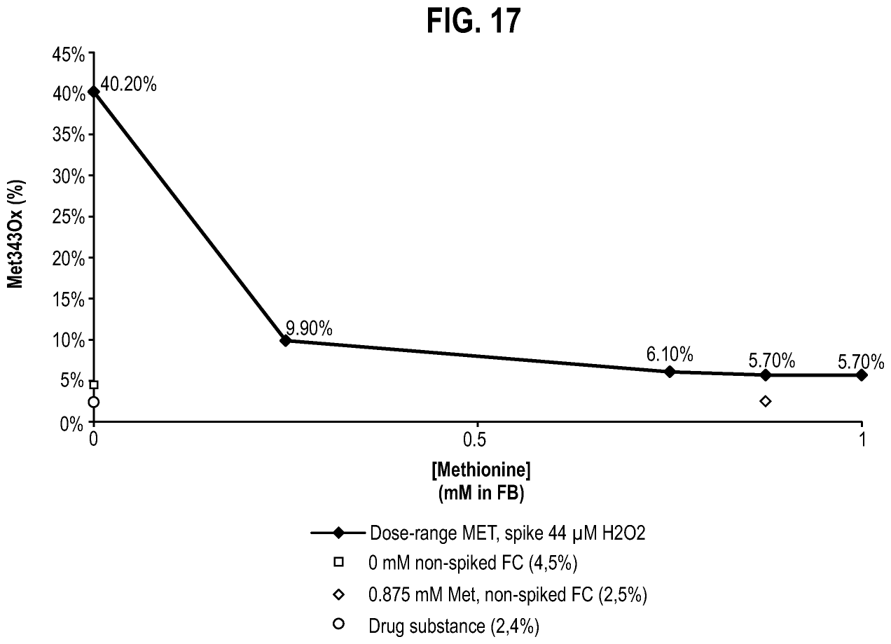

FIG. 17 shows evolution of Met343Ox ratio of FC, in relation to the Methionine concentration upon H₂O₂ spiking (at the FB step).

The analyses performed by LC-MS to determine the Met343Ox ratio of the RSV preF2 antigen, as was done in Example 1, showed that:
The DS lot used in this example exhibited a native oxidation ratio of 2.4% RSV preF2 Met343Ox.
A reference FC based on the same DS of RSV preF2 at LD, but spiked with 0 μM water exhibited an oxidation ratio of 4.6% Met343Ox (1.9-fold increase vs. the DS lot reference).
Samples spiked with 44 μM of H₂O₂and containing 0 mM MET exhibit a basal 40.2% RSV preF2 Met343Ox (8.7-fold increase vs. a non-spike reference FC)
Upon addition of 0.75 mM MET in the FB formulation prior to H₂O₂ exposure (an amount shown to be sufficient to control the impact on Purity), RSV preF2 Met343Ox was reduced to 6.1% RSV preF2 Met343Ox (1.3-fold increase vs. the non-spiked reference FC).
Upon further increase of the MET concentration (0.875 and 1.0 mM), the RSV preF2 Met343Ox ratio was further reduced to 5.7 and 5.7%, respectively (1.2-fold increase vs. the non-spiked reference FC).
The oxidation of the antigen, linked to the lyophilization process only (increase of Met343Ox levels between DS lot and non-spiked FC) was fully controlled by a MET addition of 0.875 μM—showing that the antioxidant addition is also effective in absence of H₂O₂.

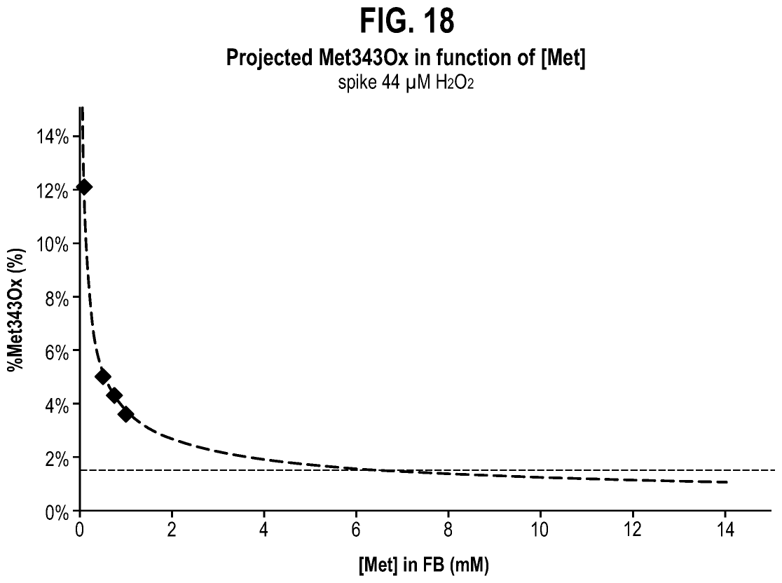

In the meantime, with data obtained from previous experiments, we showed that:
With a 44 μM spike and higher MET concentrations (2.0 mM), the Met343Ox ratio continued to decrease (3.6%), and the oxidation values of non-spiked FC value (3.3% in this case) were reachable. However, Met343Ox levels of the DS used to formulate (2.4%) were not reached using these levels of MET but the mathematical projection of the dose-range (FIG. 18) showed that a 6 mM MET would suffice to control the Met343Ox levels to 1.5%, back to DS lot oxidation levels.

General Conclusion

Oxidation assessed by LC-MS indicated the need for higher MET concentrations than what could be determined for RP-HPLC. While the latter indicated that a linear relationship seemed applicable for the control of Purity, this was not the case for oxidation assessed by LC-MS as the method is much more sensitive and specific to oxidation. In this case there was a saturation phenomenon for the efficacy of MET addition and the graphical projection seemed to follow a power decay, inferring for higher MET additions, comprised between 2 and 13 mM, depending on the level of oxidation control required.

The oxidation ratio of final container vaccine was directly linked to the oxidation ratio of the original drug substance. Furthermore, data showed that oxidation was taking place during lyophilization, even without H₂O₂, and that this phenomenon is controllable by MET addition.

### Example 3

### Antioxidants for a Composition Containing Protein D, PEPilA and UspA2

The sensitivity of the antigens present in a composition containing Protein D, PEPilA and UspA2 to oxidation by VHP was assessed.

It was demonstrated in the following experiments that methionine in Protein D is sensitive to oxidation, and in Protein D Methionine 192 is especially sensitive.

A first experiment consisted of spiking with liquid H₂O₂ at a range of concentrations: 0, 150, 800, 1300 and 5000 ng/mL. The vaccine batch which was not spiked with H₂O₂ (0 ng/mL) corresponded to the reference, to generate non-stressed, non-oxidized reference samples. Samples spiked at 150 and 1300 ng/mL were representative of the exposure for manufacturing at 0.1 and 1ppm v/v VHP in the isolator, respectively. The samples generated were then freeze dried and submitted to an accelerated stability plan at 25°, 37° C. and 45° C. and a real time stability at 4° C.

The impact of the H₂O₂ spiking was assessed by performing analytical tests after the different accelerated stabilities. Protein D was found to be the most sensitive antigen to oxidation, demonstrated by mass spectrometry. We observed high percentages of oxidized methionines and a molecular weight shift was observed by SDS page and in RP-HPLC chromatograms. A clear impact of the H₂O₂ level on the level of oxidized Met192 was observed; the higher the quantity of H₂O₂, the more Met192 was oxidized. Based on M192 oxidation, correlations could be established to determine the level of oxidation of the other methionines of Protein D, therefore M192 was used as a probe for oxidation. Furthermore, it was demonstrated that oxidation of M192 occurred even for an equivalent stress of 0.1 ppm v/v in manufacturing.

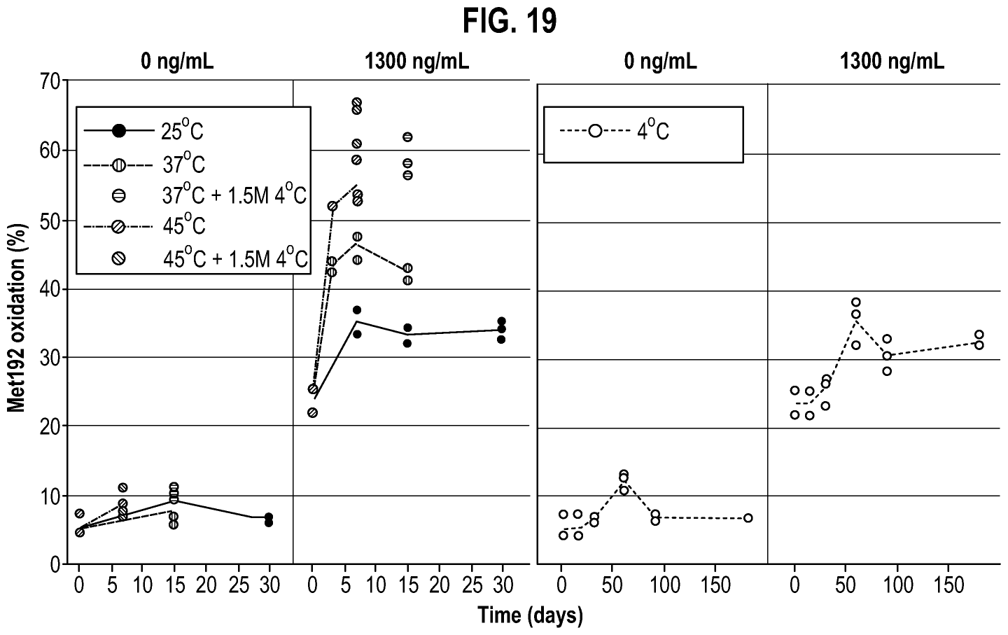

Results are shown in FIGS. 19 to 21 as follows.

FIG. 19 shows mass spectrometry results for protein D Met192 oxidation over time for 0 and 1300 ng/mL H₂O₂ at different temperatures. +/−55% oxidation is reached after 7 days at 45° C.

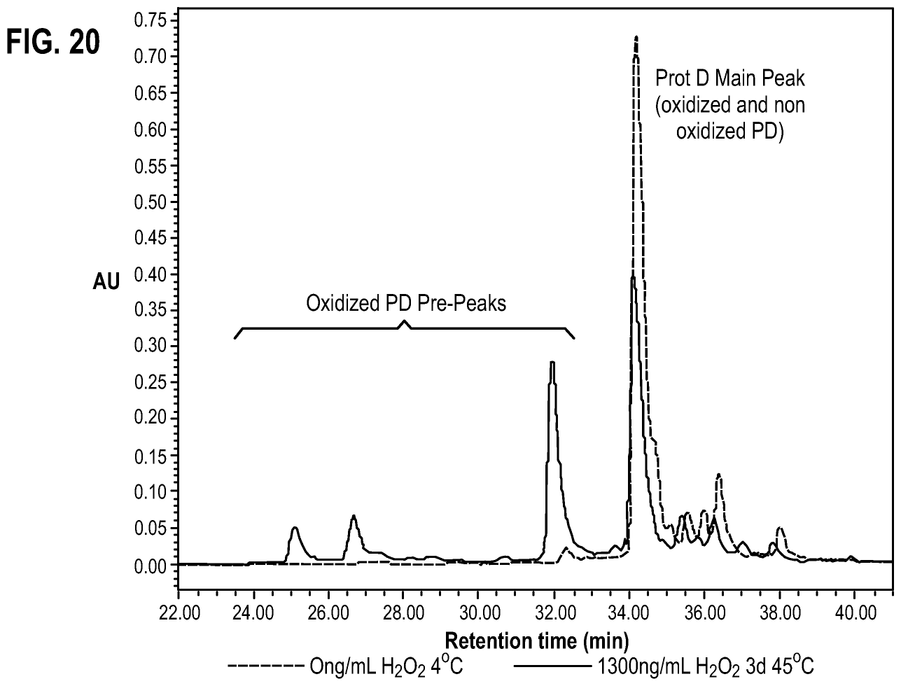

FIG. 20 shows a RP-HPLC chromatogram of oxidized protein D with 1300 ng/mL H₂O₂ stored for 3 days at 45° C. and of non-spiked protein D stored at 4° C.

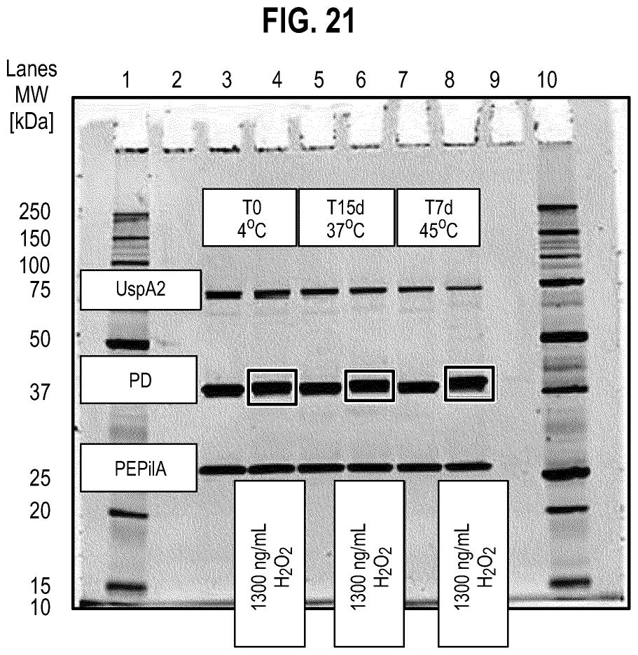

FIG. 21 shows antigen profiles obtained by SDS-PAGE in non-reducing conditions of samples, oxidized or not, stored at 4° C., for 15 days at 37° C. and for 7 days at 45° C. Lanes 4, 6 and 8 show oxidative stress impact on the protein D profile.

Assessment of Antioxidants

Experiments were designed to find out if the use of an antioxidant could prevent Protein D oxidation due to VHP oxidative stress encountered at manufacturing scale, and if so to determine which antioxidant would be most suitable.

Once again, the trivalent vaccine was spiked (or not) with H₂O₂ and then freeze dried. Formulations with and without L-methionine or cysteine were tested. Formulations contained either L-methionine at 50 mM or cysteine at 30 mM, prior to freeze drying.

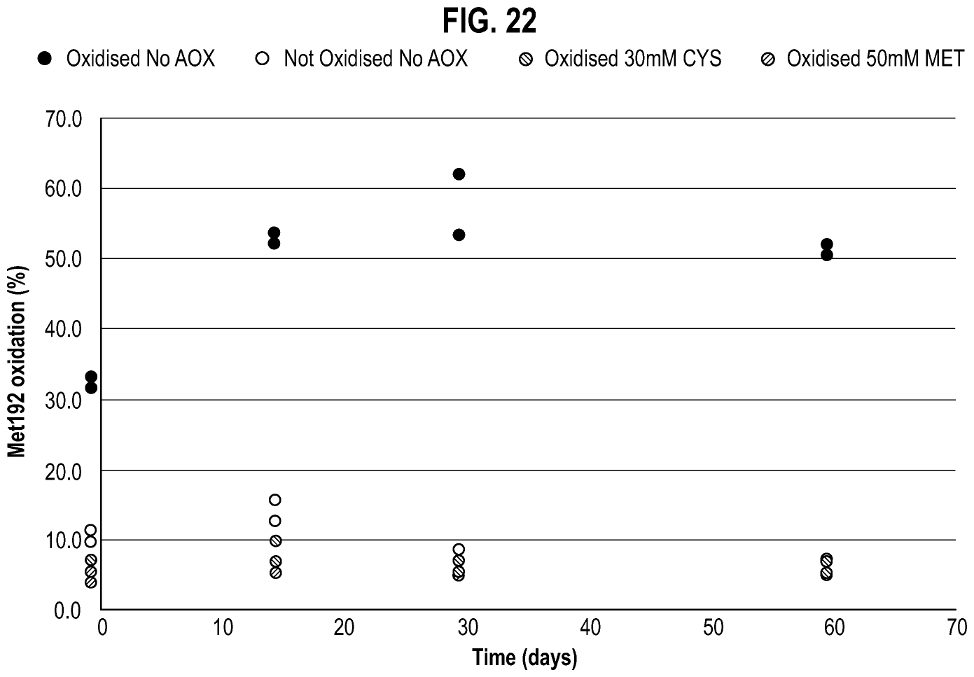

SDS-PAGE, hydrophobic variants RP-HPLC (which can also be referred to as purity by RP-HPLC) and Mass spectrometry were performed after 2 months at 37° C. on oxidized and non-oxidized samples containing either 50 mM methionine or 30 mM cysteine as antioxidant, or no antioxidant at all. Results are shown in FIGS. 22, 23 and 24.

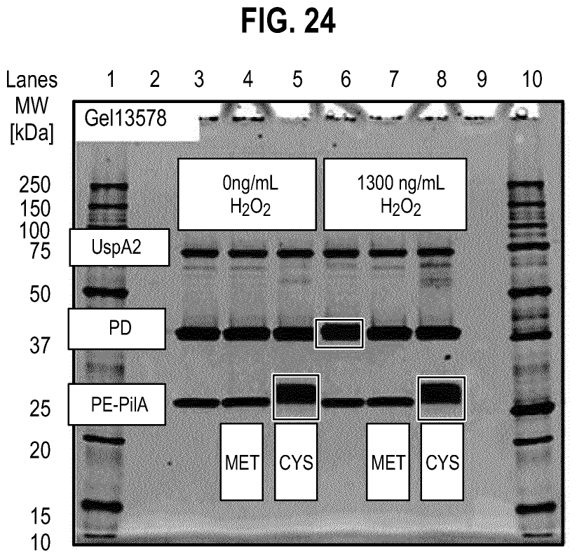

The antigen profiles obtained by SDS-PAGE in non-reducing conditions are shown in FIG. 24. Both cysteine and methionine prevented a molecular weight shift in protein D when samples were spiked with H₂O_{2.} Profile modifications of PE-PilA were observed in the presence of 30 mM cysteine. This was the case both for samples spiked with H₂O₂ and samples not spiked with H₂O₂. No profile modification was observed in the presence of methionine for the 3 antigens.

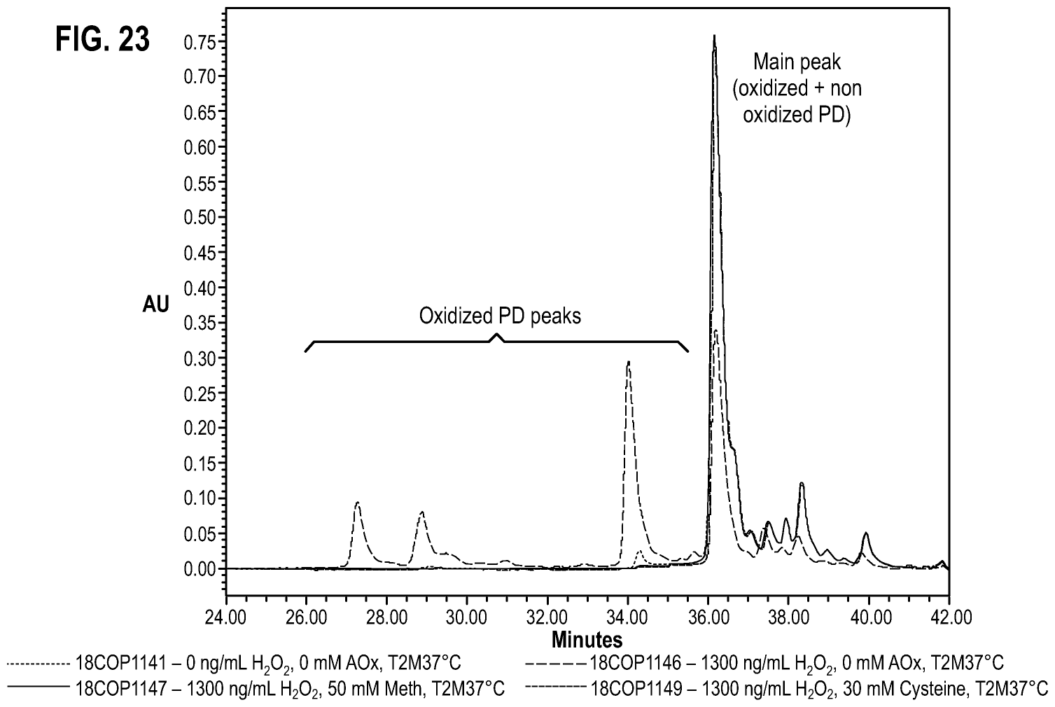

For the hydrophobic variants RP-HPLC, no profile modifications were observed in the presence of methionine for the 3 antigens compared to the non-oxidized reference sample. For cysteine no oxidation peaks were observed, though there was a decrease in Protein D main peak area, as for the H₂O₂ spiked control sample. The RP-HPLC chromatogram for protein D is shown in FIG. 23.

For the % methionine oxidation by mass spectrometry, antioxidant addition had a clear efficacy preventing oxidation for Protein D. The oxidation level in the presence of methionine was slightly lower than the oxidation level in presence of cysteine. No significant increase in oxidation was observed for PE-PilA or UspA2, in presence of H₂O₂, cysteine or methionine. The results for protein D only are shown in FIG. 22. Note that in FIG. 22 the 60 day results for samples with 50 mM methionine are not visible behind the dot representing 60 day results for samples with 30 mM cysteine.

Based on these results, methionine was identified as the most suitable antioxidant to protect against H₂O₂ mediated oxidation in this vaccine comprising Protein D, UspA2 and PE-PilA. Therefore, a methionine dose range experiment was performed to determine the exact methionine concentration that would be sufficient to prevent oxidation.

### Example 4

### Dose Ranging Study to Determine Optimum Concentration of Methionine for Protection of Protein D Against Oxidation

This Example shows RP-HPLC and mass spectrometry data that were generated to define the optimal L-methionine concentration to avoid oxidation of Protein D.

The optimal concentration of L-methionine as an antioxidant was determined by spiking 1300 ng of H₂O₂ per mL into compositions containing Protein D, PEPilA and UspA2, containing different concentrations of L-Met (Table 4 below). Subsequently the drug product was freeze dried and submitted to a stability plan (Table 5).

TABLE 4

| | Spiking [H₂O₂] | [MET] |
|---|---|---|
| ID Formulation | ng/mL | mM |
| 18COP1401 | 0 | 0 |
| 18COP1407 | 1300 | 0 |
| 18COP1402 | 1300 | 5 |
| 18COP1403 | 1300 | 10 |
| 18COP1404 | 1300 | 15 |
| 18COP1405 | 1300 | 25 |
| 18COP1406 | 1300 | 50 |

TABLE 5

| | Time | | | | | | | | | | |
|---|---|---|---|---|---|---|---|---|---|---|---|
| | T0 | T7 | T14 | T30 | T2 | T3 | T6 | T9 | T12 | T18 | T24 |
| Temp | days | days | days | days | months | months | months | months | months | Months | months |
| 4° C. | X | N/A | N/A | X | N/A | X | X | X | X | X | X |
| 37° C. | N/A | X | X | X | X | X | N/A | N/A | N/A | N/A | N/A |
| 45° C. | N/A | X | X | N/A | N/A | N/A | N/A | N/A | N/A | N/A | N/A |

The following tests were selected:
Hydrophobic variants by RP-HPLC:

3 vials per condition/time point; run of 54 min (specific to protein D) was applied for all samples except for batches 18COP1401, 18COP1402 and 18COP1407 after 15 days at 45° C. for which a run of 154 min (for 3 antigens) was applied; samples were randomized in the sample set;
Methionine oxidation (Met192 of Protein D) by mass spectrometry:

6 vials for batch 18COP1401 (reference sample), 18COP1403 (oxidized sample with 10 mM Met) and 18COP1407 (oxidized reference sample) after 1 month at 37° C. The sample containing 10 mM L-Met was selected for mass spectrometry analysis based on the RP-HPLC data for all samples after 7 and 14 days at 37° C. and 45° C.

The key objective of this experiment was to select the optimal concentration for L-Met as antioxidant to protect the drug product from oxidation. The optimal concentration of methionine assures an oxidation level for H₂O₂ spiked samples that is at least as good as a non H₂O₂ spiked control sample.

To determine this range, the first step was to find the lowest L-Met concentration for which noninferiority compared to the control sample could be demonstrated. This was evaluated starting from the highest dose down to the lowest dose. The acceptance criteria to select this dose were based on a difference margin 6% by Mass Spectrometry (i.e. we looked for a deviation of no more than 6% of M192 oxidation from the reference, by mass spectrometry) or equivalent criteria in terms of oxidation peaks surface area for hydrophobic variants RP-HPLC.

Rather than measuring the methionine oxidation only directly by mass spectrometry, it was also estimated by RP-HPLC. It was found that the sum of RP-HPLC the oxidation peaks 1, 2 and 3 (see below) correlated well with the mass spectrometry measurements for M192 oxidation. Furthermore, the % area of peak 3 alone was found to be more than acceptable to correlate with mass spectrometry. The RP-HPLC method had the advantage of being faster and less variable at low oxidation values.

Results and Discussion

Hydrophobic Variants by RP-HPLC

RP-HPLC was used to look at purity.

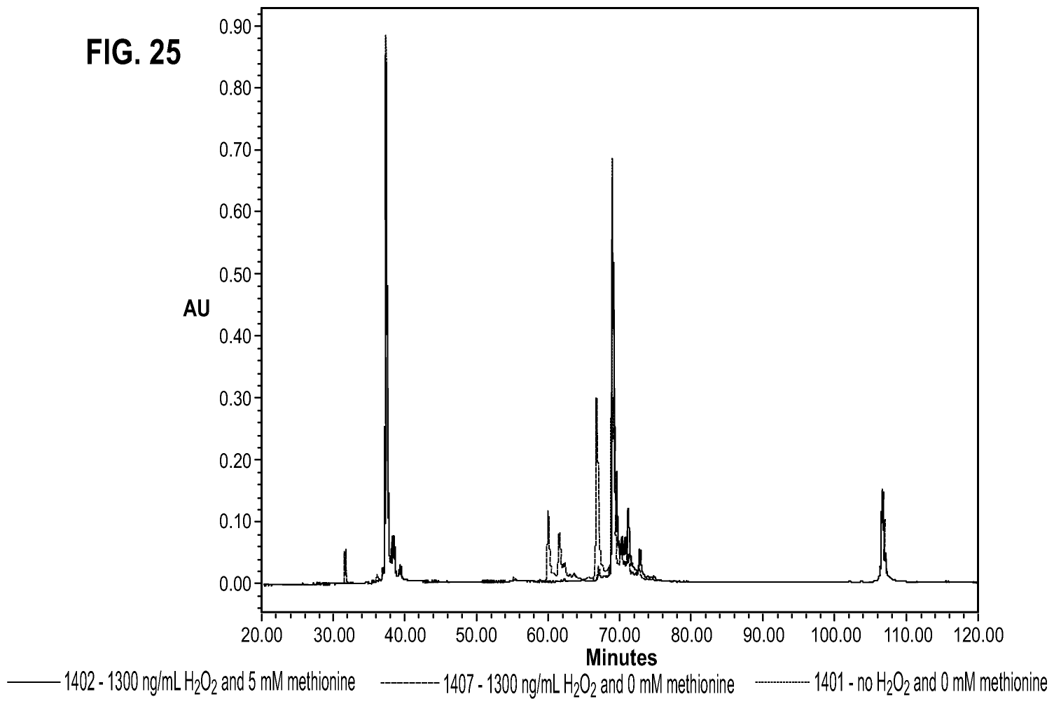

FIG. 25 shows hydrophobic variants HPLC 154 minutes chromatogram after 2 weeks 45° C. for samples 18COP1407 (0 mM L-Met+H₂O₂), 18COP1402 (5 mM L-Met+H₂O₂) and 18COP1401 (0 mM Met+no H₂O₂).

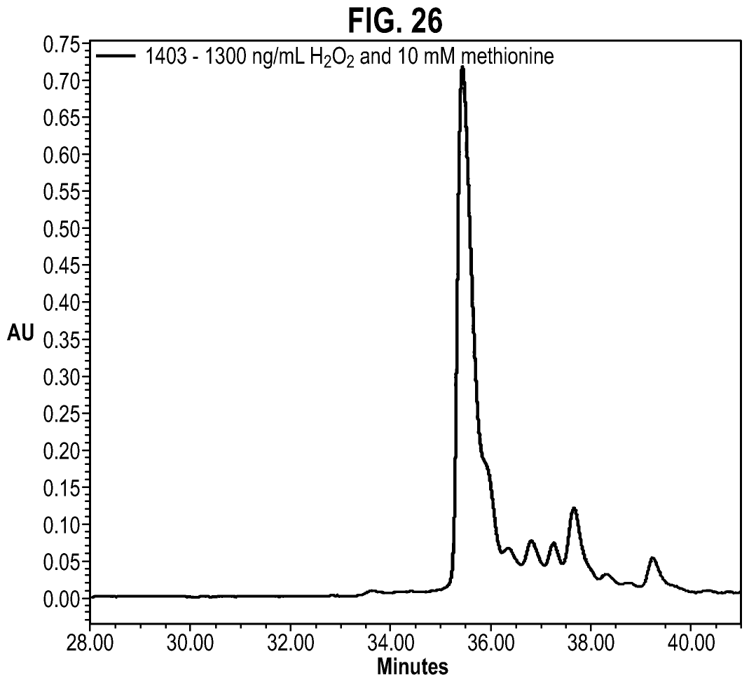

FIG. 26 shows hydrophobic variants HPLC minutes chromatogram after 2 weeks 45° C. for samples 18COP1403 (10 mM L-Met+H₂O₂).

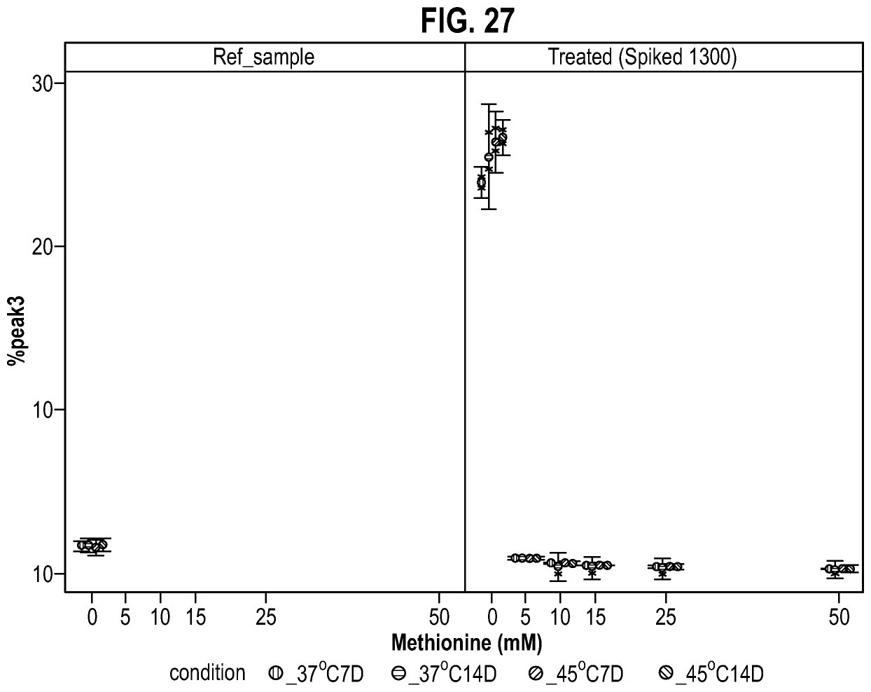

FIG. 27 shows hydrophobic variants RP-HPLC % peak3, in the left panel not oxidized samples without antioxidant; in the right panel oxidized samples with methionine at different concentrations.

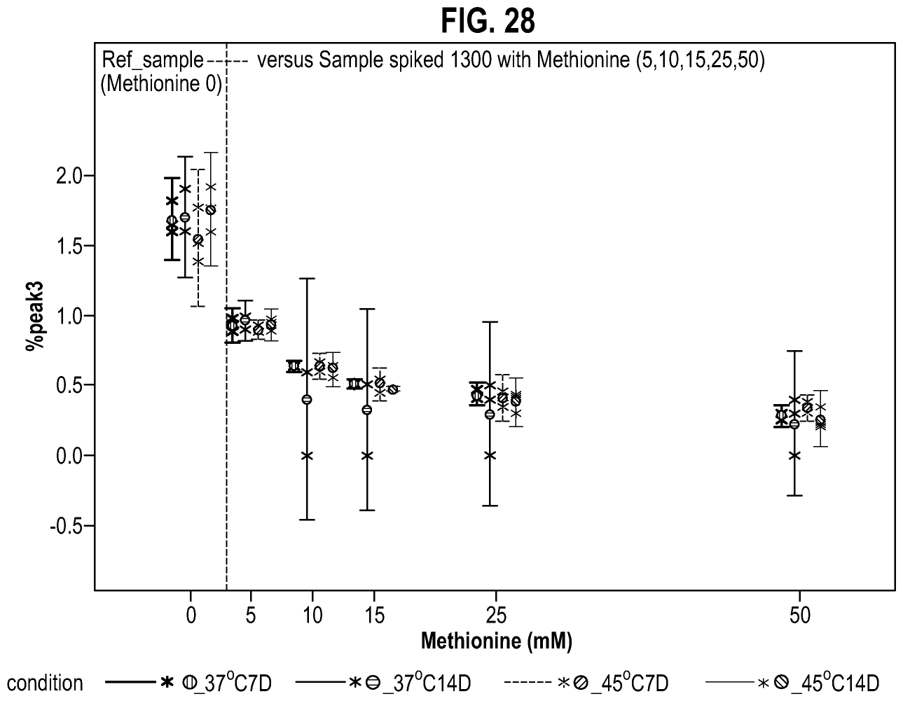

FIG. 28 shows hydrophobic variants RP-HPLC % peak3 oxidized samples with methionine at different concentrations.

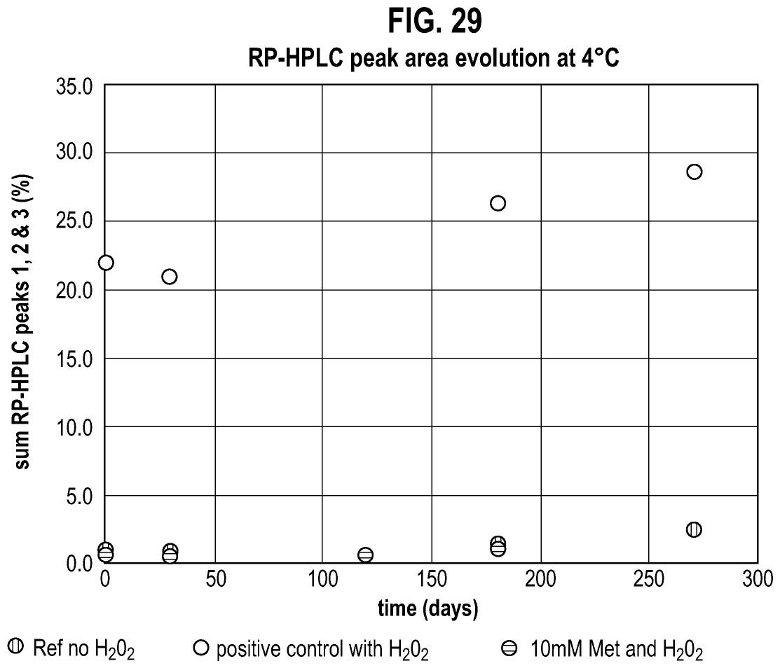

FIG. 29 shows the sum of area of peaks 1, 2 and 3 by RP-HPLC.

After 2 weeks at 45° C. no peaks were observed around 60-62 minutes for the sample containing 5 mM L-Met and H₂O₂ and for the reference sample containing no Methionine and no H₂O₂ (FIG. 25). After 67 minutes a slight oxidation peak was observed for both these samples. However, the peaks showed similar intensity. For the sample containing H₂O₂ but no methionine on the other hand, clear peaks were observed around 60, 62 and 67 minutes, named peaks 1, 2 and 3 respectively. Identical observations were made after 1 week 45° C. for the overlay with 10 mM of Methionine for which a chromatographic run focusing on protein D was performed.

No changes were observed in the profile of PE-PilA and UspA2 due to the presence of Methionine (FIG. 25). PE-PilA and UspA2 could be seen around 38 and 108 minutes respectively on the chromatogram. The small peak around 32 minutes for the sample containing H₂O₂ but no Methionine, was also observed during a PE-PilA analytical stress test exercise when PE-PilA was spiked with H₂O₂.

After 2 weeks at 45° C., for the sample containing H₂O₂ and 10 mM Methionine, no oxidation peaks were observed before the main protein D peak (FIG. 26), as was the case for the sample containing H₂O₂ and 5 mM Methionine and (FIG. 25). The overlay of the samples containing H₂O₂ and 5, 10 and 15 mM of Methionine after 1 week at 45° C. superimpose well and no meaningful oxidation peaks were observed before the main protein D peak for any of these samples (not shown).

The hydrophobic variants RP-HPLC % peak3 area is peak 3 area expressed as a percentage of the area of all the peaks together. % peak3 area showed a clear increase from around 2% for non-spiked reference samples (0 mM Met) up to around 27% for samples with no Methionine and spiked with 1300 ng of H₂O₂ per mL (see FIG. 27). For samples containing 5 mM of Methionine or more that were spiked with H₂O_{2,} no such increase in the hydrophobic variants RP-HPLC % peak3 area was observed. The evolution of the RP-HPLC % peak3 area between 0 and 5 mM L-Methionine was unknown, though it was noted that the increase of % peak3 had to have been sharp at some point since around 27% was observed for samples spiked with H₂O₂ containing no methionine.

Moreover, it was observed that the % peak3 area for samples with methionine and H₂O₂ was lower than for the reference sample containing no methionine and no spiked H₂O₂ (see FIG. 28). It was hypothesised this was due to some slight oxidation of the reference sample during the formulation, filling and freeze-drying processes while no methionine was present in the formulation to protect from this oxidation. Samples containing methionine (and spiked with H₂O₂) were protected from oxidation during this processing due to the presence of methionine. This could explain why a lower % peak3 area was observed for samples spiked with H₂O₂ and containing Methionine compared to the non-spiked no methionine reference sample.

Hereafter a summary of the statistical analysis is given that was performed on the Peak 3 area. Peak 3 was found more suitable for analysis than peak 2, as the observed signal for peak 2 was weak.

In samples spiked with 1300 ng H₂O₂/mL, Peak 3 was observed at Day 7 and 14, 37° C. or 45° C. For samples which contained at least 5 mM of Methionine results for Area Peak 3 reached the noninferiority criteria, since the upper limit of the 2-sided standardized asymptotic 90% CI for the group difference [treated minus control] was below 387000 and 260000 respectively [limit for noninferiority]). This corresponded to an acceptable difference of 9% and 6% respectively measured by Mass Spectrometry.

The non-inferiority criteria were not met for samples spiked with 1300 ng H₂O₂/mL in the absence of methionine.

Methionine Oxidation by Liquid Chromatography Coupled Mass Spectrometry

Protein D

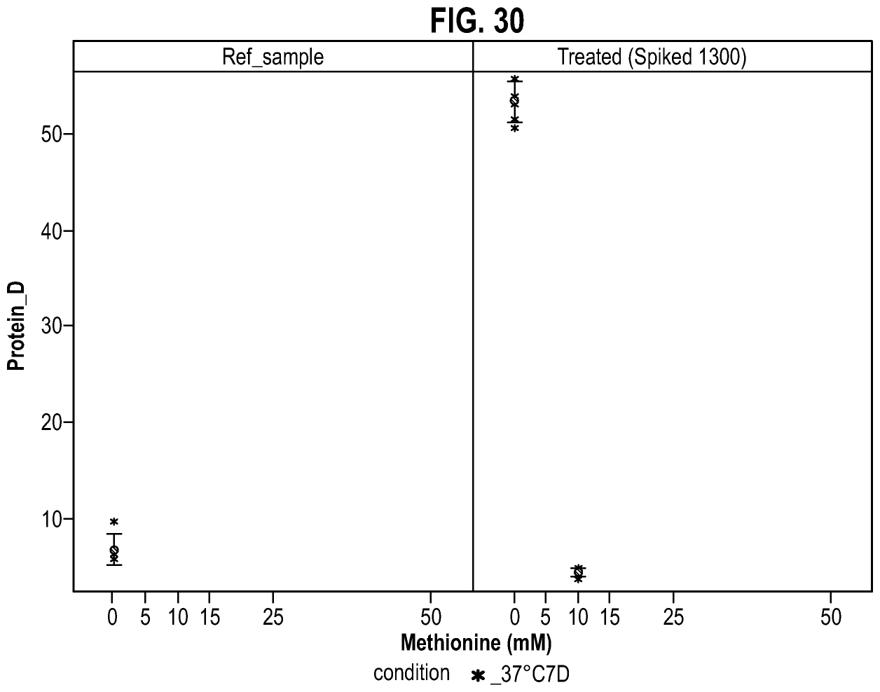

FIG. 30 shows liquid chromatography coupled mass spectrometry for protein D M192 oxidation in % after 1 month at 37° C. The left panel contains samples not spiked with H₂O₂, in the right panel samples received 1300 ng of H₂O₂ per mL before freeze drying. The error bars indicate the 95% confidence intervals.

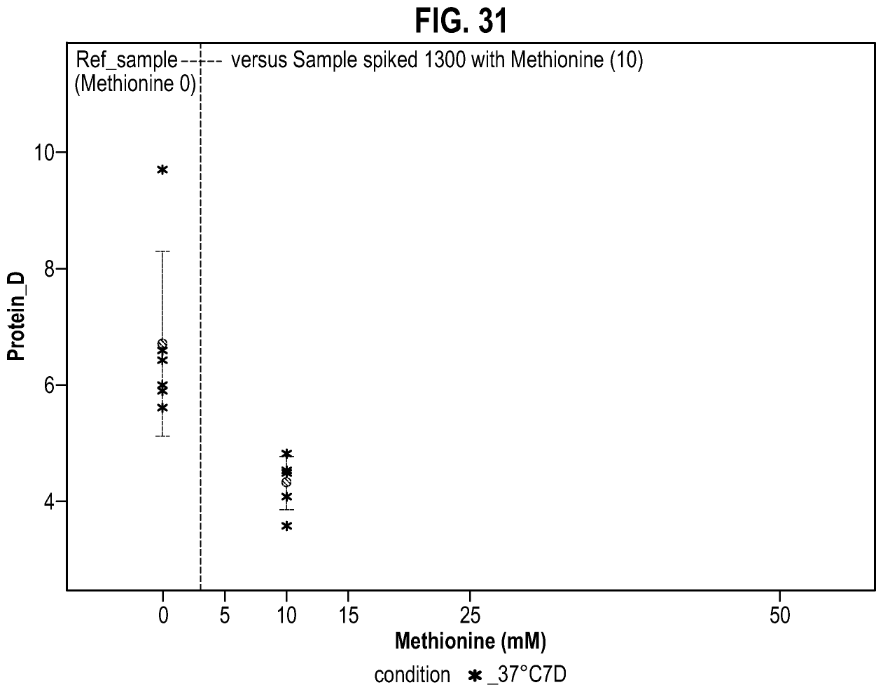

FIG. 31 shows liquid chromatography coupled mass spectrometry for protein D M192 oxidation in % after 1 month at 37° C. The left panel contains samples not spiked with H₂O₂, in the right panel samples received 1300 ng of H₂O₂ per mL before freeze drying and contain 10 mM of Methionine. The error bars indicate the 95% confidence intervals.

Mass spectrometry data for protein D Methionine 192 (M192) are depicted in FIG. 30. The sample that was not spiked with H₂O₂ and contained no Methionine showed very limited levels of M192 oxidation, whereas the sample spiked with H₂O₂ and containing no Methionine, clearly showed a high level of M192 oxidation—around 50%, and did not meet the statistical noninferiority criterion. The sample containing 10 mM of L-Met and spiked with H₂O₂ had an oxidation level lower or equal to the non-spiked reference. This sample met the statistical non-inferiority criterion, since the upper limit of the 2-sided standardized asymptotic 90% CI for the group difference [treated minus control] was below 6% [limit for non-inferiority]. As for the hydrophobic variants RP-HPLC, the oxidation seemed slightly less for samples containing methionine compared to the non-spiked non-methionine containing samples (FIG. 31). A possible explanation for this observation is given above in the discussion of the RP-HPLC results.

PE-PilA

For PE-PilA M215 oxidation, the levels of oxidation observed after 30 days at 37° C. were in the same range for all the tested samples (data not shown). No difference between the non H₂O₂ spiked reference and the H₂O₂ spiked sample containing 10 mM Methionine could be found.

UspA2

For UspA2 M530 oxidation, the sample that was not spiked with H₂O₂ and contained no Methionine showed very limited levels of M530 oxidation (around 2%). The sample spiked with H₂O₂ and containing no Methionine, clearly showed a higher level of M530 oxidation; around 8% and did meet the statistical non-inferiority criterion. The sample containing 10 mM of L-Met and spiked with H₂O₂ had an oxidation level lower than the non-spiked reference (data not shown).

Molar Considerations

Since oxidation is a chemical reaction it is interesting to express the quantities of oxidants and antioxidants in moles to get an idea of the molar ratios.

Molar wise the quantities of reactant and reagent are the following;

| Quantity | Molar Quantity |
|---|---|
| 1300 ng/mL H₂O₂ spiked | 0.038 mM |
| Protein D concentration (25 μg/mL in drug product, | 0.0006 mM |
| 40 kDa per Protein D molecule) | |

It can be seen there is a 63-fold surplus of H₂O₂ molecules compared to Protein D. However, if 10 mM of Methionine is added to the drug product, there are 263 molecules of Methionine for each molecule of H₂O₂ spiked at 1300 ng/mL. Therefore, the addition of methionine greatly decreases the chances of H₂O₂ reacting with protein D.

Conclusions

We showed that oxidation of protein D was observed for an equivalent manufacturing process executed at 0.1 ppm v/v or 1 ppm v/v H₂O₂ exposure in the gas phase. We demonstrated the addition of an antioxidant, specifically L-Methionine or cysteine, could prevent such oxidation.

The following points were taken into consideration when deciding on the Methionine concentration to be added to the drug product;
[Met] should protect for a 1 ppm v/v H₂O₂ process in the isolator to ensure manufacturing flexibility
10 mM of Met gives a sufficient safety margin and a data point at a lower concentration (5 mM) for which the RP-HPLC peak 3 area remains below the non-oxidized reference (no H₂O₂ spiked)
10 mM of Methionine has demonstrated good protection from oxidation based on mass spectrometry results for sensitive methionines on the 3 antigens present in the composition containing Protein D, PEPilA and UspA2.
For these reasons a concentration of 10 mM L-Met was selected in this example for this vaccine.

### Example 5

### Antioxidants for Live Vector Vaccine

A ChAd155-RSV adenovirus vector was assessed for potential oxidation by residual VHP used for sanitization of commercial filling/transfer lines.

The ChAd155-RSV vector used herein contains RSV transgenes encoding the F, N, M2 structural proteins from Respiratory Syncytial Virus. The transgenes were inserted in the adenoviral vector after deletion of the ChAd155 E1 and most of the E4 regions. Furthermore, to improve the productivity of the ChAd155 vector in human packaging cell line expressing the Ad5 E1 region, the native Chimpanzee E4 region is substituted with Ad5 E4orf6.

The live vector vaccine was spiked with H₂O₂ at 0, 150 and 1300 ng/mL H₂O₂, representing conditions of 0 ppm, 0.1 ppm and 1 ppm VHP in commercial facilities.

Experiments were performed with and without methionine and at difference doses of methionine. Vaccine doses were then filled and freeze dried and accelerated stability studies were performed.

The following methods were used to assess the impact of H₂O₂/antioxidant on the live vector vaccine.

Viral infectivity was measured by FACS analysis. Viral particle content was measured by HPLC. Viral DNA content was measured by qPCR (quantitative PCR). Viral capsid integrity was measured by DNA release using a Picogreen assay. Details are given below.

TABLE 6
Picogreen assay experimental conditions

| | |
|---|---|
| General | Quant-iT ™ PicoGreen ® dsDNA reagent was an ultra-sensitive fluorescent |
| information | nucleic acid stain for quantitating double-stranded DNA (dsDNA) in solution. |
| | The test was used to assess viral integrity as detection of DNA in samples |
| | is linked to capsid lysis. |
| Equipment | Varioskan Flash system Thermo scientific -Tag 77194 & SAP number 224673 |
| Consumables | Quant-IT Picogreen dsDNA Assay kit (Invitrogen, ref. P 7589) |
| | Multi-96 well plates 100 μl UV-bottom transparent (Corning, NY 14831-ref. 3679) |
| Theoretical | From 25 pg/mL to 1,000 ng/mL |
| range | |

PicoGreen assay was performed on fresh and degraded controls of DS that are necessary to normalize the standardized values obtained for samples. The standardized values were obtained from the standard curve of the DNA reagent kit. Calculation of normalization was then performed from the standardized value of the fresh control (considered as 0% of the DNA release in the matrix) and the degraded control (considered as 100% of the DNA release in the matrix), by relating value of samples to the standard straight line calculated between both controls. The degraded control was obtained by subjecting the DS diluted to the formulation concentration, to 60° C. for 30 min.

TABLE 7
Infectivity by FACS experimental conditions

| | |
|---|---|
| General | “Infectivity” refers to the ability of a vector to enter a susceptible host. |
| information | The infectivity by FACS (Fluorescence-activated cell sorting) assay was an |
| | adenovirus-specific quantification assay of the infected cells through |
| | transgene expression. |
| | HEK293 cells were cultured, then infected with adenovirus particles and |
| | incubated for 21-24 hours at 37° C. Cells were then stained with anti-M |
| | antibodies. FACS was then used to detect protein M expression in the |
| | infected cells. The quantification was based on the number of positive cells. |
| Equipment | FACS BD LSR II Tag 224957 or Tag 226332 or FACS BD Fortessa Tag 240524 |
| Consumables | Multi-96 well plates |
| Theoretical | 1E7 to 1E11 infectious particles/mL or per dose |
| range | |

Results for HPLC and qPCR showed no significant impact of spiking with H₂O₂. This showed that oxidation did not completely alter the integrity of the virus particles or the DNA, thus particle-content and whole DNA remained stable after H₂O₂ spiking.

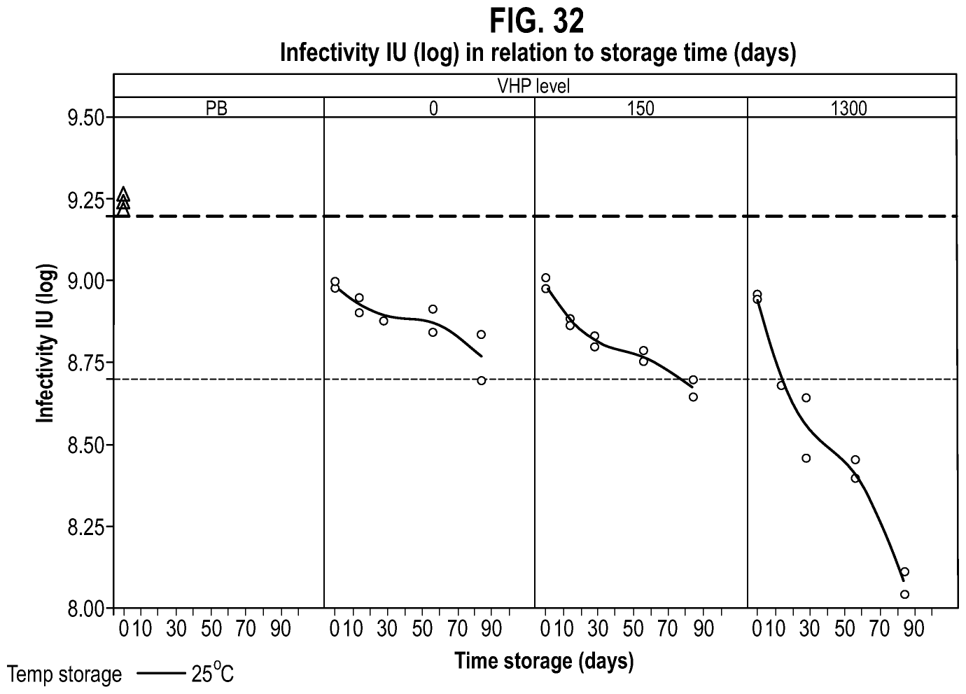

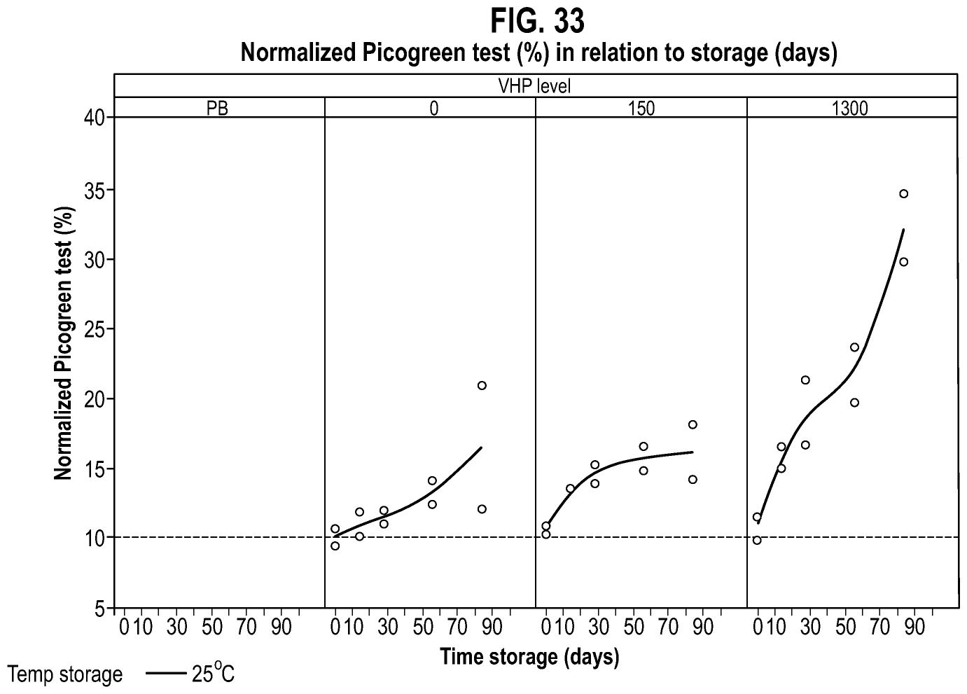

However, infectivity by FACS analysis and DNA release by Picogreen assay were affected and are shown in FIGS. 32 and 33. These tests (average±SD, N=2) showed that conditions representing 0.1 and 1 ppm residual VHP significantly impacted both CQAs after one month at 25° C. (1M25° C.). This showed that oxidation both altered capsid integrity and decreased the virus ability to infect cells.

A dose ranging study was performed using methionine concentrations of between 0 and 25 mM, 1M25° C.

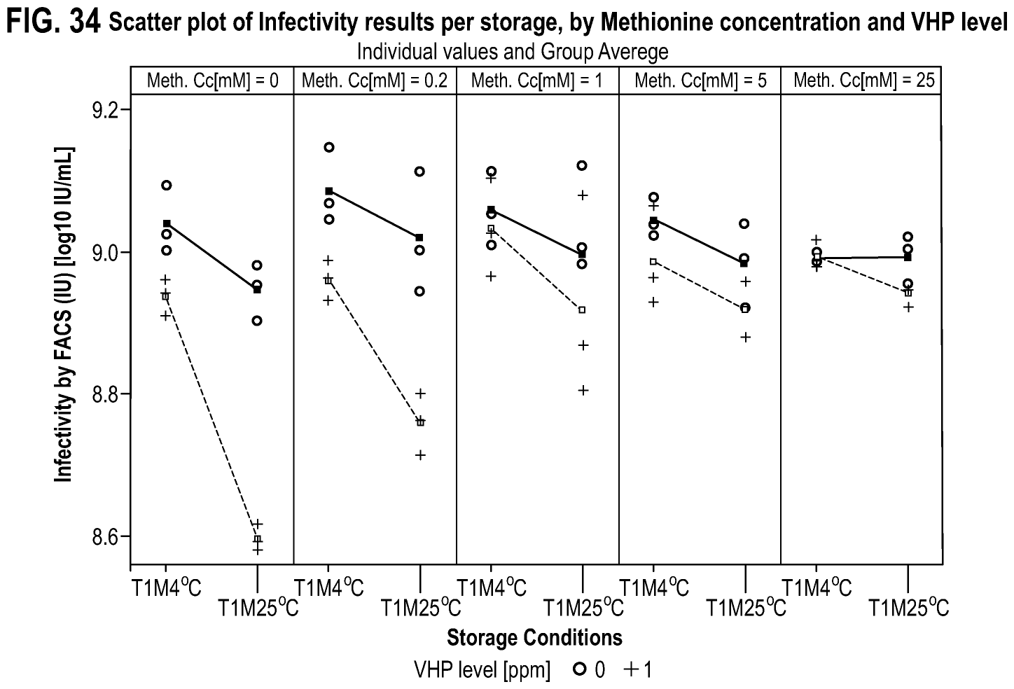

For the dose ranging study, infectivity by FACS is shown in FIG. 34. Results were consistent with the previous study (0.4 log loss between T0 and T1M25° C. with 1 ppm VHP). In the absence of VHP, the difference in infectivity between T1M25° C. and T1M4° C. was relatively stable across methionine concentrations. In the presence of VHP, increasing the methionine concentration significantly improved the difference in infectivity between T1M25° C. and T1M4° C., with a plateau which seemed to be reached around 5 mM methionine.

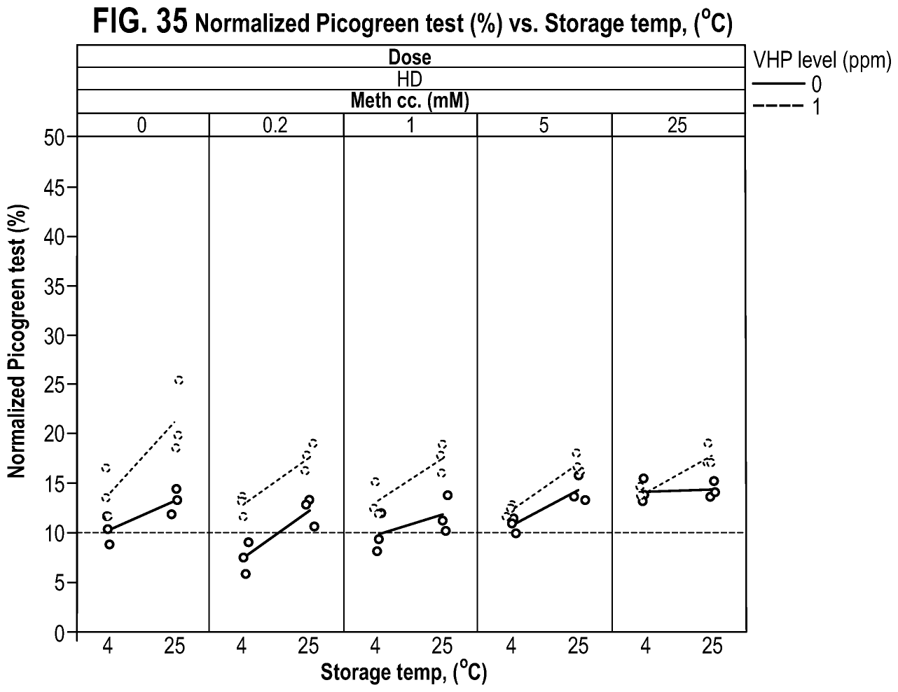

Capsid integrity by Picogreen is shown in FIG. 35. Picogreen % is the ratio between the measured fluorescence of the sample and a degraded control. The degraded control was a sample of the composition diluted to the concentration of the formulation, subjected to 30 minutes at 60° C.

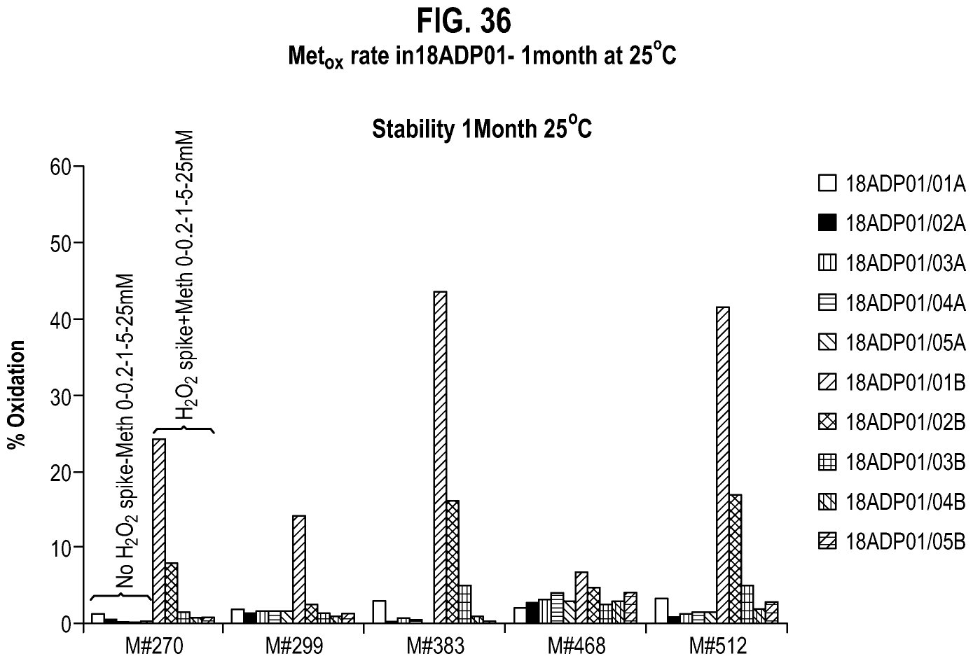

ChAd155 Hexon Methionine Oxidation was measured by LC-MS and results for five of the methionines (Met270, 299, 383, 468 and 512) are shown in FIG. 36. The hexon protein is the adenovirus major coat protein and has large numbers of methionines. Met270, 299, 383, 468 and 512 were selected based on their location, sensitivity and oxidation rate. The ChAd155 hexon Protein II major capsid protein sequence is given in SEQ ID NO: 21.

Results showed that 5 mM methionine or greater prevented the effect of 1 ppm VHP on the live vector vaccine and that methionine also protected the vaccine from the effect of lyophilisation even in the absence of H₂O₂. In FIG. 36 the first five bars for each methionine show increasing amounts of methionine (starting with zero) added in the absence of H₂O₂. The second five bars show increasing methionine in the presence of equivalent of 1 ppm VHP. A protective effect of methionine can also be clearly seen when the average for the five methionines shown in FIG. 36 is calculated.

Thus 5 mM methionine and above was established as able to control the impact of VHP on CQAs after T1M25 and on MetOx ratios.

This example shows that Methionine addition is again an effective solution to counteract the effects of oxidation linked to process stresses (freeze-drying and H₂O₂ exposure), this time on a live virus vaccine.

SEQUENCES
An RSV PreF sequence
SEQ ID NO: 1
MELLILKTNAITAILAAVTLCFASSQNITEEFYQSTCSAVSKGYLSALRTGWYTSVITIELSNIKENKCNGTDAKVKLIKQELD
KYKSAVTELQLLMQSTPATNNKFLGFLQGVGSAIASGIAVSKVLHLEGEVNKIKSALLSTNKAVVSLSNGVSVLTSKVLDL
KNYIDKQLLPIVNKQSCSISNIETVIEFQQKNNRLLEITREFSVNAGVTTPVSTYMLTNSELLSLINDMPITNDQKKLMSNN
VQIVRQQSYSIMSIIKEEVLAYVVQLPLYGVIDTPCWKLHTSPLCTTNTKEGSNICLTRTDRGWYCDNAGSVSFFPLAETC
KVQSNRVFCDTMNSLTLPSEVNLCNIDIFNPKYDCKIMTSKTDVSSSVITSLGAIVSCYGKTKCTASNKNRGIIKTFSNGCD
YVSNKGVDTVSVGNTLYYVNKQEGKSLYVKGEPIINFYDPLVFPSDEFDASISQVNEKINGSLAFIRKSDEKLHNVEDKIEEI
LSKIYHIENEIARIKKLIGEA
An RSV PreF sequence which is part of SEQ ID NO: 1
SEQ ID NO: 2
SSQNITEEFYQSTCSAVSKGYLSALRTGWYTSVITIELSNIKENKCNGTDAKVKLIKQELDKYKSAVTELQLLMQSTPATNN
KFLGFLQGVGSAIASGIAVSKVLHLEGEVNKIKSALLSTNKAVVSLSNGVSVLTSKVLDLKNYIDKQLLPIVNKQSCSISNIET
VIEFQQKNNRLLEITREFSVNAGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPLAETCKVQSNRVFCDTMNSLTLPSEV
NLCNIDIFNPKYDCKIMTSKTDVSSSVITSLGAIVSCYGKTKCTASNKNRGIIKTFSNGCDYVSNKGVDTVSVGNTLYYVNK
QEGKSLYVKGEPIINFYDPLVFPSDEFDASISQVNEKINGSLAFIRKSDEKLHNVEDKIEEILSKIYHIENEIARIKKLIGEA
A further RSV PreF sequence
SEQ ID NO: 3
MELLILKANAITTILTAVTFCFASGQNITEEFYQSTCSAVSKGYLSALRTGWYTSVITIELSNIKENKCNGTDAKVKLIKQELD
KYKNAVTELQLLMQSTPATNNRARRELPRFMNYTLNNAKKTNVTLSKKRKRRFLGFLLGVGSAIASGVAVCKVLHLEGE
VNKIKSALLSTNKAVVSLSNGVSVLTFKVLDLKNYIDKQLLPILNKQSCSISNIETVIEFQQKNNRLLEITREFSVNAGVTTPV
STYMLTNSELLSLINDMPITNDQKKLMSNNVQIVRQQSYSIMCIIKEEVLAYVVQLPLYGVIDTPCWKLHTSPLCTTNTKE
GSNICLTRTDRGWYCDNAGSVSFFPQAETCKVQSNRVFCDTMNSLTLPSEVNLCNVDIFNPKYDCKIMTSKTDVSSSVI
TSLGAIVSCYGKTKCTASNKNRGIIKTFSNGCDYVSNKGVDTVSVGNTLYYVNKQEGKSLYVKGEPIINFYDPLVFPSDEF
DASISQVNEKINQSLAFIRKSDELLSAIGGYIPEAPRDGQAYVRKDGEWVLLSTFL
A further RSV PreF sequence
SEQ ID NO: 4
MELLILKTNAITAILAAVTLCFASSQNITEEFYQSTCSAVSKGYLSALRTGWYTSVITIELSNIKENKCNGTDAKVKLIKQELD
KYKSAVTELQLLMQSTPATNNKFLGFLQGVGSAIASGIAVSKVLHLEGEVNKIKSALLSTNKAVVSLSNGVSVLTSKVLDL
KNYIDKQLLPIVNKQSCSISNIETVIEFQQKNNRLLEITREFSVNAGVTTPVSTYMLTNSELLSLINDMPITNDQKKLMSN N
VQIVRQQSYSIMSIIKEEVLAYVVQLPLYGVIDTPCWKLHTSPLCTTNTKEGSNICLTRTDRGWYCDNAGSVSFFPLAETC
KVQSNRVFCDTMNSLTLPSEVNLCNIDIFNPKYDCKIMTSKTDVSSSVITSLGAIVSCYGKTKCTASNKNRGIIKTFSNGCD
YVSNKGVDTVSVGNTLYYVNKQEGKSLYVKGEPIINFYDPLVFPSDEFDASISQVNEKINGTLAFIRKSDEKLHNVEDKIEE
ILSKIYHIENEIARIKKLIGEA
A further RSV PreF sequence
SEQ ID NO: 5
MELLILKTNAITAILAAVTLCFASSQNITEEFYQSTCSAVSKGYLSALRTGWYTSVITIELSNIKENKCNGTDAKVKLIKQELD
KYKSAVTELQLLMQSTPATNNKFLGFLLGVGSAIASGIAVSKVLHLEGEVNKIKSALLSTNKAVVSLSNGVSVLTSKVLDLK
NYIDKQLLPIVNKQSCSISNIETVIEFQQKNNRLLEITREFSVNAGVTTPVSTYMLTNSELLSLINDMPITNDQKKLMSNNV
QIVRQQSYSIMSIIKEEVLAYVVQLPLYGVIDTPCWKLHTSPLCTTNTKEGSNICLTRTDRGWYCDNAGSVSFFPLAETCK
VQSNRVFCDTMNSLTLPSEVNLCNIDIFNPKYDCKIMTSKTDVSSSVITSLGAIVSCYGKTKCTASNKNRGIIKTFSNGCDY
VSNKGVDTVSVGNTLYYVNKQEGKSLYVKGEPIINFYDPLVFPSDEFDASISQVNEKINQSLAFIRKSDEKLHNVEDKIEEIL
SKIYHIENEIARIKKLIGEA
A coiled-coil (isoleucine zipper) sequence
SEQ ID NO: 6
EDKIEEILSKIYHIENEIARIKKLIGEA
F1 chain of mature polypeptide produced from the precursor sequence shown in SEQ ID
NO: 3
SEQ ID NO: 7
FLGFLLGVGSAIASGVAVCKVLHLEGEVNKIKSALLSTNKAVVSLSNGVSVLTFKVLDLKNYIDKQLLPILNKQSCSISNIETV
IEFQQKNNRLLEITREFSVNAGVTTPVSTYMLTNSELLSLINDMPITNDQKKLMSNNVQIVRQQSYSIMCIIKEEVLAYVV
QLPLYGVIDTPCWKLHTSPLCTTNTKEGSNICLTRTDRGWYCDNAGSVSFFPQAETCKVQSNRVFCDTMNSLTLPSEVN
LCNVDIFNPKYDCKIMTSKTDVSSSVITSLGAIVSCYGKTKCTASNKNRGIIKTFSNGCDYVSNKGVDTVSVGNTLYYVNK
QEGKSLYVKGEPIINFYDPLVFPSDEFDASISQVNEKINQSLAFIRKSDELLSAIGGYIPEAPRDGQAYVRKDGEWVLLSTFL
F2 chain of mature polypeptide produced from the precursor sequence shown in SEQ ID
NO: 3
SEQ ID NO: 8
QNITEEFYQSTCSAVSKGYLSALRTGWYTSVITIELSNIKENKCNGTDAKVKLIKQELDKYKNAVTELQLLMQSTPATNNR
ARR
Substance P (model peptide used in the Examples)
SEQ ID NO: 9
RPKPQQFFGLM
Protein D (364 amino acids)
SEQ ID NO: 10
MetLysLeuLysThrLeuAlaLeuSerLeuLeuAlaAlaGlyValLeuAlaGly
CysSerSerHisSerSerAsnMetAlaAsnThrGlnMetLysSerAspLysIle
IleIleAlaHisArgGlyAlaSerGlyTyrLeuProGluHisThrLeuGluSerLysAla
LeuAlaPheAlaGlnGlnAlaAspTyrLeuGluGlnAspLeuAlaMetThrLysAspGly
ArgLeuValValIleHisAspHisPheLeuAspGlyLeuThrAspValAlaLysLysPhe
ProHisArgHisArgLysAspGlyArgTyrTyrValIleAspPheThrLeuLysGluIle
GlnSerLeuGluMetThrGluAsnPheGluThrLysAspGlyLysGlnAlaGlnValTyr
ProAsnArgPheProLeuTrpLysSerHisPheArgIleHisThrPheGluAspGluIle
GluPheIleGlnGlyLeuGluLysSerThrGlyLysLysValGlyIleTyrProGluIle
LysAlaProTrpPheHisHisGlnAsnGlyLysAspIleAlaAlaGluThrLeuLysVal
LeuLysLysTyrGlyTyrAspLysLysThrAspMetValTyrLeuGlnThrPheAspPhe
AsnGluLeuLysArgIleLysThrGluLeuLeuProGlnMetGlyMetAspLeuLysLeu
ValGlnLeuIleAlaTyrThrAspTrpLysGluThrGlnGluLysAspProLysGlyTyr
TrpValAsnTyrAsnTyrAspTrpMetPheLysProGlyAlaMetAlaGluValValLys
TyrAlaAspGlyValGlyProGlyTrpTyrMetLeuValAsnLysGluGluSerLysPro
AspAsnIleValTyrThrProLeuValLysGluLeuAlaGlnTyrAsnValGluValHis
ProTyrThrValArgLysAspAlaLeuProGluPhePheThrAspValAsnGlnMetTyr
AspAlaLeuLeuAsnLysSerGlyAlaThrGlyValPheThrAspPheProAspThrGly
ValGluPheLeuLysGlyIleLys
Protein D fragment with MDP tripeptide from NS1 (348 amino acids)
SEQ ID NO: 11
MetAspProSerSerHisSerSerAsnMetAlaAsnThrGlnMetLysSerAspLysIle
IleIleAlaHisArgGlyAlaSerGlyTyrLeuProGluHisThrLeuGluSerLysAla
LeuAlaPheAlaGlnGlnAlaAspTyrLeuGluGlnAspLeuAlaMetThrLysAspGly
ArgLeuValValIleHisAspHisPheLeuAspGlyLeuThrAspValAlaLysLysPhe
ProHisArgHisArgLysAspGlyArgTyrTyrValIleAspPheThrLeuLysGluIle
GlnSerLeuGluMetThrGluAsnPheGluThrLysAspGlyLysGlnAlaGlnValTyr
ProAsnArgPheProLeuTrpLysSerHisPheArgIleHisThrPheGluAspGluIle
GluPheIleGlnGlyLeuGluLysSerThrGlyLysLysValGlyIleTyrProGluIle
LysAlaProTrpPheHisHisGlnAsnGlyLysAspIleAlaAlaGluThrLeuLysVal
LeuLysLysTyrGlyTyrAspLysLysThrAspMetValTyrLeuGlnThrPheAspPhe
AsnGluLeuLysArgIleLysThrGluLeuLeuProGlnMetGlyMetAspLeuLysLeu
ValGlnLeuIleAlaTyrThrAspTrpLysGluThrGlnGluLysAspProLysGlyTyr
TrpValAsnTyrAsnTyrAspTrpMetPheLysProGlyAlaMetAlaGluValValLys
TyrAlaAspGlyValGlyProGlyTrpTyrMetLeuValAsnLysGluGluSerLysPro
AspAsnIleValTyrThrProLeuValLysGluLeuAlaGlnTyrAsnValGluValHis
ProTyrThrValArgLysAspAlaLeuProGluPhePheThrAspValAsnGlnMetTyr
AspAlaLeuLeuAsnLysSerGlyAlaThrGlyValPheThrAspPheProAspThrGly
ValGluPheLeuLysGlyIleLys
Start of the protein D fragment described in EP0594610
SEQ ID NO: 12
SSHSSNMANT
Protein E from H. influenzae
SEQ ID NO: 13
MKKIILTLSLGLLTACSAQIQKAEQNDVKLAPPTDVRSGYIRLVKNVNYYIDSESIWVDNQEPQIVHFDAVVNLDKGLYVY
PEPKRYARSVRQYKILNCANYHLTQVRTDFYDEFWGQGLRAAPKKQKKHTLSLTPDTTLYNAAQIICANYGEAFSVDKK
Amino acids 20-160 of Protein E from H. influenzae
SEQ ID NO: 14
IQKAEQNDVKLAPPTDVRSGYIRLVKNVNYYIDSESIWVDNQEPQIVHFDAVVNLDKGLYVYPEPKRYARSVRQYKILNC
ANYHLTQVRTDFYDEFWGQGLRAAPKKQKKHTLSLTPDTTLYNAAQIICANYGEAFSVDKK
PilA from H. influenzae
SEQ ID NO: 15
MKLTTQQTLKKGFTLIELMIVIAIIAILATIAIPSYQNYTKKAAVSELLQASAPYKADVELCVYSTNETTNCTGGKNGIAADI
TTAKGYVKSVTTSNGAITVKGDGTLANMEYILQATGNAATGVTWTTTCKGTDASLFPANFCGSVTQ
Amino acids 40-149 of PilA from H. influenzae strain 86-028NP
SEQ ID NO: 16
TKKAAVSELLQASAPYKADVELCVYSTNETTNCTGGKNGIAADITTAKGYVKSVTTSNGAITVKGDGTLANMEYILQATG
NAATGVTWTTTCKGTDASLFPANFCGSVTQ
SEQ ID NO: 17
MKYLLPTAAAGLLLLAAQPAMAIQKAEQNDVKLAPPTDVRSGYIRLVKNVNYYIDSESIWVDNQEPQIVHFDAVVNLD
KGLYVYPEPKRYARSVRQYKILNCANYHLTQVRTDFYDEFWGQGLRAAPKKQKKHTLSLTPDTTLYNAAQIICANYGEA
FSVDKKGGTKKAAVSELLQASAPYKADVELCVYSTNETTNCTGGKNGIAADITTAKGYVKSVTTSNGAITVKGDGTLAN
MEYILQATGNAATGVTWTTTCKGTDASLFPANFCGSVTQ
PE-PilA fusion protein without signal peptide
SEQ ID NO: 18
IQKAEQNDVKLAPPTDVRSGYIRLVKNVNYYIDSESIWVDNQEPQIVHFDAVVNLDKGLYVYPEPKRYARSVRQYKILNC
ANYHLTQVRTDFYDEFWGQGLRAAPKKQKKHTLSLTPDTTLYNAAQIICANYGEAFSVDKKGGTKKAAVSELLQASAPY
KADVELCVYSTNETTNCTGGKNGIAADITTAKGYVKSVTTSNGAITVKGDGTLANMEYILQATGNAATGVTWTTTCKG
TDASLFPANFCGSVTQ
UspA2 A2 from Moraxella catarrhalis (from ATCC 25238)
SEQ ID NO: 19
MKTMKLLPLKIAVTSAMIIGLGAASTANAQAKNDITLEDLPYLIKKIDQNELEADIGDITALEKYLALSQYGNILALEELNKA
LEELDEDVGWNQNDIANLEDDVETLTKNQNALAEQGEAIKEDLQGLADFVEGQEGKILQNETSIKKNTQRNLVNGFEIE
KNKDAIAKNNESIEDLYDFGHEVAESIGEIHAHNEAQNETLKGLITNSIENTNNITKNKADIQALENNVVEELFNLSGRLID
QKADIDNNINNIYELAQQQDQHSSDIKTLKKNVEEGLLELSGHLIDQKTDIAQNQANIQDLATYNELQDQYAQKQTEAI
DALNKASSENTQNIEDLAAYNELQDAYAKQQTEAIDALNKASSENTQNIEDLAAYNELQDAYAKQQTEAIDALNKASSE
NTQNIAKNQADIANNINNIYELAQQQDQHSSDIKTLAKASAANTDRIAKNKADADASFETLTKNQNTLIEKDKEHDKLIT
ANKTAIDANKASADTKFAATADAITKNGNAITKNAKSITDLGTKVDGFDSRVTALDTKVNAFDGRITALDSKVENGMAA
QAALSGLFQPYSVGKFNATAALGGYGSKSAVAIGAGYRVNPNLAFKAGAAINTSGNKKGSYNIGVNYEF
Immunogenic fragment of UspA2 (31-564)
SEQ ID NO: 20
MAKNDITLEDLPYLIKKIDQNELEADIGDITALEKYLALSQYGNILALEELNKALEELDEDVGWNQNDIANLEDDVETLTK
NQNALAEQGEAIKEDLQGLADFVEGQEGKILQNETSIKKNTQRNLVNGFEIEKNKDAIAKNNESIEDLYDFGHEVAESIG
EIHAHNEAQNETLKGLITNSIENTNNITKNKADIQALENNVVEELFNLSGRLIDQKADIDNNINNIYELAQQQDQHSSDIK
TLKKNVEEGLLELSGHLIDQKTDIAQNQANIQDLATYNELQDQYAQKQTEAIDALNKASSENTQNIEDLAAYNELQDAY
AKQQTEAIDALNKASSENTQNIEDLAAYNELQDAYAKQQTEAIDALNKASSENTQNIAKNQADIANNINNIYELAQQQ
DQHSSDIKTLAKASAANTDRIAKNKADADASFETLTKNQNTLIEKDKEHDKLITANKTAIDANKASADTKFAATADAITK
NGNAITKNAKSITDLGTKVDGFDSRVTALDTKVNAFDGRITALDSKVENGMAAQAAHH
ChAd155 hexon Protein II major capsid protein
SEQ ID NO: 21
MATPSMMPQWSYMHISGQDASEYLSPGLVQFARATESYFSLSNKFRNPTVAPTHDVTTDRSQRLTLRFIP
VDREDTAYSYKARFTLAVGDNRVLDMASTYFDIRGVLDRGPTFKPYSGTAYNSLAPKGAPNSCEWEQEET
QAVEEAAEEEEEDADGQAEEEQAATKKTHVYAQAPLSGEKISKDGLQIGTDATATEQKPIYADPTFQPEP
QIGESQWNEADATVAGGRVLKKSTPMKPCYGSYARPTNANGGQGVLTANAQGQLESQVEMQFFSTSENAR
NEANNIQPKLVLYSEDVHMETPDTHLSYKPAKSDDNSKIMLGQQSMPNRPNYIGFRDNFIGLMYYNSTGN
MGVLAGQASQLNAVVDLQDRNTELSYQLLLDSMGDRTRYFSMWNQAVDSYDPDVRIIENHGTEDELPNYC
FPLGGIGVTDTYQAVKTNNGNNGGQVTWTKDETFADRNEIGVGNNFAMEINLSANLWRNFLYSNVALYLP
DKLKYNPSNVDISDNPNTYDYMNKRVVAPGLVDCYINLGARWSLDYMDNVNPFNHHRNAGLRYRSMLLGN
GRYVPFHIQVPQKFFAIKNLLLLPGSYTYEWNFRKDVNMVLQSSLGNDLRVDGASIKFESICLYATFFPM
AHNTASTLEAMLRNDTNDQSFNDYLSAANMLYPIPANATNVPISIPSRNWAAFRGWAFTRLKTKETPSLG
SGFDPYYTYSGSIPYLDGTFYLNHTFKKVSVTFDSSVSWPGNDRLLTPNEFEIKRSVDGEGYNVAQCNMT
KDWFLVQMLANYNIGYQGFYIPESYKDRMYSFFRNFQPMSRQVVDQTKYKDYQEVGIIHQHNNSGFVGYL
APTMREGQAYPANFPYPLIGKTAVDSITQKKFLCDRTLWRIPFSSNFMSMGALSDLGQNLLYANSAHALD
MTFEVDPMDEPTLLYVLFEVFDVVRVHQPHRGVIETVYLRTPFSAGNATT

1. A method of manufacturing a biological medicament comprising at least one biological molecule or vector, which method comprises the following steps of which one or more are performed in an aseptic enclosure which has been surfaced sterilized using hydrogen peroxide:
(a) formulating the biological molecule or vector with one or more excipients including an antioxidant, to produce a biological medicament comprising an antioxidant;
(b) filling containers with the biological medicament; and
(c) sealing or partially sealing the containers.

2. The method according to claim 1, wherein the hydrogen peroxide used for sterilization is in vaporous form (VHP) or aerosolized form (aHP).

3. The method according to claim 1, wherein the antioxidant is an amino acid or methionine.

4. (canceled)

5. The method according to claim 1, wherein the biological medicament is an immunogenic composition or vaccine and the biological molecule or vector is an antigen or a vector encoding an antigen.

6. The method according to claim 1 comprising the further step of lyophilising (freeze drying) the biological medicament, said lyophilising comprising the following steps:
(a) a freezing step (below the triple point);
(b) a primary drying step; and
(c) a secondary drying step.

7-19. (canceled)

20. The method according to claim 6, further comprising after the freezing step and before the primary drying step, the step of:
(a) an annealing step;
(b) a controlled nucleation step; or
(c) an annealing step and a controlled nucleation step.

21. The method according to claim 1, wherein methionine is present in the biological medicament in an amount between 0.05 and 50 mM, between 0.1 and 20 mM, between 0.1 and 15 mM, between 0.1 and 5 mM, or between 0.5 and 15 mM.

22. The method of claim 1, wherein the biological medicament further comprises an RSV prefusion F antigen.

23. The method of claim 3, wherein the biological medicament comprises:
(a) an H. influenzae protein D antigen; or
(b) an H. influenzae protein D antigen and a PE-PilA fusion protein and a M. catarrhalis UspA2 antigen.

24. The method of claim 23, further comprising:
reconstituting a lyophilised form of the biological medicament in an aqueous solution with an adjuvant.

25. The method of claim 1, wherein the biological medicament comprises an adenovirus vector or ChAd155.

26. The method according to claim 1, wherein the biological medicament is a sterile injectable formulation when in liquid form.

27. An immunogenic composition or vaccine comprising at least one antigen or a vector encoding at least one antigen, formulated with one or more excipients.

28. The immunogenic composition or vaccine of claim 27, wherein the one or more excipients includes an antioxidant, an amino acid, or methionine.

29. The immunogenic composition or vaccine of claim 28, wherein the immunogenic composition or vaccine is in a lyophilised form, suitable for reconstitution in an aqueous solution comprising an adjuvant.

30. The immunogenic composition or vaccine of claim 28, wherein methionine is present in an amount between 0.05 and 50 mM, between 0.1 and 20 mM, between 0.1 and 15 mM, between 0.1 and 5 mM, or between 0.5 and 15 mM.

31. The immunogenic composition or vaccine of claim 30, further comprising an RSV prefusion F antigen.

32. The immunogenic composition or vaccine of claim 28, further comprising:
(a) an H. influenzae protein D antigen; or
(b) an H. influenzae protein D antigen and a PE-PilA fusion protein and a M. catarrhalis UspA2 antigen.

33. The immunogenic composition or vaccine of claim 32, wherein the lyophilized immunogenic composition or vaccine is suitable for reconstitution with an aqueous solution comprising an adjuvant.

34. The immunogenic composition or vaccine of claim 27, further comprising an adenovirus vector or ChAd155.

35. The immunogenic composition or vaccine according to claim 27, wherein the immunogenic composition or vaccine is a sterile injectable formulation when in liquid form.
